SEQUENCE LISTING (101 sequences)

SEQ ID NO: 1 - DNA, 2565 bp, Artificial Sequence
  Synthetic Polynucleotide
atgagagtgcggggcattcagagaaattggccccagtggtggatttggggcatcctgggcttttggatgctgatgatctgcaacgtcgtgggaaatctgtgggtgaccgtgtattatggcgtgcctgtgtggaaagaggccaagaccacactgttttgcgcctctgatgccaaggcctacgagaaagaagtgcacaacgtctgggccacatatgcttgtgtgcccaccgatcccaatcctcaggaaatcgtcctggaaaacgtgaccgagaacttcaacatgtggaagaacgacatggtggaccagatgcacgaggatattatcagcctgtgggacgagtctctgaagccttgtgtgaaactggctcctctgtgcgtgaccctgaactgcaccaacgtgaatagcaccagagtggtcaacatcaccgacaaagaggaaatcaagaactgcagcttcaacatgaccaccgagctgagagacaagaaacagaaggtgtacgccctgttctatagactggacatcgtgcccctgaacgagaatagacacaacagcagcgagtacagactgatcaactgcaataccagcgccattacacaggcctgtcccaaggtgtccttcgatcccatccctatccattattgtgcccctgccggctatgccatcctgaagtgcaacaacaagacctttaatggcaccggcccctgtacaaatgtgtctaccgtgcagtgtacacacggaatcaagcctgtggtgtccacccagctgctgtttaatggctctctggccgaggaagagatcatcatcagaagcgagaacctgaccaacaacgccaagacaatcatcgtgcatctgaatgagagcgtggagatcaattgcaccagacccaacaacaacaccagaaagagcatcagaatcggccctggacagacattctatgccacaggcgagatcatcggagatattagacaggcccactgcaatgtgtccagagccaagtggaatgagacactgcagagagtgggcaagaagctgaaagagcacttccccaacaagaccatcaagttcaatagcagcagcggcggagatctggaaatcaccacccacagcttcaactgcagaggcgagttcttctactgtaacaccagcggcctgtttaatagcacctggtcccagaatgataccggcgtgagcaatagcaccgagagcaacgataccatcatcctgccctgcagaatcaagcagatcatcaatatgtggcaagaggtcggcagagctatgtatgctcctcctatcgccggcaatatcacctgcaagagcaacattacaggcctgctgctcgtcagagatggcggcaacaacaataccaccgagacattcagacctggcggcggaaacatgaaggacaattggcggagcgagctgtacaagtacaaggtggtggagattaaaccactgggcgtggctcctacaagagctaagagaagagtggtggagagggaaaaaagagccgtgggcattggagctgtgtttctgggatttctgggcgctgctggatctacaatgggagccgcctctattactctgacagtgcaggctagactgctgctgtctggaatcgtgcagcagcagaacaatctgctgagggccattgaagcacagcagcatctgctgcagctgacagtgtggggaattaaacagctgcaggccagagtgctggcagtggagagatacctgaaggatcagcagctcctgggaatttggggctgtagcggcaagctgatctgtaccaccaacgtgccttggaactccagctggtccaataagagccaggaagagatttggaacaacatgacctggatggaatgggagagagagatcgacaattacaccggcctgatctacacactgatcgaggaaagccagaaccagcaggaaaagaacgagcaggaactgctggaactggataaatgggccagcctgtggaattggttcgacatcaccaactggctgtggtacatcaagatcttcatcatgatcgtgggcggactgatcggcctgagaatcatctttgccgtgctgtccattgtgaatagagtgcggcagggctattctcctctgagcttccagacaagactgcctgctcctagaggacctgatagacctgagggaatcgaggaagagggcggcgaaagagacagagacagatccatcagactggtgtctggatttctggctctggcctgggatgatctgagaaacctgtgcctgttcagctaccacagactgagagactttatcctgatcgccgccagaacagtggaactgctcggcagatcttctctgagaggactgcagaggggatgggaagctctgaagtacctgggctctctggtgcagtattggggcctggaactgaagaagtctgccatcagcctgctggatacaattgccattgccgtggccgagggcacagatagaatcatcgaggtggtgcagagaatctgcagagccattctgaacatccccagaagaatcagacagggatttgaagccgctctgctgtga

SEQ ID NO: 2 - DNA, 2529 bp, Artificial Sequence
  Synthetic Polynucleotide
atgagagtgaagggcatcagaaagaactaccagcacctgtggagatggggaacaatgctgctgggcatgctgatgatttgttctgccgctgaacagctgtgggtgaccgtgtattatggcgtgcctgtgtggaaagaagccaccaccacactgttttgtgcctctgacgccaaggcctatgatacagaggtgcacaatgtgtgggctacacatgcctgtgtgcctacagatcctaatcctcaggaagtggtcctgggcaacgtgaccgagaacttcaacatgtggaagaacaacatggtggagcagatgcacgaggatattatcagcctgtgggaccagtctctgaagccttgtgtgaagctgacacctctgtgcgtgaccctgaattgcaccgatctgagaaacgccaccaatacaacaagctccagctgggagacaatggaaaagggcgagatcaagaactgcagcttcaacatcaccacctccatcagagacaaggtgcagaaagagtacgccctgttctacaaactggacgtggtgcccatcgacgacaacgacaacaccagctacagactgatcagctgcaataccagcgtgattacccaggcctgtcctaaggtgtccttcgagcccatccctattcattattgcgcccctgccggctttgccatcctgaagtgcaacgacaagaagtttaacggcaccggcccttgcaaaaatgtgtccaccgtgcagtgtacacacggaatcagacctgtggtgtctacacagctgctgctgaatggatctctggctgaggaagaggtggtcatcagaagcgagaactttaccaacaacgccaagaccatcatcgtgcagctgaatgagagcgtcgtgatcaactgcaccagacccaacaacaataccagaaagtccgtgagaattggccctggacaggccttttatgccaccggcgacatcatcggagatattagacaggcccactgcaatatcagccggaccaagtggaacaacaccctgaaccagatcgtgaagaagctgagagagcagttcggcaacaagaccatcgtgttcaatcagtctagcggcggagatcctgagatcgtgatgcacagctttaactgtggcggcgagttcttctactgtaacaccacccagctgttcaatagcacctggaacagcaccgagagaaacgataccatcaccctgccttgcagaatcaagcagattgtgaacatgtggcaagaggtcggcaaagccatgtacgcccctccaatcagaggccagatcagatgcagcagcaatatcacaggcctgctgctgacaagagatggcggcaacaacaacaccaacgagacattcagacctggcgggggagatatgagagacaattggcggagcgagctgtacaagtacaaggtggtcaagatcgaacctctgggagtggctcctacaaaggccaaaagacgggtggtgcagagggaaaaaagagctgtgggcctgggagctatgtttctgggatttctgggcgctgctggatctacaatgggagccgcctctctgacactgacagtgcaggctaggcagctgctgtctggaattgtgcagcagcagagcaatctgctgagagctattgaagcccagcagcacatgctgcagctgacagtgtggggaatcaaacagctgcagaccagagtgctggccatcgagagatacctgaaggatcagcagctcctgggactgtggggatgttctggcaagctgatctgtacaaccgctgtgccttggaatgccagctggtccaacaagagcctgaacgagatctgggacaacatgacctggatgcagtgggacagagagatcagcaactacaccgacaccatctacaggctgctggaagatagccagaaccagcaggaaaagaacgaacaggatctgctggctctggataaatgggctagcctgtggtcttggtttgacatcagcaactggctgtggtacatccggatcttcatcatgatcgtgggcggactgattggcctgagaatcgtgtttgccgtgctgtccattgtgaatagagtgcggaagggctactctcctctgagctttcagaccctgacacctaatcctagaggccctgacagactgggcagaatcgaagaagaaggcggcgagcaggatagagatagaagcatccggctggtcaatggatttctggccctggcttgggatgatctgagaagcctgtgcctgtttagctaccacagactgagagatctgctgctgatcgtgacaagaatcgtggaactgctgggaagaagaggctgggaagccctgaagtattggtggaacctgctgcagtattggagccaggaactgaagaattctgccgtgagcctgctgaatgctacagccattgctgtggccgaaggcacagatagagtgattgaggtggtgcagcgggcttatagagccatcctgcacatccccagaagaatcagacagggactggaaagggctctgctgtga

SEQ ID NO: 3 - DNA, 1500 bp, Artificial Sequence
  Synthetic Polynucleotide
atgggagccagagcttctattctgagaggcggcaagctggataagtgggagaagatcagactgaggcctggcggcaagaaacactacatgctgaagcacattgtgtgggccagcagagaactggaaagattcgccgtgaatcctggcctgctggaaacatctgagggctgtagacagattctgggacagctgcagccttctctgcagacaggcagcgaggaactgaagtccctgtacaataccgtggccacactgtattgtgtgcaccagagaatcgacgtgaaggataccaaagaggccctggaaaagatcgaggaagaacagaacaagagcaagaagaaagcacagcaggctgccgctgatacaggaaatagcagccaggtctcccagaattaccccatcgtgcagaatctgcagggacagatggtgcatcaggccatcagccctagaacactgaatgcctgggtgaaagtggtggaggaaaaggcctttagccccgaagtgatccctatgtttagcgctctgtctgaaggtgctaccccccaggatctgaacatgatgctgaatatcgtgggaggacatcaggctgctatgcagatgctgaaagagacaatcaacgaagaggccgccgaatgggatagagtgcatcctgtgcatgctggacctattccacctggacagatgagagagcccagaggatctgatattgccggcagcacatctacactgcaagaacagatcggctggatgaccaacaatcctcctatccctgtgggcgagatctacaagagatggatcatcctgggcctgaataagatcgtgcggatgtacagccctaccagcatcctggatattagacagggccccaaagagcctttcagagactacgtggacagattctacaagacactgagagccgaacaggccacacaagaggtgaagaactggatgaccgaaaccctgctggtgcagaatgccaatcccgattgcaagacaatcctgaaagctctgggacctgctgctacactggaagagatgatgacagcttgtcagggtgtcggaggaccaggacagaaagccagactgatggccgaagctctgaaagaagctctggcccctgtccctattccttttgctgctgcccagcagagaggacctagaaagcccatcaagtgctggaactgtggcaaagaaggacatagcgccagacagtgtagagcacctagaaggcagggctgttggaaatgtggaaaagagggccaccagatgaaggactgtaacgagagacaggccaactttctgggcaagatctggccttctaataagggcagacccggcaattttctgcagagcagacctgaacctacagcccctcccgaggaaagctttagattcggcgaggaaaccaccacaccttctcagaagcaggaacccatcgacaaagaactgtaccctctggccagcctgaagtctctgttcggcaatgatcccctgagccag

SEQ ID NO: 4 - DNA, 1500 bp, Artificial Sequence
  Synthetic Polynucleotide
atgggagctagagcatctgtgctgtctggcggaaaactggatgcctgggagaagattagactgaggcctggcggcaagaagaagtacagactgaagcatctcgtgtgggctagcagagaactggaaagattcgccctgaatcctggcctgctggaaacaagcgagggctgcaagcagatcattaaacagctgcagccagctctgcagacaggaaccgaggaactgagaagcctgtttaataccgtggccaccctgtattgtgtgcaccagcggatcgaagtgaaggataccaaagaggccctggacaagatcgaggaagaacagaacaagagccagcagaaaacacagcaggccaaagccgctgatggcaaggtgtcccagaattaccctatcgtgcagaatgctcagggacagatggtgcatcaggccctgtctccaagaacactgaacgcctgggtgaaagtgatcgaggaaaaggccttctctcccgaagtgatccctatgtttaccgctctgtctgaaggtgctacccctcaggatctgaacaccatgctgaatacagtgggaggacatcaggctgctatgcagatgctgaaggacaccattaatgaagaggccgccgaatgggatagactgcatcctgtgcatgctggacctattgctccaggacagatgagagagcccagaggatctgatattgccggcaccacatctacactgcaagaacagatcgcctggatgaccagcaatcctcctatccctgtgggcgacatctacaagagatggatcatcctgggcctggataagatcgtgcggatgtacagccctgtgtccatcctggatattaagcagggccccaaagagcctttcagagactacgtggacagattcttcaagacactgagagccgaacaggccacacaggacgtgaagaactggatgaccgataccctgctggtccagaatgccaatcccgattgcaagacaattctgagagcactgggacctggtgctacactggaagagatgatgacagcttgtcagggtgtcggaggaccatctcagaaagccagactgatggccgaagctctgaaagaagctctggcccctgtccctattccttttgctgctgcccagcagagaggacctagaaagcccatcaagtgctggaactgtggcaaagaaggacatagcgccagacagtgtagagcacctagaaggcagggctgttggaaatgtggaagagaaggccaccagatgaaggattgtaccgagagacaggccaactttctgggcaagatctggccttcacacaagggcagacctggcaactttctgcagaacagacctgaacctacagctcctcctgctgaaccaacagcaccacctgccgagagcttcagatttgaggaaaccacacccgctcctaagcaggaacctaaggacagagagcctctgacaagcctgaagtccctgtttggctctgatcctctgagccag

SEQ ID NO: 5 - DNA, 1515 bp, Artificial Sequence
  Synthetic Polynucleotide
atgggagctagagcatctgtgctgtctggcggagaactggatagatgggagaagatcagactgaggcctggcggcaagaagaagtacaagctgaagcacattgtgtgggctagcagagagctggacagatttgccctgaatcctggactgctggaaacagctgagggctgtcagcagatcattgaacagctgcagcctgccctgaaaacaggcaccgaggaactgaagtccctgtttaataccgtggccaccctgtactgtgtgcacgagaagatcgaagtgcgggatacaaaagaggccctggacaagatcgaggaaatccagaacaagagcaagcagaaaacacagcaggctgccgctgatacaggatctagcagcaaggtgtcccagaattaccccatcgtgcagaatattcagggacagatggtgcaccagcctatcagccctagaacactgaatgcctgggtgaaagtggtggaggaaaagggcttcaaccccgaagtgatccctatgttttctgctctggccgaaggtgctacacctcaggacctgaacaccatgctgaatacaattggaggacatcaggccgccatgcagatcctgaaggacaccattaatgaggaagccgccgattgggatagactgcatcctgtgcatgctggacctgtggctccaggacagatgagagagcccagaggatctgatattgccggcaccacaagcaatctgcaagaacagatcggctggatgacctctaatcctcctgtgcctgtgggcgagatctataagagatggatcgtgctgggcctgaataagatcgtgcggatgtacagccctgtgtccatcctggatattagacagggccccaaagagtccttcagagactacgtggacagattctacaagacactgagagccgaacaggccagccaggatgtgaagaactggatgaccgagacactgctgatccagaacgccaatcccgattgcaagtctattctgagagcactgggacctggtgctagcctggaagagatgatgacagcttgtcagggtgtcggaggaccatctcagaaagccagactgatggccgaagctctgaaagaagctctggcccctgtccctattccttttgctgctgcccagcagagaggacctagaaagcccatcaagtgctggaactgtggcaaagaaggacatagcgccagacagtgtagagcacctagaaggcagggctgttggaaatgtggacaggaaggccaccagatgaaggattgtagcgagagacaggccaactttctgggcaagatctggccttctagcaagggcagacccggcaattttcctcagagcagacctgaacctacagctcctctggaaccaacagctccacctgccgagagctttggctttggcgaggaaatcacacctagccagaaacaggaacagaaggacaaagagctgtatcctctggccagcctgagaagcctgtttggcaatgaccctagcagccag

SEQ ID NO: 6 - DNA, 2577 bp, Artificial Sequence
  Synthetic Polynucleotide
atgagagtgaaagaaacccagatgaactggcccaatctgtggaagtggggaacactgatcctgggcctggtcatcatttgtagcgccagcgataatctgtgggtgacagtgtattatggcgtgcctgtgtggagagatgccgagacaacactgttttgcgcctctgatgccaaggcctatgagagagaggtgcacaatatttgggccacacacgcctgtgtgcctacagatcctagccctcaggaaatccacctggaaaacgtgaccgaggaattcaacatgtggaagaacgacatggtggagcagatgcacaccgatattatcagcctgtgggaccagtctctgaaaccttgtgtgcagctgacacctctgtgtgtgaccctgaactgcagcaacgtgaacaacaccagaaacagcaccaacaccgtgaacaataccatgaacggcgagatgaagaactgcagcttcaacatcaccaccgagatcagagataagaagcagaaggcctacgccctgttttacaagctggacatcgtgcctctgaagggcagcaatagcagcgagtacatcctgatcaactgcaacaccagcacaattacccaggcctgccccaaagtgacattcgagcccatccctatccattattgtacacctgccggctacgccatcctgaagtgcaacgacaagacctttaatggcaccggcccctgcaataatgtgtctaccgtgcagtgtacacacggcatcaagcctgtgattagcacccagctgctgctgaatggatctctggccgagggcgagatcatcatcagaagcgagaacctgaccgacaatgccaagacaatcatcgtgcacctgaacaagagcgtggagatcgtgtgtaccagacccggcaacaataccagaaagtccatccacattggccctggcagagccttttatgccaccggcgacatcatcggaaatatcagacaggcccactgcaatctgagcagaaccgactggaataacaccctgaagcagatcgccgagaagctgaaagagcagttcaacaagaccatcatcttcaatcagagcagcggcggagatcctgagatcaccacccacagctttaattgtggcggcgagttcttctactgcaataccaccaagctgttcaacagcacctggaatgatacaggcagcatgcccgagagcaacaacaccaacggcaacatcaccctgcagtgcagaatcaagcagatcatcaatatgtggcagcgagtgggacaggctatgtatgcccctcctatcgagggcaatatcacctgtagaagcaacatcacaggcctgatcctgacaagagatggcggcaatcacagcagaagcgacaacaacaccgagatctttagacctggcggcggaaacatgagagacaactggcggaacgagctgtacaagtacaaggtggtgcagatcgaacctctgggaatcgctcctaccaaggccaaaagacgggtggtggagagggaaaaaagagctgtgggactgggagctgtgtttctgggatttctgggcacagctggatctacaatgggagccgcctctatgacactgacagtgcaggctagacaggtgctgtctggaattgtgcagcagcagagcaatctgctgaaagccattgaagcacagcagcacctgctgaaactgacagtgtggggcattaaacagctgcaggccaggattctggctgtggagcgctatctgagagatcagcagctcctgggcatttggggctactctggcaagctgatctgtaccaccaccgtgccttggaataccagctggtccaacaagagcctgaaccagatctgggacaatatgacctggctgcagtgggacaaagagatctccaactacaccaacaccatctacagactgctggaagagagccagaaccagcaggaaaagaacgagaaagacctgctggccctggattcttggaagaacctgtggaattggttcgacatcaccaagtggctgtggtacatcaagatcttcatcatgatcgtgggaggactcgtgggactgagaatcgtgttcaccgtgctgtccatcattaatagagtgcggcagggctattctcctctgtctctgcagacactgacacaccaccagagagagcctgatagacccgagagaatcgaagaaggtggcggcgaacaggatagagatagatccgtgagactggtgtctggatttctggccctgatctgggatgatctgagaagcctgtgcctgtttagctatcaccagctgcgggactttattctgatcgtggccagaacagtggaactgctgggccactcttctctgaaaggactgagactgggatgggagggactgaagtatctgtggaacctgctgcagtactggattcaggaactgaagaacagcgccatcagcctgctgaatacaacagccattgtggtggccgaaggcacagatagagtgatcgaagtgctgcagagagctggcagagctatcctgcacatccccacaagaatcagacagggctttgaaagggctctgctgtga

SEQ ID NO: 7 - DNA, 5930 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgggagctagagcatctgtgctgtctggcggagaactggatagatgggagaagatcagactgaggcctggcggcaagaagaagtacaagctgaagcacattgtgtgggctagcagagagctggacagatttgccctgaatcctggactgctggaaacagctgagggctgtcagcagatcattgaacagctgcagcctgccctgaaaacaggcaccgaggaactgaagtccctgtttaataccgtggccaccctgtactgtgtgcacgagaagatcgaagtgcgggatacaaaagaggccctggacaagatcgaggaaatccagaacaagagcaagcagaaaacacagcaggctgccgctgatacaggatctagcagcaaggtgtcccagaattaccccatcgtgcagaatattcagggacagatggtgcaccagcctatcagccctagaacactgaatgcctgggtgaaagtggtggaggaaaagggcttcaaccccgaagtgatccctatgttttctgctctggccgaaggtgctacacctcaggacctgaacaccatgctgaatacaattggaggacatcaggccgccatgcagatcctgaaggacaccattaatgaggaagccgccgattgggatagactgcatcctgtgcatgctggacctgtggctccaggacagatgagagagcccagaggatctgatattgccggcaccacaagcaatctgcaagaacagatcggctggatgacctctaatcctcctgtgcctgtgggcgagatctataagagatggatcgtgctgggcctgaataagatcgtgcggatgtacagccctgtgtccatcctggatattagacagggccccaaagagtccttcagagactacgtggacagattctacaagacactgagagccgaacaggccagccaggatgtgaagaactggatgaccgagacactgctgatccagaacgccaatcccgattgcaagtctattctgagagcactgggacctggtgctagcctggaagagatgatgacagcttgtcagggtgtcggaggaccatctcagaaagccagactgatggccgaagctctgaaagaagctctggcccctgtccctattccttttgctgctgcccagcagagaggacctagaaagcccatcaagtgctggaactgtggcaaagaaggacatagcgccagacagtgtagagcacctagaaggcagggctgttggaaatgtggacaggaaggccaccagatgaaggattgtagcgagagacaggccaactttctgggcaagatctggccttctagcaagggcagacccggcaattttcctcagagcagacctgaacctacagctcctctggaaccaacagctccacctgccgagagctttggctttggcgaggaaatcacacctagccagaaacaggaacagaaggacaaagagctgtatcctctggccagcctgagaagcctgtttggcaatgaccctagcagccagtgatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 5915 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgggagccagagcttctattctgagaggcggcaagctggataagtgggagaagatcagactgaggcctggcggcaagaaacactacatgctgaagcacattgtgtgggccagcagagaactggaaagattcgccgtgaatcctggcctgctggaaacatctgagggctgtagacagattctgggacagctgcagccttctctgcagacaggcagcgaggaactgaagtccctgtacaataccgtggccacactgtattgtgtgcaccagagaatcgacgtgaaggataccaaagaggccctggaaaagatcgaggaagaacagaacaagagcaagaagaaagcacagcaggctgccgctgatacaggaaatagcagccaggtctcccagaattaccccatcgtgcagaatctgcagggacagatggtgcatcaggccatcagccctagaacactgaatgcctgggtgaaagtggtggaggaaaaggcctttagccccgaagtgatccctatgtttagcgctctgtctgaaggtgctaccccccaggatctgaacatgatgctgaatatcgtgggaggacatcaggctgctatgcagatgctgaaagagacaatcaacgaagaggccgccgaatgggatagagtgcatcctgtgcatgctggacctattccacctggacagatgagagagcccagaggatctgatattgccggcagcacatctacactgcaagaacagatcggctggatgaccaacaatcctcctatccctgtgggcgagatctacaagagatggatcatcctgggcctgaataagatcgtgcggatgtacagccctaccagcatcctggatattagacagggccccaaagagcctttcagagactacgtggacagattctacaagacactgagagccgaacaggccacacaagaggtgaagaactggatgaccgaaaccctgctggtgcagaatgccaatcccgattgcaagacaatcctgaaagctctgggacctgctgctacactggaagagatgatgacagcttgtcagggtgtcggaggaccaggacagaaagccagactgatggccgaagctctgaaagaagctctggcccctgtccctattccttttgctgctgcccagcagagaggacctagaaagcccatcaagtgctggaactgtggcaaagaaggacatagcgccagacagtgtagagcacctagaaggcagggctgttggaaatgtggaaaagagggccaccagatgaaggactgtaacgagagacaggccaactttctgggcaagatctggccttctaataagggcagacccggcaattttctgcagagcagacctgaacctacagcccctcccgaggaaagctttagattcggcgaggaaaccaccacaccttctcagaagcaggaacccatcgacaaagaactgtaccctctggccagcctgaagtctctgttcggcaatgatcccctgagccagtgatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 5915 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgggagctagagcatctgtgctgtctggcggaaaactggatgcctgggagaagattagactgaggcctggcggcaagaagaagtacagactgaagcatctcgtgtgggctagcagagaactggaaagattcgccctgaatcctggcctgctggaaacaagcgagggctgcaagcagatcattaaacagctgcagccagctctgcagacaggaaccgaggaactgagaagcctgtttaataccgtggccaccctgtattgtgtgcaccagcggatcgaagtgaaggataccaaagaggccctggacaagatcgaggaagaacagaacaagagccagcagaaaacacagcaggccaaagccgctgatggcaaggtgtcccagaattaccctatcgtgcagaatgctcagggacagatggtgcatcaggccctgtctccaagaacactgaacgcctgggtgaaagtgatcgaggaaaaggccttctctcccgaagtgatccctatgtttaccgctctgtctgaaggtgctacccctcaggatctgaacaccatgctgaatacagtgggaggacatcaggctgctatgcagatgctgaaggacaccattaatgaagaggccgccgaatgggatagactgcatcctgtgcatgctggacctattgctccaggacagatgagagagcccagaggatctgatattgccggcaccacatctacactgcaagaacagatcgcctggatgaccagcaatcctcctatccctgtgggcgacatctacaagagatggatcatcctgggcctggataagatcgtgcggatgtacagccctgtgtccatcctggatattaagcagggccccaaagagcctttcagagactacgtggacagattcttcaagacactgagagccgaacaggccacacaggacgtgaagaactggatgaccgataccctgctggtccagaatgccaatcccgattgcaagacaattctgagagcactgggacctggtgctacactggaagagatgatgacagcttgtcagggtgtcggaggaccatctcagaaagccagactgatggccgaagctctgaaagaagctctggcccctgtccctattccttttgctgctgcccagcagagaggacctagaaagcccatcaagtgctggaactgtggcaaagaaggacatagcgccagacagtgtagagcacctagaaggcagggctgttggaaatgtggaagagaaggccaccagatgaaggattgtaccgagagacaggccaactttctgggcaagatctggccttcacacaagggcagacctggcaactttctgcagaacagacctgaacctacagctcctcctgctgaaccaacagcaccacctgccgagagcttcagatttgaggaaaccacacccgctcctaagcaggaacctaaggacagagagcctctgacaagcctgaagtccctgtttggctctgatcctctgagccagtgatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 6996 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgagagtgaaagaaacccagatgaactggcccaatctgtggaagtggggaacactgatcctgggcctggtcatcatttgtagcgccagcgataatctgtgggtgacagtgtattatggcgtgcctgtgtggagagatgccgagacaacactgttttgcgcctctgatgccaaggcctatgagagagaggtgcacaatatttgggccacacacgcctgtgtgcctacagatcctagccctcaggaaatccacctggaaaacgtgaccgaggaattcaacatgtggaagaacgacatggtggagcagatgcacaccgatattatcagcctgtgggaccagtctctgaaaccttgtgtgcagctgacacctctgtgtgtgaccctgaactgcagcaacgtgaacaacaccagaaacagcaccaacaccgtgaacaataccatgaacggcgagatgaagaactgcagcttcaacatcaccaccgagatcagagataagaagcagaaggcctacgccctgttttacaagctggacatcgtgcctctgaagggcagcaatagcagcgagtacatcctgatcaactgcaacaccagcacaattacccaggcctgccccaaagtgacattcgagcccatccctatccattattgtacacctgccggctacgccatcctgaagtgcaacgacaagacctttaatggcaccggcccctgcaataatgtgtctaccgtgcagtgtacacacggcatcaagcctgtgattagcacccagctgctgctgaatggatctctggccgagggcgagatcatcatcagaagcgagaacctgaccgacaatgccaagacaatcatcgtgcacctgaacaagagcgtggagatcgtgtgtaccagacccggcaacaataccagaaagtccatccacattggccctggcagagccttttatgccaccggcgacatcatcggaaatatcagacaggcccactgcaatctgagcagaaccgactggaataacaccctgaagcagatcgccgagaagctgaaagagcagttcaacaagaccatcatcttcaatcagagcagcggcggagatcctgagatcaccacccacagctttaattgtggcggcgagttcttctactgcaataccaccaagctgttcaacagcacctggaatgatacaggcagcatgcccgagagcaacaacaccaacggcaacatcaccctgcagtgcagaatcaagcagatcatcaatatgtggcagcgagtgggacaggctatgtatgcccctcctatcgagggcaatatcacctgtagaagcaacatcacaggcctgatcctgacaagagatggcggcaatcacagcagaagcgacaacaacaccgagatctttagacctggcggcggaaacatgagagacaactggcggaacgagctgtacaagtacaaggtggtgcagatcgaacctctgggaatcgctcctaccaaggccaaaagacgggtggtggagagggaaaaaagagctgtgggactgggagctgtgtttctgggatttctgggcacagctggatctacaatgggagccgcctctatgacactgacagtgcaggctagacaggtgctgtctggaattgtgcagcagcagagcaatctgctgaaagccattgaagcacagcagcacctgctgaaactgacagtgtggggcattaaacagctgcaggccaggattctggctgtggagcgctatctgagagatcagcagctcctgggcatttggggctactctggcaagctgatctgtaccaccaccgtgccttggaataccagctggtccaacaagagcctgaaccagatctgggacaatatgacctggctgcagtgggacaaagagatctccaactacaccaacaccatctacagactgctggaagagagccagaaccagcaggaaaagaacgagaaagacctgctggccctggattcttggaagaacctgtggaattggttcgacatcaccaagtggctgtggtacatcaagatcttcatcatgatcgtgggaggactcgtgggactgagaatcgtgttcaccgtgctgtccatcattaatagagtgcggcagggctattctcctctgtctctgcagacactgacacaccaccagagagagcctgatagacccgagagaatcgaagaaggtggcggcgaacaggatagagatagatccgtgagactggtgtctggatttctggccctgatctgggatgatctgagaagcctgtgcctgtttagctatcaccagctgcgggactttattctgatcgtggccagaacagtggaactgctgggccactcttctctgaaaggactgagactgggatgggagggactgaagtatctgtggaacctgctgcagtactggattcaggaactgaagaacagcgccatcagcctgctgaatacaacagccattgtggtggccgaaggcacagatagagtgatcgaagtgctgcagagagctggcagagctatcctgcacatccccacaagaatcagacagggctttgaaagggctctgctgtgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 6984 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgagagtgcggggcattcagagaaattggccccagtggtggatttggggcatcctgggcttttggatgctgatgatctgcaacgtcgtgggaaatctgtgggtgaccgtgtattatggcgtgcctgtgtggaaagaggccaagaccacactgttttgcgcctctgatgccaaggcctacgagaaagaagtgcacaacgtctgggccacatatgcttgtgtgcccaccgatcccaatcctcaggaaatcgtcctggaaaacgtgaccgagaacttcaacatgtggaagaacgacatggtggaccagatgcacgaggatattatcagcctgtgggacgagtctctgaagccttgtgtgaaactggctcctctgtgcgtgaccctgaactgcaccaacgtgaatagcaccagagtggtcaacatcaccgacaaagaggaaatcaagaactgcagcttcaacatgaccaccgagctgagagacaagaaacagaaggtgtacgccctgttctatagactggacatcgtgcccctgaacgagaatagacacaacagcagcgagtacagactgatcaactgcaataccagcgccattacacaggcctgtcccaaggtgtccttcgatcccatccctatccattattgtgcccctgccggctatgccatcctgaagtgcaacaacaagacctttaatggcaccggcccctgtacaaatgtgtctaccgtgcagtgtacacacggaatcaagcctgtggtgtccacccagctgctgtttaatggctctctggccgaggaagagatcatcatcagaagcgagaacctgaccaacaacgccaagacaatcatcgtgcatctgaatgagagcgtggagatcaattgcaccagacccaacaacaacaccagaaagagcatcagaatcggccctggacagacattctatgccacaggcgagatcatcggagatattagacaggcccactgcaatgtgtccagagccaagtggaatgagacactgcagagagtgggcaagaagctgaaagagcacttccccaacaagaccatcaagttcaatagcagcagcggcggagatctggaaatcaccacccacagcttcaactgcagaggcgagttcttctactgtaacaccagcggcctgtttaatagcacctggtcccagaatgataccggcgtgagcaatagcaccgagagcaacgataccatcatcctgccctgcagaatcaagcagatcatcaatatgtggcaagaggtcggcagagctatgtatgctcctcctatcgccggcaatatcacctgcaagagcaacattacaggcctgctgctcgtcagagatggcggcaacaacaataccaccgagacattcagacctggcggcggaaacatgaaggacaattggcggagcgagctgtacaagtacaaggtggtggagattaaaccactgggcgtggctcctacaagagctaagagaagagtggtggagagggaaaaaagagccgtgggcattggagctgtgtttctgggatttctgggcgctgctggatctacaatgggagccgcctctattactctgacagtgcaggctagactgctgctgtctggaatcgtgcagcagcagaacaatctgctgagggccattgaagcacagcagcatctgctgcagctgacagtgtggggaattaaacagctgcaggccagagtgctggcagtggagagatacctgaaggatcagcagctcctgggaatttggggctgtagcggcaagctgatctgtaccaccaacgtgccttggaactccagctggtccaataagagccaggaagagatttggaacaacatgacctggatggaatgggagagagagatcgacaattacaccggcctgatctacacactgatcgaggaaagccagaaccagcaggaaaagaacgagcaggaactgctggaactggataaatgggccagcctgtggaattggttcgacatcaccaactggctgtggtacatcaagatcttcatcatgatcgtgggcggactgatcggcctgagaatcatctttgccgtgctgtccattgtgaatagagtgcggcagggctattctcctctgagcttccagacaagactgcctgctcctagaggacctgatagacctgagggaatcgaggaagagggcggcgaaagagacagagacagatccatcagactggtgtctggatttctggctctggcctgggatgatctgagaaacctgtgcctgttcagctaccacagactgagagactttatcctgatcgccgccagaacagtggaactgctcggcagatcttctctgagaggactgcagaggggatgggaagctctgaagtacctgggctctctggtgcagtattggggcctggaactgaagaagtctgccatcagcctgctggatacaattgccattgccgtggccgagggcacagatagaatcatcgaggtggtgcagagaatctgcagagccattctgaacatccccagaagaatcagacagggatttgaagccgctctgctgtgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 6948 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgagagtgaagggcatcagaaagaactaccagcacctgtggagatggggaacaatgctgctgggcatgctgatgatttgttctgccgctgaacagctgtgggtgaccgtgtattatggcgtgcctgtgtggaaagaagccaccaccacactgttttgtgcctctgacgccaaggcctatgatacagaggtgcacaatgtgtgggctacacatgcctgtgtgcctacagatcctaatcctcaggaagtggtcctgggcaacgtgaccgagaacttcaacatgtggaagaacaacatggtggagcagatgcacgaggatattatcagcctgtgggaccagtctctgaagccttgtgtgaagctgacacctctgtgcgtgaccctgaattgcaccgatctgagaaacgccaccaatacaacaagctccagctgggagacaatggaaaagggcgagatcaagaactgcagcttcaacatcaccacctccatcagagacaaggtgcagaaagagtacgccctgttctacaaactggacgtggtgcccatcgacgacaacgacaacaccagctacagactgatcagctgcaataccagcgtgattacccaggcctgtcctaaggtgtccttcgagcccatccctattcattattgcgcccctgccggctttgccatcctgaagtgcaacgacaagaagtttaacggcaccggcccttgcaaaaatgtgtccaccgtgcagtgtacacacggaatcagacctgtggtgtctacacagctgctgctgaatggatctctggctgaggaagaggtggtcatcagaagcgagaactttaccaacaacgccaagaccatcatcgtgcagctgaatgagagcgtcgtgatcaactgcaccagacccaacaacaataccagaaagtccgtgagaattggccctggacaggccttttatgccaccggcgacatcatcggagatattagacaggcccactgcaatatcagccggaccaagtggaacaacaccctgaaccagatcgtgaagaagctgagagagcagttcggcaacaagaccatcgtgttcaatcagtctagcggcggagatcctgagatcgtgatgcacagctttaactgtggcggcgagttcttctactgtaacaccacccagctgttcaatagcacctggaacagcaccgagagaaacgataccatcaccctgccttgcagaatcaagcagattgtgaacatgtggcaagaggtcggcaaagccatgtacgcccctccaatcagaggccagatcagatgcagcagcaatatcacaggcctgctgctgacaagagatggcggcaacaacaacaccaacgagacattcagacctggcgggggagatatgagagacaattggcggagcgagctgtacaagtacaaggtggtcaagatcgaacctctgggagtggctcctacaaaggccaaaagacgggtggtgcagagggaaaaaagagctgtgggcctgggagctatgtttctgggatttctgggcgctgctggatctacaatgggagccgcctctctgacactgacagtgcaggctaggcagctgctgtctggaattgtgcagcagcagagcaatctgctgagagctattgaagcccagcagcacatgctgcagctgacagtgtggggaatcaaacagctgcagaccagagtgctggccatcgagagatacctgaaggatcagcagctcctgggactgtggggatgttctggcaagctgatctgtacaaccgctgtgccttggaatgccagctggtccaacaagagcctgaacgagatctgggacaacatgacctggatgcagtgggacagagagatcagcaactacaccgacaccatctacaggctgctggaagatagccagaaccagcaggaaaagaacgaacaggatctgctggctctggataaatgggctagcctgtggtcttggtttgacatcagcaactggctgtggtacatccggatcttcatcatgatcgtgggcggactgattggcctgagaatcgtgtttgccgtgctgtccattgtgaatagagtgcggaagggctactctcctctgagctttcagaccctgacacctaatcctagaggccctgacagactgggcagaatcgaagaagaaggcggcgagcaggatagagatagaagcatccggctggtcaatggatttctggccctggcttgggatgatctgagaagcctgtgcctgtttagctaccacagactgagagatctgctgctgatcgtgacaagaatcgtggaactgctgggaagaagaggctgggaagccctgaagtattggtggaacctgctgcagtattggagccaggaactgaagaattctgccgtgagcctgctgaatgctacagccattgctgtggccgaaggcacagatagagtgattgaggtggtgcagcgggcttatagagccatcctgcacatccccagaagaatcagacagggactggaaagggctctgctgtgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 1509 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatag

SEQ ID NO: 14 - DNA, 1500 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaa

SEQ ID NO: 15 - DNA, 1497 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccacagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 16 - DNA, 1522 bp, Artificial Sequence
  Synthetic Polynucleotide
gtcgacgccaccatgggcgccagggccagcgtgctgtctggcggcgagctggacagatgggagaagatccggctgcggcctggcggcaagaagaagtaccggctgaagcacatcgtgtgggccagccgggagctggaacggttcgccgtgaaccccggcctgctggaaaccagcgagggctgccggcagatcctgggccagctgcagcccagcctgcagaccggcagcgaggaactgcggagcctgtacaacaccgtggccaccctgtactgcgtgcaccagcggatcgagatcaaggacaccaaagaggccctggaaaagatcgaggaagagcagaacaagtccaagaagaaggcccagcaggctgccgccgacaccggcaacagcagccaggtgtcccagaactaccccatcgtgcagaacatccagggccagatggtgcaccaggccatcagcccccggaccctgaacgcctgggtgaaggtggtggaggaaaaggccttcagccccgaggtgatccccatgttcagcgccctgagcgagggcgccacaccccaggacctgaacaccatgctgaacaccgtgggcggccaccaggccgccatgcagatgctgaaagagaccatcaacgaggaagccgccgagtgggacagagtgcaccccgtgcacgccggacctatcgcccctggccagatgcgggagcccaggggcagcgacatcgccggcacaaccagcacactgcaggaacagatcggctggatgaccaacaacccccccatccccgtgggcgagatctacaagcggtggatcatcctgggcctgaacaagatcgtgcggatgtacagccccgtgagcatcctggacatccggcagggccccaaagagcccttccgggactacgtggaccggttctacaagaccctgcgggccgagcaggccagccaggacgtgaagaactggatgaccgagaccctgctggtgcagaacgccaaccccgactgcaagaccatcctgaaggccctgggccctgccgccaccctggaagagatgatgaccgcctgccagggcgtgggcggacctggccacaaggcccgggtgctggccgaggccatgagccaggtgaccaacagcgccaccatcatgatgcagcggggcaacttccggaaccagagaaagaccgtgaagtgcttcaactgcggcaaagagggccacatcgccaagaactgcagggcccccaggaagaagggctgctggaagtgtggcaaggaagggcaccagatgaaggactgcaccgagcggcaggccaacttcctgggcaagatttggcccagcaacaagggcaggcccggcaacttcctgcagaaccggcccgagcccaccgcccctcccgaggaaagcttccggttcggcgaggaaaccaccacccccagccagaagcaggaacccatcgacaaagagatgtaccccctggcctccctgaagagcctgttcggcaacgaccccagctcccagtaatgaattc

SEQ ID NO: 17 - DNA, 1510 bp, Artificial Sequence
  Synthetic Polynucleotide
gtcgacgccaccatgggcgctagggccagcatcctgaggggcggcaagctggacaagtgggagaagatccggctgcggcctggcggcaagaaacactacatgctgaagcacctggtctgggccagccgggagctggaacggttcgccctgaaccccggcctgctggaaaccagcgagggctgcaagcagatcatcaagcagctgcagcccgccctgcagaccggcaccgaggaactgcggagcctgttcaacaccgtggccaccctgtactgcgtgcacgccgagatcgaagtgcgggacaccaaagaggccctggacaagatcgaggaagagcagaacaagagccagcagaaaacccagcaggccaaagaagccgacggcaaggtctcccagaactaccccatcgtgcagaacctgcagggccagatggtgcaccagcccatcagcccccggaccctgaacgcctgggtgaaggtgatcgaggaaaaggccttcagccccgaggtgatccccatgttcaccgccctgagcgagggcgccacaccccaggacctgaacaccatgctgaacaccgtgggcggccaccaggccgccatgcagatgctgaaggacaccatcaacgaggaagccgccgagtgggaccggctgcaccctgtgcacgccggacctgtggcccctggccagatgcgggagcccaggggcagcgacatcgccggcacaaccagcaacctgcaggaacagatcgcctggatgaccagcaacccccccatccccgtgggcgacatctacaagcggtggatcatcctgggcctgaacaagatcgtgcggatgtacagccccacctccatcctggacatcaagcagggccccaaagagcccttccgggactacgtggaccggttcttcaagaccctgcgggccgagcaggccacccaggacgtgaagaactggatgaccgacaccctgctggtgcagaacgccaaccccgactgcaagaccatcctgcgggccctgggccctggagccaccctggaagagatgatgaccgcctgccagggcgtgggcggacccagccacaaggcccgggtgctggccgaggccatgagccagaccaacagcaccatcctgatgcagcggagcaacttcaagggcagcaagcggatcgtgaagtgcttcaactgcggcaaagagggccacatcgcccggaactgcagggcccccaggaagaagggctgctggaagtgtggcaaggaagggcaccagatgaaggactgcaccgagcggcaggccaacttcctgggcaagatctggccctcccacaagggcaggcccggcaacttcctgcagagcaggcccgagcccacagcccctcccgccgagagcttccggttcgaggaaaccacccctgcccccaagcaggaacccaaggaccgggagcccctgaccagcctgagaagcctgttcggcagcgaccccctgagccagtaatgattcacgtaagggcgaattc

SEQ ID NO: 18 - DNA, 1522 bp, Artificial Sequence
  Synthetic Polynucleotide
gtcgacgccaccatgggcgccagggccagcgtgctgtctggcggcgagctggacagatgggagaagatccggctgcggcctggcggcaagaagaagtaccggctgaagcacatcgtgtgggccagccgggagctggaacggttcgccgtgaaccccggcctgctggaaaccagcgagggctgccggcagatcctgggccagctgcagcccagcctgcagaccggcagcgaggaactgcggagcctgtacaacaccgtggccaccctgtactgcgtgcaccagcggatcgagatcaaggacaccaaagaggccctggaaaagatcgaggaagagcagaacaagtccaagaagaaggcccagcaggctgccgccgacaccggcaacagcagccaggtgtcccagaactaccccatcgtgcagaacatccagggccagatggtgcaccaggccatcagcccccggaccctgaacgcctgggtgaaggtggtggaggaaaaggccttcagccccgaggtgatccccatgttcagcgccctgagcgagggcgccacaccccaggacctgaacaccatgctgaacaccgtgggcggccaccaggccgccatgcagatgctgaaagagaccatcaacgaggaagccgccgagtgggacagagtgcaccccgtgcacgccggacctatcgcccctggccagatgcgggagcccaggggcagcgacatcgccggcacaaccagcacactgcaggaacagatcggctggatgaccaacaacccccccatccccgtgggcgagatctacaagcggtggatcatcctgggcctgaacaagatcgtgcggatgtacagccccgtgagcatcctggacatccggcagggccccaaagagcccttccgggactacgtggaccggttctacaagaccctgcgggccgagcaggccagccaggacgtgaagaactggatgaccgagaccctgctggtgcagaacgccaaccccgactgcaagaccatcctgaaggccctgggccctgccgccaccctggaagagatgatgaccgcctgccagggcgtgggcggacctggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgtggcaaggaagggcaccagatgaaggactgcaccgagcggcaggccaacttcctgggcaagatttggcccagcaacaagggcaggcccggcaacttcctgcagaaccggcccgagcccaccgcccctcccgaggaaagcttccggttcggcgaggaaaccaccacccccagccagaagcaggaacccatcgacaaagagatgtaccccctggcctccctgaagagcctgttcggcaacgaccccagctcccagtaatgaattc

SEQ ID NO: 19 - DNA, 1501 bp, Artificial Sequence
  Synthetic Polynucleotide
gtcgacgccaccatgggcgctagggccagcatcctgaggggcggcaagctggacaagtgggagaagatccggctgcggcctggcggcaagaaacactacatgctgaagcacctggtctgggccagccgggagctggaacggttcgccctgaaccccggcctgctggaaaccagcgagggctgcaagcagatcatcaagcagctgcagcccgccctgcagaccggcaccgaggaactgcggagcctgttcaacaccgtggccaccctgtactgcgtgcacgccgagatcgaagtgcgggacaccaaagaggccctggacaagatcgaggaagagcagaacaagagccagcagaaaacccagcaggccaaagaagccgacggcaaggtctcccagaactaccccatcgtgcagaacctgcagggccagatggtgcaccagcccatcagcccccggaccctgaacgcctgggtgaaggtgatcgaggaaaaggccttcagccccgaggtgatccccatgttcaccgccctgagcgagggcgccacaccccaggacctgaacaccatgctgaacaccgtgggcggccaccaggccgccatgcagatgctgaaggacaccatcaacgaggaagccgccgagtgggaccggctgcaccctgtgcacgccggacctgtggcccctggccagatgcgggagcccaggggcagcgacatcgccggcacaaccagcaacctgcaggaacagatcgcctggatgaccagcaacccccccatccccgtgggcgacatctacaagcggtggatcatcctgggcctgaacaagatcgtgcggatgtacagccccacctccatcctggacatcaagcagggccccaaagagcccttccgggactacgtggaccggttcttcaagaccctgcgggccgagcaggccacccaggacgtgaagaactggatgaccgacaccctgctggtgcagaacgccaaccccgactgcaagaccatcctgcgggccctgggccctggagccaccctggaagagatgatgaccgcctgccagggcgtgggcggacccagccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgtggcaaggaagggcaccagatgaaggactgcaccgagcggcaggccaacttcctgggcaagatctggccctcccacaagggcaggcccggcaacttcctgcagagcaggcccgagcccacagcccctcccgccgagagcttccggttcgaggaaaccacccctgcccccaagcaggaacccaaggaccgggagcccctgaccagcctgagaagcctgttcggcagcgaccccctgagccagtaatgaattc

SEQ ID NO: 20 - protein, 500 aa, Artificial Sequence
  Synthetic Polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 21 - protein, 500 aa, Artificial Sequence
  Synthetic Polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKSLRAEQASQEVKNWMTETLLVQNANPDCKTILKALGPAATLEEMMTACQGVGGPGQKARLMAEALKEALAPVPIPFAAAQQRGPRKPIKCWNCGKEGHSARQCRAPRRQGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 22 - protein, 498 aa, Artificial Sequence
  Synthetic Polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDLQHPQPAPQQGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGQKARLMAEALKEALAPVPIPFAAAQQRGPRKPIKCWNCGKEGHSARQCRAPRRQGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 23 - protein, 500 aa, Artificial Sequence
  Synthetic Polypeptide
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQ

SEQ ID NO: 24 - protein, 491 aa, Artificial Sequence
  Synthetic Polypeptide
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQ

SEQ ID NO: 25 - protein, 500 aa, Artificial Sequence
  Synthetic Polypeptide
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGQKARLMAEALKEALAPVPIPFAAAQQRGPRKPIKCWNCGKEGHSARQCRAPRRQGCWKCGKEGHQMKDCTERQANFLGKIWPSNKGRPGNFLQNRPEPTAPPEESFRFGEETTTPSQKQEPIDKEMYPLASLKSLFGNDPSSQ

SEQ ID NO: 26 - protein, 493 aa, Artificial Sequence
  Synthetic Polypeptide
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSQKARLMAEALKEALAPVPIPFAAAQQRGPRKPIKCWNCGKEGHSARQCRAPRRQGCWKCGKEGHQMKDCTERQANFLGKIWPSHKGRPGNFLQSRPEPTAPPAESFRFEETTPAPKQEPKDREPLTSLRSLFGSDPLSQ

SEQ ID NO: 27 - DNA, 1500 bp, Artificial Sequence; identical to SEQ ID NO: 14
  Synthetic Polynucleotide

SEQ ID NO: 28 - DNA, 1506 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaagatggaccacgtgatggccaagtgcccggaccgccaggcgggttttttaggccttggtccatggggaaagaagccccgcaatttccccatggctcaagtgcatcaggggctgatgccaacagccccaccagaagagagcttcaggtttggggaagagacaacaactccctctcagaagcaggagccgatagacaaggaactgtatcctttagcttccctcagatcactctttggcagcgacccctcgtcacaa

SEQ ID NO: 29 - DNA, 1497 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccacagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaatag

SEQ ID NO: 30 - protein, 500 aa, Artificial Sequence
  Synthetic Polypeptide
MGARASVLSGGELDRWEKIRLRPGGKKKYKLKHIVWASRELERFAVNPGLLETSEGCRQILGQLQPSLQTGSEELKSLYNTVCVLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKSLRAEQTDAAVKNWMTQTLLVQNANPDCKTILKALGPAATLEEMMTACQGVGGPGQKARLMAEALKEALAPVPIPFAAAQQRGPRKPIKCWNCGKEGHSARQCRAPRRQGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 31 - protein, 502 aa, Artificial Sequence
  Synthetic Polypeptide
MGARASVLSGGELDRWEKIRLRPGGKKKYKLKHIVWASRELERFAVNPGLLETSEGCRQILGQLQPSLQTGSEELKSLYNTVCVLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKSLRAEQTDAAVKNWMTQTLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKMDHVMAKCPDRQAGFLGLGPWGKKPRNFPMAQVHQGLMPTAPPEESFRFGEETTTPSQKQEPIDKELYPLASLRSLFGSDPSSQ

SEQ ID NO: 32 - protein, 498 aa, Artificial Sequence; identical to SEQ ID NO: 22
  Synthetic Polypeptide

SEQ ID NO: 33 - protein, 510 aa, Artificial Sequence
  Synthetic Polypeptide
MGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKPIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESREKPYKEVTEDLLHLNSLFGGDQ

SEQ ID NO: 34 - protein, 501 aa, Artificial Sequence
  Synthetic Polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQR

SEQ ID NO: 35 - DNA, 1530 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 36 - DNA, 1530 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 37 - DNA, 1503 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 38 - DNA, 1506 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 39 - DNA, 1533 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 40 - DNA, 1506 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 41 - DNA, 4541 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatagggggccagctgaaggaggccctgctggacaccggcgccgacgacaccgtgctggaggagatgaacctgcccggccgctggaagcccaagatgatcggcggcatcggcggcttcatcaaggtgggccagtacgaccagatcctgatcgagatctgcggccacaaggccatcggcaccgtgctggtgggccccacccccgtgaacatcatcggccgcaacctgctgacccagatcggctgcaccctgaacttccccatcagccccatcgagaccgtgcccgtgaagctgaagcccggcatggacggccccaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggcgcaagctggtggacttccgcgagctgaacaagcgcacccaggacttctgggaggtgcagctgggcatcccccaccccgccggcctgaagcagaagaagagcgtgaccgtgctggacgtgggcgacgcctacttcagcgtgcccctggacaaggacttccgcaagtacaccgccttcaccatccccagcatcaacaacgagacccccggcatccgctaccagtacaacgtgctgccccagggctggaagggcagccccgccatcttccagtgcagcatgaccaagatcctggagcccttccgcaagcagaaccccgacatcgtgatctaccagtacatggaccacctgtacgtgggcagcgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggacagctggaccgtgaacgacatccagaagctggtgggcaagctgaactgggccagccagatctacgccggcatcaaggtgcgccagctgtgcaagctgctgcgcggcaccaaggccctgaccgaggtggtgcccctgaccgaggaggccgagctggagctggccgagaaccgcgagatcctgaaggagcccgtgcacggcgtgtactacgaccccagcaaggacctgatcgccgagatccagaagcagggccagggccagtggacctaccagatctaccaggagcccttcaagaacctgaagaccggcaagtacgcccgcatgaagggcgcccacaccaacgacgtgaagcagctgaccgaggccgtgcagaagatcgccaccgagagcatcgtgatctggggcaagacccccaagttcaagctgcccatccagaaggagacctgggaggcctggtggaccgagtactggcaggccacctggatccccgagtgggagttcgtgaacaccccccccctggtgaagctgtggtaccagctggagaaggagcccatcatcggcgccgagaccttctacgtggacggcgccgccaaccgcgagaccaagctgggcaaggccggctacgtgaccgaccgcggccgccagaaggtggtgcccctgaccgacaccaccaaccagaagaccgagctgcaggccatccacctggccctgcaggacagcggcctggaggtgaacatcgtgaccgacagccagtacgccctgggcatcatccaggcccagcccgacaagagcgagagcgagctggtgagccagatcatcgagcagctgatcaagaaggagaaggtgtacctggcctgggtgcccgcccacaagggcatcggcggcaacgagcaggtggacggcctggtgagcgccggcatccgcaaggtgctgttcctggacggcatcgacaaggcccaggaggagcacgagaagtaccacagcaactggcgcgccatggccagcgacttcaacctgccccccgtggtggccaaggagatcgtggccagctgcgacaagtgccagctgaagggcgaggccatgcacggccaggtggactgcagccccggcatctggcagctggcatgcacccacctggagggcaaggtgatcctggtggccgtgcacgtggccagcggctacatcgaggccgaggtgatccccgccgagaccggccaggagaccgcctacttcctgctgaagctggccggccgctggcccgtgaagaccgtgcacaccgacaacggcagcaacttcaccagcaccaccgtgaaggccgcctgctggtgggccggcatcaagcaggagttcggcatcccctacaacccccagagccagggcgtgatcgagagcatgaacaaggagctgaagaagatcatcggccaggtgcgcgaccaggccgagcacctgaagaccgccgtgcagatggccgtgttcatccacaacttcaagcgcaagggcggcatcggcggctacagcgccggcgagcgcatcgtggacatcatcgccaccgacatccagaccaaggagctgcagaagcagatcaccaagatccagaacttccgcgtgtactaccgcgacagccgcgaccccgtgtggaagggccccgccaagctgctgtggaagggcgagggcgccgtggtgatccaggacaacagcgacatcaaggtggtgccccgccgcaaggccaagatcatccgcgactacggcaagcagatggccggcgacgactgcgtggccagccgccaggacgaggactaggaatt

SEQ ID NO: 42 - DNA, 1533 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 43 - DNA, 1533 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 44 - DNA, 1506 bp, Artificial Sequence
  Synthetic Polynucleotide
atgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 45 - DNA, 1503 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 46 - DNA, 1530 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 47 - DNA, 1509 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatag

SEQ ID NO: 48 - DNA, 1509 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatag

SEQ ID NO: 49 - DNA, 5874 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 50 - DNA, 5874 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 51 - DNA, 5847 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 52 - DNA, 5850 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 53 - DNA, 5877 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 54 - DNA, 5850 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 55 - DNA, 8880 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatagggggccagctgaaggaggccctgctggacaccggcgccgacgacaccgtgctggaggagatgaacctgcccggccgctggaagcccaagatgatcggcggcatcggcggcttcatcaaggtgggccagtacgaccagatcctgatcgagatctgcggccacaaggccatcggcaccgtgctggtgggccccacccccgtgaacatcatcggccgcaacctgctgacccagatcggctgcaccctgaacttccccatcagccccatcgagaccgtgcccgtgaagctgaagcccggcatggacggccccaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggcgcaagctggtggacttccgcgagctgaacaagcgcacccaggacttctgggaggtgcagctgggcatcccccaccccgccggcctgaagcagaagaagagcgtgaccgtgctggacgtgggcgacgcctacttcagcgtgcccctggacaaggacttccgcaagtacaccgccttcaccatccccagcatcaacaacgagacccccggcatccgctaccagtacaacgtgctgccccagggctggaagggcagccccgccatcttccagtgcagcatgaccaagatcctggagcccttccgcaagcagaaccccgacatcgtgatctaccagtacatggaccacctgtacgtgggcagcgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggacagctggaccgtgaacgacatccagaagctggtgggcaagctgaactgggccagccagatctacgccggcatcaaggtgcgccagctgtgcaagctgctgcgcggcaccaaggccctgaccgaggtggtgcccctgaccgaggaggccgagctggagctggccgagaaccgcgagatcctgaaggagcccgtgcacggcgtgtactacgaccccagcaaggacctgatcgccgagatccagaagcagggccagggccagtggacctaccagatctaccaggagcccttcaagaacctgaagaccggcaagtacgcccgcatgaagggcgcccacaccaacgacgtgaagcagctgaccgaggccgtgcagaagatcgccaccgagagcatcgtgatctggggcaagacccccaagttcaagctgcccatccagaaggagacctgggaggcctggtggaccgagtactggcaggccacctggatccccgagtgggagttcgtgaacaccccccccctggtgaagctgtggtaccagctggagaaggagcccatcatcggcgccgagaccttctacgtggacggcgccgccaaccgcgagaccaagctgggcaaggccggctacgtgaccgaccgcggccgccagaaggtggtgcccctgaccgacaccaccaaccagaagaccgagctgcaggccatccacctggccctgcaggacagcggcctggaggtgaacatcgtgaccgacagccagtacgccctgggcatcatccaggcccagcccgacaagagcgagagcgagctggtgagccagatcatcgagcagctgatcaagaaggagaaggtgtacctggcctgggtgcccgcccacaagggcatcggcggcaacgagcaggtggacggcctggtgagcgccggcatccgcaaggtgctgttcctggacggcatcgacaaggcccaggaggagcacgagaagtaccacagcaactggcgcgccatggccagcgacttcaacctgccccccgtggtggccaaggagatcgtggccagctgcgacaagtgccagctgaagggcgaggccatgcacggccaggtggactgcagccccggcatctggcagctggcatgcacccacctggagggcaaggtgatcctggtggccgtgcacgtggccagcggctacatcgaggccgaggtgatccccgccgagaccggccaggagaccgcctacttcctgctgaagctggccggccgctggcccgtgaagaccgtgcacaccgacaacggcagcaacttcaccagcaccaccgtgaaggccgcctgctggtgggccggcatcaagcaggagttcggcatcccctacaacccccagagccagggcgtgatcgagagcatgaacaaggagctgaagaagatcatcggccaggtgcgcgaccaggccgagcacctgaagaccgccgtgcagatggccgtgttcatccacaacttcaagcgcaagggcggcatcggcggctacagcgccggcgagcgcatcgtggacatcatcgccaccgacatccagaccaaggagctgcagaagcagatcaccaagatccagaacttccgcgtgtactaccgcgacagccgcgaccccgtgtggaagggccccgccaagctgctgtggaagggcgagggcgccgtggtgatccaggacaacagcgacatcaaggtggtgccccgccgcaaggccaagatcatccgcgactacggcaagcagatggccggcgacgactgcgtggccagccgccaggacgaggactaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 56 - DNA, 5877 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 57 - DNA, 5877 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 58 - DNA, 5850 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 59 - DNA, 5847 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 60 - DNA, 5874 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 61 - DNA, 5886 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagataaacttaagcttatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggtaccgagctcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 62 - DNA, 5886 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagataaacttaagcttatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggtaccgagctcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 63 - DNA, 5886 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagataaacttaagcttatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggtaccgagctcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 64 - DNA, 5847 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 65 - DNA, 5841 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccacagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 66 - DNA, 5845 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccaccatgggcgccagggccagcgtgctgtctggcggcgagctggacagatgggagaagatccggctgcggcctggcggcaagaagaagtaccggctgaagcacatcgtgtgggccagccgggagctggaacggttcgccgtgaaccccggcctgctggaaaccagcgagggctgccggcagatcctgggccagctgcagcccagcctgcagaccggcagcgaggaactgcggagcctgtacaacaccgtggccaccctgtactgcgtgcaccagcggatcgagatcaaggacaccaaagaggccctggaaaagatcgaggaagagcagaacaagtccaagaagaaggcccagcaggctgccgccgacaccggcaacagcagccaggtgtcccagaactaccccatcgtgcagaacatccagggccagatggtgcaccaggccatcagcccccggaccctgaacgcctgggtgaaggtggtggaggaaaaggccttcagccccgaggtgatccccatgttcagcgccctgagcgagggcgccacaccccaggacctgaacaccatgctgaacaccgtgggcggccaccaggccgccatgcagatgctgaaagagaccatcaacgaggaagccgccgagtgggacagagtgcaccccgtgcacgccggacctatcgcccctggccagatgcgggagcccaggggcagcgacatcgccggcacaaccagcacactgcaggaacagatcggctggatgaccaacaacccccccatccccgtgggcgagatctacaagcggtggatcatcctgggcctgaacaagatcgtgcggatgtacagccccgtgagcatcctggacatccggcagggccccaaagagcccttccgggactacgtggaccggttctacaagaccctgcgggccgagcaggccagccaggacgtgaagaactggatgaccgagaccctgctggtgcagaacgccaaccccgactgcaagaccatcctgaaggccctgggccctgccgccaccctggaagagatgatgaccgcctgccagggcgtgggcggacctggccacaaggcccgggtgctggccgaggccatgagccaggtgaccaacagcgccaccatcatgatgcagcggggcaacttccggaaccagagaaagaccgtgaagtgcttcaactgcggcaaagagggccacatcgccaagaactgcagggcccccaggaagaagggctgctggaagtgtggcaaggaagggcaccagatgaaggactgcaccgagcggcaggccaacttcctgggcaagatttggcccagcaacaagggcaggcccggcaacttcctgcagaaccggcccgagcccaccgcccctcccgaggaaagcttccggttcggcgaggaaaccaccacccccagccagaagcaggaacccatcgacaaagagatgtaccccctggcctccctgaagagcctgttcggcaacgaccccagctcccagtaatgaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 67 - DNA, 5833 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccaccatgggcgctagggccagcatcctgaggggcggcaagctggacaagtgggagaagatccggctgcggcctggcggcaagaaacactacatgctgaagcacctggtctgggccagccgggagctggaacggttcgccctgaaccccggcctgctggaaaccagcgagggctgcaagcagatcatcaagcagctgcagcccgccctgcagaccggcaccgaggaactgcggagcctgttcaacaccgtggccaccctgtactgcgtgcacgccgagatcgaagtgcgggacaccaaagaggccctggacaagatcgaggaagagcagaacaagagccagcagaaaacccagcaggccaaagaagccgacggcaaggtctcccagaactaccccatcgtgcagaacctgcagggccagatggtgcaccagcccatcagcccccggaccctgaacgcctgggtgaaggtgatcgaggaaaaggccttcagccccgaggtgatccccatgttcaccgccctgagcgagggcgccacaccccaggacctgaacaccatgctgaacaccgtgggcggccaccaggccgccatgcagatgctgaaggacaccatcaacgaggaagccgccgagtgggaccggctgcaccctgtgcacgccggacctgtggcccctggccagatgcgggagcccaggggcagcgacatcgccggcacaaccagcaacctgcaggaacagatcgcctggatgaccagcaacccccccatccccgtgggcgacatctacaagcggtggatcatcctgggcctgaacaagatcgtgcggatgtacagccccacctccatcctggacatcaagcagggccccaaagagcccttccgggactacgtggaccggttcttcaagaccctgcgggccgagcaggccacccaggacgtgaagaactggatgaccgacaccctgctggtgcagaacgccaaccccgactgcaagaccatcctgcgggccctgggccctggagccaccctggaagagatgatgaccgcctgccagggcgtgggcggacccagccacaaggcccgggtgctggccgaggccatgagccagaccaacagcaccatcctgatgcagcggagcaacttcaagggcagcaagcggatcgtgaagtgcttcaactgcggcaaagagggccacatcgcccggaactgcagggcccccaggaagaagggctgctggaagtgtggcaaggaagggcaccagatgaaggactgcaccgagcggcaggccaacttcctgggcaagatctggccctcccacaagggcaggcccggcaacttcctgcagagcaggcccgagcccacagcccctcccgccgagagcttccggttcgaggaaaccacccctgcccccaagcaggaacccaaggaccgggagcccctgaccagcctgagaagcctgttcggcagcgaccccctgagccagtaatgattcacgtaagggcgaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 68 - DNA, 5845 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccaccatgggcgccagggccagcgtgctgtctggcggcgagctggacagatgggagaagatccggctgcggcctggcggcaagaagaagtaccggctgaagcacatcgtgtgggccagccgggagctggaacggttcgccgtgaaccccggcctgctggaaaccagcgagggctgccggcagatcctgggccagctgcagcccagcctgcagaccggcagcgaggaactgcggagcctgtacaacaccgtggccaccctgtactgcgtgcaccagcggatcgagatcaaggacaccaaagaggccctggaaaagatcgaggaagagcagaacaagtccaagaagaaggcccagcaggctgccgccgacaccggcaacagcagccaggtgtcccagaactaccccatcgtgcagaacatccagggccagatggtgcaccaggccatcagcccccggaccctgaacgcctgggtgaaggtggtggaggaaaaggccttcagccccgaggtgatccccatgttcagcgccctgagcgagggcgccacaccccaggacctgaacaccatgctgaacaccgtgggcggccaccaggccgccatgcagatgctgaaagagaccatcaacgaggaagccgccgagtgggacagagtgcaccccgtgcacgccggacctatcgcccctggccagatgcgggagcccaggggcagcgacatcgccggcacaaccagcacactgcaggaacagatcggctggatgaccaacaacccccccatccccgtgggcgagatctacaagcggtggatcatcctgggcctgaacaagatcgtgcggatgtacagccccgtgagcatcctggacatccggcagggccccaaagagcccttccgggactacgtggaccggttctacaagaccctgcgggccgagcaggccagccaggacgtgaagaactggatgaccgagaccctgctggtgcagaacgccaaccccgactgcaagaccatcctgaaggccctgggccctgccgccaccctggaagagatgatgaccgcctgccagggcgtgggcggacctggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgtggcaaggaagggcaccagatgaaggactgcaccgagcggcaggccaacttcctgggcaagatttggcccagcaacaagggcaggcccggcaacttcctgcagaaccggcccgagcccaccgcccctcccgaggaaagcttccggttcggcgaggaaaccaccacccccagccagaagcaggaacccatcgacaaagagatgtaccccctggcctccctgaagagcctgttcggcaacgaccccagctcccagtaatgaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 69 - DNA, 5824 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccaccatgggcgctagggccagcatcctgaggggcggcaagctggacaagtgggagaagatccggctgcggcctggcggcaagaaacactacatgctgaagcacctggtctgggccagccgggagctggaacggttcgccctgaaccccggcctgctggaaaccagcgagggctgcaagcagatcatcaagcagctgcagcccgccctgcagaccggcaccgaggaactgcggagcctgttcaacaccgtggccaccctgtactgcgtgcacgccgagatcgaagtgcgggacaccaaagaggccctggacaagatcgaggaagagcagaacaagagccagcagaaaacccagcaggccaaagaagccgacggcaaggtctcccagaactaccccatcgtgcagaacctgcagggccagatggtgcaccagcccatcagcccccggaccctgaacgcctgggtgaaggtgatcgaggaaaaggccttcagccccgaggtgatccccatgttcaccgccctgagcgagggcgccacaccccaggacctgaacaccatgctgaacaccgtgggcggccaccaggccgccatgcagatgctgaaggacaccatcaacgaggaagccgccgagtgggaccggctgcaccctgtgcacgccggacctgtggcccctggccagatgcgggagcccaggggcagcgacatcgccggcacaaccagcaacctgcaggaacagatcgcctggatgaccagcaacccccccatccccgtgggcgacatctacaagcggtggatcatcctgggcctgaacaagatcgtgcggatgtacagccccacctccatcctggacatcaagcagggccccaaagagcccttccgggactacgtggaccggttcttcaagaccctgcgggccgagcaggccacccaggacgtgaagaactggatgaccgacaccctgctggtgcagaacgccaaccccgactgcaagaccatcctgcgggccctgggccctggagccaccctggaagagatgatgaccgcctgccagggcgtgggcggacccagccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgtggcaaggaagggcaccagatgaaggactgcaccgagcggcaggccaacttcctgggcaagatctggccctcccacaagggcaggcccggcaacttcctgcagagcaggcccgagcccacagcccctcccgccgagagcttccggttcgaggaaaccacccctgcccccaagcaggaacccaaggaccgggagcccctgaccagcctgagaagcctgttcggcagcgaccccctgagccagtaatgaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 70 - DNA, 1530 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 71 - DNA, 5874 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 72 - DNA, 1524 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 73 - DNA, 5868 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 74 - DNA, 1524 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 75 - DNA, 5868 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 76 - DNA, 1503 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaatag

SEQ ID NO: 77 - DNA, 5847 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 78 - DNA, 1506 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgtgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaagatggaccacgtgatggccaagtgcccggaccgccaggcgggttttttaggccttggtccatggggaaagaagccccgcaatttccccatggctcaagtgcatcaggggctgatgccaacagccccaccagaagagagcttcaggtttggggaagagacaacaactccctctcagaagcaggagccgatagacaaggaactgtatcctttagcttccctcagatcactctttggcagcgacccctcgtcacaa

SEQ ID NO: 79 - DNA, 5850 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgtgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaagatggaccacgtgatggccaagtgcccggaccgccaggcgggttttttaggccttggtccatggggaaagaagccccgcaatttccccatggctcaagtgcatcaggggctgatgccaacagccccaccagaagagagcttcaggtttggggaagagacaacaactccctctcagaagcaggagccgatagacaaggaactgtatcctttagcttccctcagatcactctttggcagcgacccctcgtcacaagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 80 - DNA, 1524 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccacagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 81 - DNA, 5868 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccacagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 82 - DNA, 1497 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccgcagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 83 - DNA, 5841 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccgcagcagggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 84 - DNA, 1506 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccgcagcagggccagctgcgcgagcccagcggcagcgacatcgccggcaccaccagcagcgtggacgagcagatccagtggatgtaccgccagcagaaccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatag

SEQ ID NO: 85 - DNA, 5850 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccgcagcagggccagctgcgcgagcccagcggcagcgacatcgccggcaccaccagcagcgtggacgagcagatccagtggatgtaccgccagcagaaccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 86 - DNA, 1527 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccgcagcagggccagctgcgcgagcccagcggcagcgacatcgccggcaccaccagcagcgtggacgagcagatccagtggatgtaccgccagcagaaccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 87 - DNA, 5871 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggacctgcagcacccgcagcccgcgccgcagcagggccagctgcgcgagcccagcggcagcgacatcgccggcaccaccagcagcgtggacgagcagatccagtggatgtaccgccagcagaaccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 88 - DNA, 1536 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 89 - DNA, 5880 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 90 - DNA, 1524 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 91 - DNA, 5868 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 92 - DNA, 1533 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 93 - DNA, 5877 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 94 - DNA, 1509 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 95 - DNA, 5853 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataagaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 96 - DNA, 1509 bp, Artificial Sequence
  Synthetic Polynucleotide
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggagagcttcaggtttggggaagagacaacaactccctctcagaagcaggagccgatagacaaggaactgtatcctttagcttccctcagatcactctttggcagcgacccctcgtcacaatag

SEQ ID NO: 97 - DNA, 5853 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacgccgccaccatgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgaagagcctgtacaacaccgtgtgcgtcctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagtccctgcgcgccgagcagaccgacgcggcggtgaagaactggatgacccagaccctgctggtgcagaacgccaaccccgactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggttttttaggccttggtccatggggaaagaagccccgcaatttccccatggctcaagtgcatcaggggctgatgccaactgctcccccagaggagagcttcaggtttggggaagagacaacaactccctctcagaagcaggagccgatagacaaggaactgtatcctttagcttccctcagatcactctttggcagcgacccctcgtcacaataggaattctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 98 - DNA, 2547 bp, Artificial Sequence
  Synthetic Polynucleotide
atgcgggtgaagggcatcaggaagaactaccagcacctgtggagatggggaacaatgctgctgggcatgctgatgatctgttctgccgccgaacagctgtgggtgacagtgtactatggcgtgcccgtgtggaaagaggccaccaccaccctgttttgtgccagcgacgccaaggcctatgacaccgaggtgcacaatgtgtgggccactcatgcctgtgtgcccaccgatcccaatcctcaggaagtggtcctgggcaacgtgaccgagaacttcaacatgtggaagaacaacatggtggagcagatgcacgaggacatcatcagcctgtgggacgagtctctgaagccctgtgtgaagctgacccctctgtgcgtgaccctgaactgcaccgacctgagaaacgccaccaacaccacaagctctagctgggagaccatggaaaagggcgagatcaagaactgcagcttcaacatcaccacctccatccgggacaaggtgcagaaagagtacgccctgttctacaagctggacgtggtgcccatcgacaacgacaacaccagctaccggctgatcaactgcaacaccagcgtgatcacccaggcctgccctaaggtgtccttcgagcccatccctatccactattgcgcccctgccggctttgccatcctgaagtgcaacgacaagaagttcaacggcaccggcccttgcaagaatgtgtccaccgtgcagtgtacacacggcatcagacctgtggtgtccacccagctcctgctgaatggctctctggccgaggaagaggtggtgatcagaagcgagaactttaccaacaacgccaagaccatcatcgtgcagctgaacgagagcgtggagatcaattgcacccggcccaacaacaatacccggaagagcatccacattggccctggccaggccttttatgccaccggcgacatcatcggcgatattcggcaggcccactgcaatatcagccgggccaagtggaataacaccctgaagcagatcgtgatcaagctgcgggagcagttcggcaacaagaccatcgtgttcaatcagagcagcggcggagatcctgagatcgtgatgcacagcttcaactgtggcggcgagttcttctactgcaacacaacccagctgttcaacagcacctggaacgtgaatggcacctggaatggcaccggcagcgagaatatcaccctgccctgccggatcaagcagattgtgaacatgtggcaggaagtgggcaaagccatgtacgcccctcctatcagaggccagatccggtgcagcagcaatatcaccggcctgctgctgacaagagatggcggcaacaacaacagcaccaacgagacctttagacctggcggcggagacatgagggacaattggcggagcgagctgtacaagtacaaggtggtgaagatcgaacctctgggcgtggctcctaccaaggccaagcggagagtggtgcagagggaaaaaagagccgtgggcctgggagctgtgtttctgggctttctgggaacagccggctctacaatgggagccgccagcctgacactgacagtgcaggccagactgctgctgtctggcatcgtgcagcagcagaacaacctgctgagagccattgaagcccagcagcacatgctgcagctgacagtgtggggcattaagcagctgcaggctagagtgctggccgtggagagatacctgaaggatcagcagctcctgggactgtggggctgtagcggcaagctgatctgcaccaccaacgtgccttggaacagcagctggtccaacaagagccaggaagagatctggaacaacatgacctggatggaatgggagcgggagatcgacaattacaccggcctgatctacaccctgatcgaggaaagccagaaccagcaggaaaagaacgagcaggaactgctggaactggataagtgggccagcctgtggaactggttcgacatcaccaactggctgtggtacatcaagatcttcatcatgatcgtgggcggcctgatcggcctgagaatcgtgttcgccgtgctgtccatcatcaacagagtgaggcagggctactctcctctgtctctgcagacactgctgcctgctcctagaggccctgatagacccgagggcatcgaagaagaaggcggcgagcagggcagagatagaagcatccggctggtgaacggctttctggccctgatctgggacgatctgcggaacctgtgcctgttcagctaccacaggctgagggatctgctgctgatcgtgaccagaattgtggagctgctggggagaagaggatgggaggccctgaagtactggtggaacctgctgcagtactggtcccaggaactgaagaatagcgccgtgagcctgctgaatgccacagccattgccgtggccgagggcacagatagagtgatcgaggtggcccagagagcttggagagccatcctgcacatccccagaagaatccggcagggactggaaagggctctgctgtgatga

SEQ ID NO: 99 - protein, 847 aa, Artificial Sequence
  Synthetic Polypeptide
MRVKGIRKNYQHLWRWGTMLLGMLMICSAAEQLWVTVYYGVPVWKEATTTLFCASDAKAYDTEVHNVWATHACVPTDPNPQEVVLGNVTENFNMWKNNMVEQMHEDIISLWDESLKPCVKLTPLCVTLNCTDLRNATNTTSSSWETMEKGEIKNCSFNITTSIRDKVQKEYALFYKLDVVPIDNDNTSYRLINCNTSVITQACPKVSFEPIPIHYCAPAGFAILKCNDKKFNGTGPCKNVSTVQCTHGIRPVVSTQLLLNGSLAEEEVVIRSENFTNNAKTIIVQLNESVEINCTRPNNNTRKSIHIGPGQAFYATGDIIGDIRQAHCNISRAKWNNTLKQIVIKLREQFGNKTIVFNQSSGGDPEIVMHSFNCGGEFFYCNTTQLFNSTWNVNGTWNGTGSENITLPCRIKQIVNMWQEVGKAMYAPPIRGQIRCSSNITGLLLTRDGGNNNSTNETFRPGGGDMRDNWRSELYKYKVVKIEPLGVAPTKAKRRVVQREKRAVGLGAVFLGFLGTAGSTMGAASLTLTVQARLLLSGIVQQQNNLLRAIEAQQHMLQLTVWGIKQLQARVLAVERYLKDQQLLGLWGCSGKLICTTNVPWNSSWSNKSQEEIWNNMTWMEWEREIDNYTGLIYTLIEESQNQQEKNEQELLELDKWASLWNWFDITNWLWYIKIFIMIVGGLIGLRIVFAVLSIINRVRQGYSPLSLQTLLPAPRGPDRPEGIEEEGGEQGRDRSIRLVNGFLALIWDDLRNLCLFSYHRLRDLLLIVTRIVELLGRRGWEALKYWWNLLQYWSQELKNSAVSLLNATAIAVAEGTDRVIEVAQRAWRAILHIPRRIRQGLERALL

SEQ ID NO: 100 - DNA, 2595 bp, Artificial Sequence
  Synthetic Polynucleotide
atgcgggtgaaagaaacccagatgaactggcccaatctgtggaagtggggcacactgatcctgggcctggtgatcatctgcagcgccagcgataatctgtgggtgaccgtgtactatggcgtgcctgtgtggaaagaggccaagaccaccctgttttgtgccagcgatgccaaggcctacgagaaagaggtgcacaacatctgggccacacacgcctgtgtgcccaccgatcccaaccctcaggaaatccacctggaaaacgtgaccgagaacttcaacatgtggaagaacgacatggtggaccagatgcacgaggacatcatcagcctgtgggaccagtctctgaagccctgtgtgaagctgacccctctgtgcgtgaccctgaactgcaccaacgccaacctgaccaatggcagcagcaagaccaacgtgagcaacatcatcggcaacatcaccgacgaagtgcggaactgcagcttcaacatgaccaccgagctgcgggacaagaaacagaaggtgcacgccctgttctacaagctggacatcgtgcccatcgaggacaacagcaacagcagcgagtaccggctgatcaactgcaataccagcgccatcacccaggcctgtcccaaggtgtccttcgaccccatccctatccactattgtgcccctgccggctacgccatcctgaagtgcaacaacaagaccttcaacggcaccggcccctgtacaaatgtgtccaccgtgcagtgtacacacggcatcaagcctgtggtgtccacccagctgctgtttaatggcagcctggccgaggaagagatcatcatccggtccgagaatctgaccaacaacgccaagaccatcatcgtgcacctgaacaagagcgtggagatcaattgcacccggcccagcaacaacacccggaagagcatcagaatcggccctggccagaccttttacgccaccggcgacatcattggcgacatccggaaggcctactgcgagatcaacggcacaaagtggaacgagaccctgaagcaggtggccaagaagctgaaagagcacttcaacaacaaaaccatcatcttcaacagcagcagcggcggagatctggaaatcaccacccacagcttcaactgcaggggcgagttcttctactgtaacaccagcggcctgttcaatagcacctggtccctgaatagcagcgcccctgacgacaccgagagcaacgataccatcaccctgccctgccggatcaagcagatcatcaatatgtggcaggaagtgggcagagccatgtatgcccctcccatcgccggcaatatcacctgcaagtccaatatcaccggcctgatcctgacaagagatggcggcaacaacaaagagaccaacgagaccgagacctttagacctggcggcggaaacatgaaggacaactggcggagcgagctgtacaagtacaaggtggtggagattaagcctctgggcgtggctcctaccagagccaagcggagagtggtggagagggaaaaaagagccgtgggcatcggagccgtgtttctgggctttctgggagccgctggatctacaatgggagccgccagcatcacactgacagtgcaggccagacagctgctctctggcatcgtgcagcagcagagcaatctgctgagagccatcgaagcccagcagcatctgctgcagctgacagtgtggggcatcaagcagctgcagaccagagtgctggccatcgagagatacctgaaggatcagcagctcctgggcatctggggctgtagcggcaagctgatctgtacaaccgccgtgccttggaacgccagctggtccaacaagagcctgaacgagatctgggacaacatgacctggatgcagtgggaccgggagatcagcaactacaccaacaccatctaccggctgctggaagatagccagaaccagcaggaaaagaacgagcaggacctgctggctctggataaatgggccagcctgtggagctggttcgacatcagcaactggctgtggtacatccggatcttcatcatgatcgtgggcggcctgatcggcctgagaatcatcttcgccgtgctgtccatcgtgaacagagtgagacagggctacagccctctgagctttcagaccctgacccccaatcctagaggccctgacagactgggcagaatcgaggaagagggcggcgagcaggacagagacagatccatcaggctggtgtctggatttctggccctggcctgggatgatctgagaagcctgtgcctgttcagctaccaccggctgagggactttatcctgatcgccgccagaacagtggaactgctgggccacagctctctgaaaggcctgagactgggctgggagggcctgaaatacctgggcagcctggtgcagtattggggcctggaactgaagaagagcgccatcagcctgctggatacaattgccatcgccgtggccgagggcacagataggatcatcgagctgatccagcggatctgccgggccatcaggaacatccccagacggatcagacagggctttgagagggccctgctgtgatga

SEQ ID NO: 101 - protein, 863 aa, Artificial Sequence
  Synthetic Polypeptide
MRVKETQMNWPNLWKWGTLILGLVIICSASDNLWVTVYYGVPVWKEAKTTLFCASDAKAYEKEVHNIWATHACVPTDPNPQEIHLENVTENFNMWKNDMVDQMHEDIISLWDQSLKPCVKLTPLCVTLNCTNANLTNGSSKTNVSNIIGNITDEVRNCSFNMTTELRDKKQKVHALFYKLDIVPIEDNSNSSEYRLINCNTSAITQACPKVSFDPIPIHYCAPAGYAILKCNNKTFNGTGPCTNVSTVQCTHGIKPVVSTQLLFNGSLAEEEIIIRSENLTNNAKTIIVHLNKSVEINCTRPSNNTRKSIRIGPGQTFYATGDIIGDIRKAYCEINGTKWNETLKQVAKKLKEHFNNKTIIFNSSSGGDLEITTHSFNCRGEFFYCNTSGLFNSTWSLNSSAPDDTESNDTITLPCRIKQIINMWQEVGRAMYAPPIAGNITCKSNITGLILTRDGGNNKETNETETFRPGGGNMKDNWRSELYKYKVVEIKPLGVAPTRAKRRVVEREKRAVGIGAVFLGFLGAAGSTMGAASITLTVQARQLLSGIVQQQSNLLRAIEAQQHLLQLTVWGIKQLQTRVLAIERYLKDQQLLGIWGCSGKLICTTAVPWNASWSNKSLNEIWDNMTWMQWDREISNYTNTIYRLLEDSQNQQEKNEQDLLALDKWASLWSWFDISNWLWYIRIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARTVELLGHSSLKGLRLGWEGLKYLGSLVQYWGLELKKSAISLLDTIAIAVAEGTDRIIELIQRICRAIRNIPRRIRQGFERALL

(19) United States
(12) Patent Application Publication
(10) Pub. No.: US 2012/0219583 A1
(43) Pub. Date: Aug. 30, 2012

(54) NUCLEIC ACID SEQUENCES ENCODING EXPANDABLE HIV MOSAIC PROTEINS

(75) Inventors: Gary J. Nabel, Washington, DC (US); Zhi-Yong Yang, Potomac, MD (US); Wing-Pui Kong, Germantown, MD (US); Bette Korber, Los Alamos, NM (US)

(73) Assignees: Los Alamos National Security, LLC, Los Alamos, NM (US); The U.S. of America, as rep. by the Sec., Dept. of HHS, Bethesda, MD (US)

(21) Appl. No.: 13/502,206

(22) PCT Filed: Oct. 15, 2010
(86) PCT No.: PCT/US10/52916

§ 371 Date: May 14, 2012

Related U.S. Application Data
(60) Provisional application No. 61/252,545, filed on Oct. 16, 2009.

(51) Int. Cl. A61K 31/713; C07K 14/00; C12N 15/63; C12N 15/861; B65D 85/00; A61P 37/04; A61P 31/18; C12N 1/19; C12N 5/10; C12N 15/11; C12N 1/21

(52) U.S. Cl. 424/208.1; 536/23.1; 530/350; 435/320.1; 435/252.33; 435/348; 435/254.2; 435/325; 435/358; 435/369; 435/365; 435/366; 206/524.1

(57) ABSTRACT

The invention is directed to a nucleic acid molecule encoding a HIV-1 polypeptide which comprises the nucleic acid sequence of SEQ ID NO: 1, SEQ ID NO: 2, SEQ ID NO: 3, or SEQ ID NO: 4. The invention also provides a method of inducing an immune response against HIV-1 in a mammal.

### CROSS-REFERENCE TO RELATED APPLICATIONS

This patent application claims the benefit of U.S. Provisional Patent Application No. 61/252,545, filed Oct. 16, 2009, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 503,568 Byte ASCII (Text) file named “707017_ST25.txt,” created on Oct. 15, 2010.

### BACKGROUND OF THE INVENTION

The development of an AIDS vaccine has been advanced recently by demonstrations of increased survival and decreased viral load following vaccination with T-cell vaccines in non-human primate models (see, e.g., Kawada et al., J. Virol., 82: 10199-101206 (2008); Letvin et al., Science, 312: 1530-1533 (2006); Matano et al., J. Exp. Med., 199: 1709-1718 (2004); Santra, Proc. Natl. Acad. Sci. USA, 105: 10489-10494 (2008); Wilson et al., J. Virol., 80: 5875-5885 (2006)). Although such vaccines have suggested that T-cells may contribute to the control of HIV viremia in the highly lethal SIVmac251 challenge model, how these results apply to human studies remains uncertain. The major concern regarding the efficacy of HIV vaccines in humans is the extraordinary genetic diversity of the virus. The sequence similarity of HIV-1 Envelope protein (Env) from diverse isolates within a clade can diverge as much as 15%, and between alternative clades can diverge as much as 30% (see, e.g., Gaschen et al., Science, 296: 2354-2360 (2002)). In addition, the diversity of the HIV-1 Gag protein can approach similar levels, particularly in the p17 and p15 regions which are much more diverse than the p24 region (see, e.g., Fischer et al., Nat. Med., 13: 100-106 (2007)), although Gag does not have the extreme localized diversity observed in the highly variable regions of Env (see, e.g., Fischer et al., supra, and Gaschen et al., supra). While viral diversity has been addressed in existing vaccines through the use of envelopes derived from representative viruses in the major clades, increasing knowledge about the genetic diversity of naturally occurring isolates has enabled alternative approaches that enhance population coverage of vaccine-elicited T-cell responses.

Approaches under consideration include the use of ancestral, central or consensus, and “center of the tree” gene sequences (see, e.g., Doria-Rose et al, J. Virol., 79: 11214-11224 (2005); Gaschen et al., supra; Kothe et al., Virology, 352: 438-449 (2006); Santra et al., supra; and Weaver et al., J. Virol., 80: 6745-6756 (2006)). Such gene sequences can be derived using a number of alternative approaches, including the alignment of HIV gene sequences with selection of the most common amino acids at each residue (see, e.g., Gaschen et al., supra; Korber et al., Br. Med. Bull., 58: 19-42 (2001); Kothe et al., Virology, 360: 218-234 (2007); Liao et al., Virology, 353: 268-282 (2006); Novitsky et al., J. Virol., 76: 5435-5451 (2002); Weaver et al., supra), modeling the most recent common ancestor of diverging viruses in a vaccine target population (see, e.g., Doria-Rose et al., supra; Gaschen et al., supra; Kothe et al., Virology, 352: 438-449 (2006); Weaver et al., supra), or modeling the sequence at the center of the phylogenetic tree (see, e.g., Rolland et al., J. Virol., 81: 8507-8514 (2007)). Peptides based on any of these three centralized protein strategies enhance the detection of T-cell responses in a natural HIV-1 infection relative to the use of peptides based on natural strains; however, all three strategies produce equivalent results (see, e.g., Frahm et al., AIDS, 22: 447-456 (2008)).

The use of a single HIV-1 group M consensus/ancestral Env sequence has been shown to elicit T-cell responses with greater breadth of cross-reactivity than single natural strains in animal models (see, e.g., Santra et al., supra; Weaver et al., supra). Such central sequences do not exist in nature, and phylogenetic ancestral reconstructions are an approximate model of an ancestral state of the virus (see, e.g., Gao et al., Science, 299: 1517-1518 (2003)). Thus, central sequence strategies have provided evidence that various informatically-derived gene products can elicit immune responses to T-cell epitopes found in diverse circulating strains. While consensus genes have been found to be superior to wild-type genes (see, e.g., Weaver et al., supra; Santra et al., supra), the ability of the most recent informatically-derived HIV-1 gene products (also known as “mosaics”) to elicit immune responses to T-cell epitopes found in diverse circulating strains has not been defined.

Thus, there remains a need for vaccines against HIV-1 which improve, and desirably optimize, coverage of T-cell epitopes. This invention provides nucleic acid sequences for HIV-1 vaccination, as well as methods for using such nucleic acid sequences.

### BRIEF SUMMARY OF THE INVENTION

The invention provides an isolated or purified nucleic acid molecule comprising the nucleic acid sequence of SEQ ID NO: 1, SEQ ID NO: 2, SEQ ID NO: 3, or SEQ ID NO: 4.

### BRIEF DESCRIPTION OF THE SEVERAL VIEWS OF THE DRAWING(S)

FIG. 1A is an alignment of SIV and HIV Gag amino acid sequences (mosaic and non-mosaic) generated as described in Example 1. The approximate domain boundaries for HIV Gag p1, p2, p6, p7, p17, p24, the CypA binding site, Helix 5, 6, 7, and budding motif are indicated. SIV Gag T-cell epitopes KV9, DD13, AL11 also are indicated. Modification regions of N5 (SEQ ID NO: 30) and N6 (SEQ ID NO: 31) are indicated. Boundaries of the regions undergoing HIV/SIV chimeric swapping are indicated by upward and downward arrows.

FIG. 1B (left panel) is a diagram which illustrates nucleic acid sequences encoding HIV/SIV Gag chimeric polypeptides, with HIV sequences regions and SIV regions. SIV Gag T-cell epitopes KV9, DD13, AL11, as well as the reference regions CypA binding site, Helix 5, 6, 7, and budding motif are labeled. FIG. 1B (right panel) is a table showing the percentage of AL11 tetramer positive CD8 T lymphocytes elicited by plasmid constructs containing each nucleic acid sequence. The constructs with elevated T cells responses were selected for further study.

FIGS. 2A and 2B each includes a diagram which illustrates nucleic acid sequences encoding HIV/SIV Gag chimeric polypeptides containing the SIV Gag AL11 CD8 epitope, and a table showing the percentage of AL11 tetramer positive CD8 T lymphocytes by plasmid constructs containing each nucleic acid sequence. The plasmid constructs containing the nucleic acid sequences represented in FIG. 2A were generated based on selected plasmid constructs from Example 1. Their T cell responses were evaluated in mice, and two plasmid constructs were selected for further study. Based on the selected plasmid constructs from FIG. 2A, a third batch of plasmid constructs containing the nucleic acid sequences encoding the HIV/SIV Gag chimeric polypeptides of FIG. 2B were made and evaluated. Plasmid constructs containing the N5 (VRC 4717) and N6 (VRC 4718) nucleic acid sequences (SEQ ID NO: 77 and SEQ ID NO: 79, respectively) elicited the highest and longest-lasting tetramer responses.

FIGS. 3A and 3B are graphs which compare the CD4 (FIG. 3A) and CD8 (FIG. 3B) responses elicited by a set of two mosaic wild-type HIV Gag genes (i.e., mosaic Gag1 (WT) (VRC 4700) (SEQ ID NO: 16)) and mosaic Gag2(WT) (VRC 4704) (SEQ ID NO: 17)) and a set of two N5-modified mosaic Gag (N5) genes (i.e., mosaic Gag1 (N5) (VRC 4701) (SEQ ID NO: 18) and mosaic Gag2(N5) (VRC 4705) (SEQ ID NO: 19)) after an immunization regimen utilizing plasmid constructs containing each of these sequences as a prime, and recombinant adenoviral vector constructs containing each of these sequences as a boost (DNA/rAd). Empty vectors served as controls. The bars show the positive IFN-γ responses. The data represent the mean values of the responses with error bars as the standard deviation. There was one unique CD4 positive peptide, 81-277, in the mosaic Gag(N5) group, and there were two unique CD8 positive peptides, 75-154 and 69-398, from mosaic Gag(N5) immunized mice.

FIG. 4A is a graph which demonstrates an increased subdominant CD4 response to PTE peptides elicited by a set of two mosaic Gag(N5) sequences (VRC 4701 and VRC 4705) (SEQ ID NO: 18 and SEQ ID NO: 19) as compared to a set of two mosaic Gag(WT) sequences (VRC 4700 and VRC 4704) (SEQ ID NO: 16 and SEQ ID NO: 17) in DNA/rAd-immunized B6D2F1/J mice. Intracellular cytokine staining (ICS) of CD4 T cell responses to three subdominant HIV Gag15mer PTE peptides are shown. The three 15-mer PTE peptides sequences and their positions (relative to HXB2 positions) are indicated. Each bar shows the average IFN-γ response from two experiments (error bars indicate standard deviation). Only the mosaic Gag(N5) group elicited a statistically significant CD4 response, as compared to the mosaic Gag(WT) and the control groups. The significance of the cellular responses was calculated using the Student's t test (unpaired; tails=1 (to Control), and tails=2 (to mosaic Gag (WT)) as indicated by the p value.

FIG. 4B is a graph which demonstrates an increased subdominant CD8 response to PTE peptides elicited by a set of two nucleic acid sequences encoding mosaic Gag(N5) proteins (VRC 4701 and VRC 4705) (SEQ ID NO: 18 and SEQ ID NO: 19) as compared to a set of two nucleic acid sequences encoding mosaic Gag(WT) proteins (VRC 4700 and VRC 4704) (SEQ ID NO: 16 and SEQ ID NO: 17) in DNA/rAd immunized B6D2F1/J mice. Intracellular cytokine staining (ICS) of CD8 T cell responses to three subdominant HIV Gag15mer PTE peptides are shown. The three 15-mer PTE peptides sequences and their positions (relative to HXB2 positions) are indicated. Each bar shows the average IFN-γ response from two experiments (error bars indicate standard deviation). Only the mosaic Gag(N5) group elicited a statistically significant CD8 response, as compared to the mosaic Gag(WT) and the control groups. The significance of the cellular responses was calculated using the Student's t test (unpaired; tails=1 (to Control), and tails=2 (to mosaic Gag(WT)) as indicated by the p value.

FIGS. 5A and 5B are graphs which illustrate the CD4 (FIG. 5A) and CD8 (FIG. 5B) responses elicited in mice following administration of two adenoviral vector constructs (rAd), each of which encodes a mosaic Gag(WT) protein or two adenoviral vector constructs, each of which encodes an N5-modified mosaic Gag(N5) protein. Specifically, four adenoviral vector constructs containing the mosaic Gag1 (WT) sequence (VRC 4700) (SEQ ID NO: 16), the mosaic Gag2(WT) sequence (VRC 4704) (SEQ ID NO: 17), the mosaic Gag1(N5) sequence (VRC 4701) (SEQ ID NO: 18), and the mosaic Gag2(N5) sequence (VRC 4705) (SEQ ID NO: 19), respectively. Empty vectors served as controls. Only the ICS positive CD4 and CD8 responses against the PTE peptides referring to a unique Gag position without duplication in position are shown. The bars show the positive IFN-γ responses. The data represent the mean values of the responses with error bars as the standard deviation. There was one unique CD4 positive peptide, 7-259, in the mosaic Gag(N5) group, and there were two unique CD8 positive peptides, 45-348 and 76-354, from mosaic Gag(N5)-immunized mice.

FIG. 6A and FIG. 6B are graphs which illustrate increased subdominant CD4 (FIG. 6A) and CD8 (FIG. 6B) responses to PTE peptides elicited in B6D2F1/J mice immunized with adenoviral vector constructs containing a nucleic acid sequence encoding a mosaic Gag(WT) polypeptide (VRC 4700 and VRC 4704) (SEQ ID NO: 16 or SEQ ID NO: 17) or adenoviral vector constructs containing a nucleic acid sequence encoding a mosaic Gag(N5) polypeptide (VRC 4701 and VRC 4705) (SEQ ID NO: 18 and SEQ ID NO: 19). Intracellular cytokine staining (ICS) of CD4 and CD8 T cell responses to three subdominant HIV Gag15mer PTE peptides are shown. The three 15-mer PTE peptides sequences and their positions (relative to HXB2 positions) are indicated. Each bar shows the average IFN-γ from two experiments (error bars indicate standard deviation). Only the mosaic Gag(N5) group elicited statistically significant CD4 or CD8 responses, as compared to the mosaic Gag(WT) and the control groups. The significance of the cellular responses was calculated using the Student's t test (unpaired; tails=1 (to Control), and tails=2 (to mosaic Gag (WT)) as indicated by the p value.

FIG. 7 is a graph which illustrates the CD4 immunogenicity elicited by administration of an adenoviral vector construct encoding a mosaic Env protein and an adenoviral vector construct encoding an N5-modified Gag protein in mice. Four adenoviral vector constructs were generated containing the following nucleic acid sequences: (i) a first mosaic Env nucleic acid sequence (VRC 5926) (SEQ ID NO: 98), (ii) a second mosaic Env nucleic acid sequence (VRC 5927) (SEQ ID NO: 100), (iii) a first N5-modified mosaic Gag sequence (VRC 4701) (SEQ ID NO: 18), and (iv) a second N5-modified mosaic Gag sequence (VRC 4705) (SEQ ID NO: 19). The adenoviral vector constructs were administered to mice alone or in combination. Empty vectors served as controls. All individual 492 individual 15-mer HIV Env PTEs were grouped into 41 pools (12 peptides per pool), and the individual 320 individual 15-mer HIV Gag PTE were grouped into 32 pools (10 peptides per pool), all of which were tested via ICS stimulation. The bars show the CD4 T cell positive IFN-γ responses to that particular PTE peptide pool. The data represent the mean values of the responses from the two experiments with error bars as the standard deviation.

FIG. 8 is a graph which illustrates the CD8 immunogenicity elicited by administration of an adenoviral vector construct encoding a mosaic Env protein and an adenoviral vector construct encoding an N5-modified Gag protein to mice. Four adenoviral vector constructs were generated containing the following nucleic acid sequences: (i) a first mosaic Env nucleic acid sequence (VRC 5926) (SEQ ID NO: 98), (ii) a second mosaic Env nucleic acid sequence (VRC 5627) (SEQ ID NO: 100), (iii) a first N5-modified mosaic Gag sequence (VRC 4701) (SEQ ID NO: 18), and (iv) a second N5-modified mosaic Gag sequence (VRC 4705) (SEQ ID NO: 19). The adenoviral vector constructs were administered to mice alone or in combination. Empty vectors served as controls. All individual 492 individual 15-mer HIV Env PTEs were grouped into 41 pools (12 peptides per pool), and the individual 320 individual 15-mer HIV Gag PTE were grouped into 32 pools (10 peptides per pool), all of which were tested via ICS stimulation. The bars show the CD8 T cell positive IFN-γ responses to that particular PTE peptide pool. The data represent the mean values of the responses with error bars showing the standard deviation.

FIGS. 9A and 9B are graphs which illustrate Gag protein levels in human CD4 T cells (FIG. 9A) and mouse myoblast C2C12 cells (FIG. 9B) transfected with plasmid constructs encoding wild-type SIV Gag, wild-type HIV Gag (VRC 4401) (SEQ ID NO: 13), N5-modified Gag (HIV-gag-N5) (VRC 4708) (SEQ ID NO: 14), wild-type mosaic Gag1 (VRC 4700) (SEQ ID NO: 16), and N5-modified mosaic Gag1 (VRC 4701) (SEQ ID NO: 18). Cell lysates and supernatants were collected 48 hours post-transfection and the Gag proteins were subjected to quantitative ELISA. Western blot of β-actin served as a quantity control. The data represent the mean values of the three different transfections with error bars as the standard deviation. The significance of the expression difference was calculated using the Student's t test as indicated by the p value.

FIG. 10 is an alignment of the amino acid sequences of clade B wild-type Gag (B Gag(WT)) (VRC 4401) (SEQ ID NO: 13), N5-modified HIV Gag (B Gag(N5)) (VRC 4708) (SEQ ID NO: 14), N6-modified HIV Gag (B Gag(N6)) (VRC 4707) (SEQ ID NO: 15), two wild-type mosaic Gag proteins (i.e., mosaic Gag1(WT) (VRC 4700) (SEQ ID NO: 16) and mosaic Gag2(WT) (VRC 4704) (SEQ ID NO: 17)), and two N5-modified mosaic Gag constructs (mosaic Gag1 (N5) (VRC 4701) (SEQ ID NO: 18) and mosaic Gag2(N5) (VRC 4705) (SEQ ID NO: 19)). The modification regions of N5 and N6 are indicated as boxed regions.

FIGS. 11A and 11B are graphs which illustrate CD4 (FIG. 11A) and CD8 (FIG. 11B) TNF-α responses elicited by mosaic Gag(WT) and N5 modified mosaic Gag(N5) polypeptides after an immunization regimen utilizing, as a prime, a plasmid construct containing the mosaic Gag1(WT) sequence (VRC 4700) (SEQ ID NO: 16), the mosaic Gag2(WT) sequence (VRC 4704) (SEQ ID NO: 17), the mosaic Gag1 (N5) sequence (VRC 4701) (SEQ ID NO: 18), and the mosaic Gag2(N5) sequence (VRC 4705) (SEQ ID NO: 19)), and, as a boost, a recombinant adenoviral vector construct containing SEQ ID NO: 16, SEQ ID NO: 17, SEQ ID NO: 18, or SEQ ID NO: 19. The bars show the positive TNF-α responses with error bars as the standard deviation.

FIGS. 12A and 12B are graphs which illustrate the CD4 (FIG. 12A) and CD8 (FIG. 12B) TNF-α responses elicited by mosaic Gag(WT) and N5 modified mosaic Gag (N5) polypeptides after an immunization regimen utilizing a recombinant adenoviral vector construct (rAd) containing SEQ ID NO: 16, SEQ ID NO: 17, SEQ ID NO: 18, or SEQ ID NO: 19. The bars show the positive TNF-α responses with error bars as the standard deviation.

FIG. 13 is a diagram which schematically depicts plasmid construct VRC 9656 (SEQ ID NO: 7), which comprises SEQ ID NO: 5, which is a nucleic acid sequence encoding an N5-modified mosaic Gag polypeptide.

FIG. 14 is a diagram which schematically depicts plasmid construct VRC 9657 (f NO: 8), which comprises SEQ ID NO: 3, which is a nucleic acid sequence encoding an N5-modified mosaic Gag polypeptide.

FIG. 15 is a diagram which schematically depicts plasmid construct VRC 9658 (SEQ ID NO: 9), which comprises SEQ ID NO: 4, which is a nucleic acid sequence encoding an N5-modified mosaic Gag polypeptide.

FIG. 16 is a diagram which schematically depicts plasmid construct VRC 9662 (SEQ ID NO: 10), which comprises SEQ ID NO: 6, which is a nucleic acid sequence encoding a mosaic Env polypeptide.

FIG. 17 is a diagram which schematically depicts plasmid construct VRC 9663 (SEQ ID NO: 11), which comprises SEQ ID NO: 1, which is a nucleic acid sequence encoding a mosaic Env polypeptide.

FIG. 18 is a diagram which schematically depicts plasmid construct VRC 9664 (SEQ ID NO: 12), which comprises SEQ ID NO: 2, which is a nucleic acid sequence encoding a mosaic Env polypeptide.

FIG. 19 is a diagram which schematically depicts plasmid construct VRC 4401 (SEQ ID NO: 63), which comprises SEQ ID NO: 13, which is a nucleic acid sequence encoding a wild-type clade B Gag polypeptide.

FIG. 20 is a diagram which schematically depicts plasmid construct VRC 4708 (SEQ ID NO: 64), which comprises SEQ ID NO: 14, which is a nucleic acid sequence encoding an N5-modified Gag polypeptide (non-mosaic).

FIG. 21 is a diagram which schematically depicts plasmid construct VRC 4707 (SEQ ID NO: 65), which comprises SEQ ID NO: 15, which is a nucleic acid sequence encoding an N6-modified Gag polypeptide (non-mosaic).

FIG. 22 is a diagram which schematically depicts plasmid construct VRC 4700 (SEQ ID NO: 66), which comprises SEQ ID NO: 16, which is a nucleic acid sequence encoding a wild-type mosaic Gag polypeptide.

FIG. 23 is a diagram which schematically depicts plasmid construct VRC 4704 (SEQ ID NO: 67), which comprises SEQ ID NO: 17, which is a nucleic acid sequence encoding a wild-type mosaic Gag polypeptide.

FIG. 24 is a diagram which schematically depicts plasmid construct VRC 4701 (SEQ ID NO: 68), which comprises SEQ ID NO: 18, which is a nucleic acid sequence encoding an N5-modified mosaic Gag polypeptide.

FIG. 25 is a diagram which schematically depicts plasmid construct VRC 4705 (SEQ ID NO: 69), which comprises SEQ ID NO: 19, which is a nucleic acid sequence encoding an N5-modified mosaic Gag polypeptide.

FIG. 26 is a diagram which schematically depicts plasmid construct VRC 4733 (SEQ ID NO: 49), which comprises SEQ ID NO: 35, which is a nucleic acid sequence encoding a Gag polypeptide.

FIG. 27 is a diagram which schematically depicts plasmid construct VRC 4734 (SEQ ID NO: 50), which comprises SEQ ID NO: 36, which is a nucleic acid sequence encoding a Gag polypeptide.

FIG. 28 is a diagram which schematically depicts plasmid construct VRC 4735 (SEQ ID NO: 51), which comprises SEQ ID NO: 37, which is a nucleic acid sequence encoding a Gag polypeptide.

FIG. 29 is a diagram which schematically depicts plasmid construct VRC 4736 (SEQ ID NO: 52), which comprises SEQ ID NO: 38, which is a nucleic acid sequence encoding a Gag polypeptide.

FIG. 30 is a diagram which schematically depicts plasmid construct VRC 4737 (SEQ ID NO: 53), which comprises SEQ ID NO: 39, which is a nucleic acid sequence encoding a Gag polypeptide.

FIG. 31 is a diagram which schematically depicts plasmid construct VRC 4738 (SEQ ID NO: 54), which comprises SEQ ID NO: 40, which is a nucleic acid sequence encoding a Gag polypeptide.

FIG. 32 is a diagram which schematically depicts plasmid construct VRC 4739 (SEQ ID NO: 55), which comprises SEQ ID NO: 41, which is a nucleic acid sequence encoding a Gag-Pol fusion polypeptide.

FIG. 33 is a diagram which schematically depicts plasmid construct VRC 4740 (SEQ ID NO: 56), which comprises SEQ ID NO: 42, which is a nucleic acid sequence encoding an SIV Gag polypeptide.

FIG. 34 is a diagram which schematically depicts plasmid construct VRC 4741 (SEQ ID NO: 57), which comprises SEQ ID NO: 43, which is a nucleic acid sequence encoding a Gag polypeptide.

FIG. 35 is a diagram which schematically depicts plasmid construct VRC 4742 (SEQ ID NO: 58), which comprises SEQ ID NO: 44, which is a nucleic acid sequence encoding a Gag polypeptide.

FIG. 36 is a diagram which schematically depicts plasmid construct VRC 4743 (SEQ ID NO: 59), which comprises SEQ ID NO: 45, which is a nucleic acid sequence encoding a Gag polypeptide.

FIG. 37 is a diagram which schematically depicts plasmid construct VRC 4744 (SEQ ID NO: 60), which comprises SEQ ID NO: 46, which is a nucleic acid sequence encoding a Gag polypeptide.

FIG. 38 is a diagram which schematically depicts plasmid construct VRC 4745 (SEQ ID NO: 61), which comprises SEQ ID NO: 47, which is a nucleic acid sequence encoding a Gag polypeptide.

FIG. 39 is a diagram which schematically depicts plasmid construct VRC 4746 (SEQ ID NO: 62), which comprises SEQ ID NO: 48, which is a nucleic acid sequence encoding a Gag polypeptide.

FIG. 40 is a diagram which schematically depicts plasmid construct VRC 4714 (SEQ ID NO: 71), which comprises SEQ ID NO: 70, which is a nucleic acid sequence encoding a Gag polypeptide.

FIG. 41 is a diagram which schematically depicts plasmid construct VRC 4715 (SEQ ID NO: 73), which comprises SEQ ID NO: 72, which is a nucleic acid sequence encoding a Gag polypeptide.

FIG. 42 is a diagram which schematically depicts plasmid construct VRC 4716 (SEQ ID NO: 75), which comprises SEQ ID NO: 74, which is a nucleic acid sequence encoding a Gag polypeptide.

FIG. 43 is a diagram which schematically depicts plasmid construct VRC 4717 (SEQ ID NO: 77) which comprises SEQ ID NO: 76, which is a nucleic acid sequence encoding a Gag polypeptide.

FIG. 44 is a diagram which schematically depicts plasmid construct VRC 4718 (SEQ ID NO: 79), which comprises SEQ ID NO: 78, which is a nucleic acid sequence encoding a Gag polypeptide.

FIG. 45 is a diagram which schematically depicts plasmid construct VRC 4719 (SEQ ID NO: 81), which comprises SEQ ID NO: 80, which is a nucleic acid sequence encoding a Gag polypeptide.

FIG. 46 is a diagram which schematically depicts plasmid construct VRC 4720 (SEQ ID NO: 83), which comprises SEQ ID NO: 82, which is a nucleic acid sequence encoding a Gag polypeptide.

FIG. 47 is a diagram which schematically depicts plasmid construct VRC 4721 (SEQ ID NO: 85), which comprises SEQ ID NO: 84, which is a nucleic acid sequence encoding a Gag polypeptide.

FIG. 48 is a diagram which schematically depicts plasmid construct VRC 4722 (SEQ ID NO: 87), which comprises SEQ ID NO: 86, which is a nucleic acid sequence encoding a Gag polypeptide.

FIG. 49 is a diagram which schematically depicts plasmid construct VRC 4723 (SEQ ID NO: 89), which comprises SEQ ID NO: 88, which is a nucleic acid sequence encoding a Gag polypeptide.

FIG. 50 is a diagram which schematically depicts plasmid construct VRC 4724 (SEQ ID NO: 91), which comprises SEQ ID NO: 90, which is a nucleic acid sequence encoding a Gag polypeptide.

FIG. 51 is a diagram which schematically depicts plasmid construct VRC 4725 (SEQ ID NO: 93), which comprises SEQ ID NO: 92, which is a nucleic acid sequence encoding a Gag polypeptide.

FIG. 52 is a diagram which schematically depicts plasmid construct VRC 4726 (SEQ ID NO: 95), which comprises SEQ ID NO: 94, which is a nucleic acid sequence encoding a Gag polypeptide.

FIG. 53 is a diagram which schematically depicts plasmid construct VRC 4730 (SEQ ID NO: 97), which comprises SEQ ID NO: 96, which is a nucleic acid sequence encoding a Gag polypeptide.

### DETAILED DESCRIPTION OF THE INVENTION

The invention provides an isolated or purified nucleic acid molecule comprising a nucleic acid sequence encoding an HIV Env polypeptide (i.e., gp160) which comprises or consists of SEQ ID NO: 1 or SEQ ID NO: 2. The invention provides an isolated or purified nucleic acid molecule comprising a nucleic acid sequence encoding an HIV Gag polypeptide which comprises or consists of SEQ ID NO: 3 or SEQ ID NO: 4. The invention also provides a polypeptide encoded by any of the aforementioned nucleic acid molecules. The invention further provides an isolated or purified nucleic acid molecule comprising a nucleic acid sequence that encodes the aforementioned polypeptide.

The terms “nucleic acid sequence,” “nucleic acid,” “nucleic acid molecule,” and “polynucleotide” are intended to encompass a polymer of DNA or RNA, i.e., a polynucleotide, which can be single-stranded or double-stranded and which can contain non-natural or altered nucleotides. In this respect, the terms include, as equivalents, analogs of either RNA or DNA made from nucleotide analogs and modified polynucleotides such as, though not limited to, methylated and/or capped polynucleotides.

By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term and does not mean absolute purity. It is to be understood, however, that nucleic acids and proteins may be formulated with diluents or adjuvants and nevertheless for practical purposes be isolated.

As used herein a “codon” refers to the three nucleotides which, when transcribed and translated, encode a single amino acid residue or in the case of UUA, UGA, or UAG encode a termination signal. Codons encoding amino acids are well known in the art. The inventive nucleic acid molecule preferably comprises codons used more frequently in humans than in HIV. While the genetic code is generally universal across species, the choice among synonymous codons is often species-dependent. Infrequent usage of a particular codon by an organism likely reflects a low level of the corresponding transfer RNA (tRNA) in the organism. Thus, introduction of a nucleic acid sequence into an organism which comprises codons that are not frequently utilized in the organism may result in limited expression of the nucleic acid sequence. One of ordinary skill in the art would appreciate that, to achieve an optimal immune response against HIV, the inventive nucleic acid molecule must be capable of expressing high levels of HIV polypeptide in a human host. In this respect, the inventive nucleic acid molecule preferably encodes an HIV polypeptide, but comprises codons that are more frequently expressed in mammals (e.g., humans). Such modified nucleic acid sequences are commonly described in the art as “humanized,” as “codon-optimized,” or as utilizing “mammalian-preferred” or “human-preferred” codons. Optimal codon usage is indicated by codon usage frequencies for expressed genes, as described in, for example, R. Nussinov, J. Mol. Biol., 149: 125-131 (1981).

In the context of the invention, an HIV nucleic acid sequence is said to be “codon-optimized” if at least about 60% (e.g., at least about 70%, at least about 80%, or at least about 90%) of the wild-type codons in the nucleic acid sequence are modified to encode mammalian-preferred codons. That is, an HIV nucleic acid sequence is codon-optimized if at least about 60% of the codons encoded therein are mammalian-preferred codons.

An “antigen” is a molecule that induces an immune response in a mammal. An “immune response” can entail, for example, antibody production and/or the activation of immune effector cells (e.g., T-cells). An antigen can comprise any subunit, fragment, or epitope of any proteinaceous molecule, preferably a protein or peptide of HIV-1 which ideally provokes an immune response in mammal, preferably leading to protective immunity. By “epitope” is meant a sequence on an antigen that is recognized by an antibody or an antigen receptor. Epitopes also are referred to in the art as “antigenic determinants.”

The nucleic acid sequences of SEQ ID NO: 1, SEQ ID NO: 2, SEQ ID NO: 3, and SEQ ID NO: 4 each encode an HIV Env or Gag polypeptide which comprises an insertion of at least one T-cell epitope that is not naturally present in the Gag and/or Env polypeptide. A “T-cell epitope” is an amino acid sequence of an antigen that is recognized and bound by a T-cell receptor. A “potential T-cell epitope” is an amino acid sequence of an antigen that is hypothesized to be recognized and bound by a T-cell receptor. The nucleic acid sequences SEQ ID NO: 1, SEQ ID NO: 2, SEQ ID NO: 2, or SEQ ID NO: 4 are also referred to herein as “mosaic” HIV sequences. “Mosaic” HIV sequences are generated using natural sequences as input to algorithms, such as genetic algorithms, which maximize the diversity of potential T-cell epitopes present in the natural sequences. The genetic algorithm identifies potential T-cell epitopes within the input sequences, generates potential recombinants between the input sequences, and identifies those recombinants which have the greatest diversity of T cell epitopes. Epitopes which occur infrequently may be omitted from the mosaic sequences while those which provide enhanced coverage relative to a sequence lacking that epitope may be incorporated into the mosaic sequence. Methods for generating mosaic sequences are described in, e.g., Fischer et al., Nature Medicine, 13(1): 100-106 (2007); and International Patent Application Publications WO 2007/024941 and WO 2010/042817.

The nucleic acid sequence comprising or consisting of SEQ ID NO: 1, SEQ ID NO: 2, SEQ ID NO: 3, or SEQ ID NO: 4 can be modified in any suitable manner for any purpose, such as, for example, to enhance the immunogenicity of the Env or Gag polypeptide encoded thereby, or to enhance the expression of the nucleic acid sequence in vivo. In this respect, the nucleic acid sequence comprising or consisting of SEQ ID NO: 1, SEQ ID NO: 2, SEQ ID NO: 3, or SEQ ID NO: 4 can be mutated to produce a modified HIV Env or Gag polypeptide using any suitable method known in the art. Such methods include, for example, insertion, deletion, and/or modification of one or more nucleotides. For example, mutations may be introduced into a SEQ ID NO: 1, SEQ ID NO: 2, SEQ ID NO: 3, or SEQ ID NO: 4 randomly (e.g., by error-prone PCR) or in a site-specific manner (see, e.g., Walder et al., Gene, 42: 133 (1986); and U.S. Pat. Nos. 4,518,584 and 4,737,462)). In addition, the nucleic acid sequence encoding an Env polypeptide (i.e., SEQ ID NO: 1 or SEQ ID NO: 2) can comprise mutations in the cleavage site, fusion peptide, or interhelical coiled-coil domains of a wild-type Env protein (ΔCFI Env proteins), which expose the core protein for optimal antigen presentation and recognition (see, e.g., U.S. Pat. No. 7,470,430; Cao et al., J. Virol., 71: 9808-9812 (1997); Yang et al., J. Virol., 78: 4029-4036 (2004)). In addition, the Env polypeptide can lack the cytoplasmic domain of a wild-type Env protein. The Env polypeptide also can lack one or more variable loops of a wild-type Env polypeptide. For example, the inventive nucleic acid molecule preferably does not encode the variable loops 1, 2, 3, 4, or 5 of Env, or combinations thereof (see, e.g., International Patent Application Publication WO 2005/034992). Mutant Gag polypeptides are disclosed in, e.g., U.S. Pat. No. 7,608,422, and Shimano et al., Virus Genes, 18(3): 197-220 (1999).

In some embodiments, the nucleic acid molecule comprising or consisting of the nucleic acid sequence of SEQ ID NO: 1, SEQ ID NO: 2, SEQ ID NO: 3, or SEQ ID NO: 4 encodes one or more additional HIV polypeptides or antigens (e.g., 2, 3, 4, 5, 10, or more polypeptides or antigens). Examples of other suitable HIV polypeptides include, but are not limited to, all or part of an HIV Pol, Tat, Reverse Transcriptase (RT), Vif, Vpr, Vpu, Vpo, Integrase, and Nef proteins. The additional HIV polypeptide or antigen can be from any group or clade of HIV. HIV-1 can be classified into four groups: the “major” group M, the “outlier” group O, group N, and group P. Preferably, the nucleic acid sequence comprising or consisting of SEQ ID NO: 1, SEQ ID NO: 2, SEQ ID NO: 3, or SEQ ID NO: 4 further encodes an HIV polypeptide from group M. Within group M, there are several genetically distinct clades (or subtypes) of HIV-1. Thus, the nucleic acid molecule comprising or consisting of SEQ ID NO: 1, SEQ ID NO: 2, SEQ ID NO: 3, or SEQ ID NO: 4 can further encode an HIV polypeptide from HIV-1 clade A, B, C, D, E, F, G, H, J, or K, or the like. In one embodiment, the inventive nucleic acid molecule can comprise an additional nucleic acid sequence which encodes an HIV Gag or Env polypeptide that is derived from an HIV clade that is different from the HIV clade of SEQ ID NO: 1, SEQ ID NO: 2, SEQ ID NO: 3, or SEQ ID NO: 4. Alternatively, the inventive nucleic acid molecule can comprise one or more additional nucleic acid sequences (e.g., 2, 3, 4, 5, 10, or more nucleic acid sequences) which encode an HIV polypeptide from the same clade as the polypeptide encoded by SEQ ID NO: 1, SEQ ID NO: 2, SEQ ID NO: 3, or SEQ ID NO: 4. HIV Gag, Env, and Pol proteins from the different HIV clades, as well as nucleic acid sequences encoding such proteins and methods for the manipulation and insertion of such nucleic acid sequences into vectors, are known (see, e.g., HIV Sequence Compendium, Division of AIDS, National Institute of Allergy and Infectious Diseases (2003); HIV Sequence Database (hiv-web.lanl.gov/content/hiv-db/mainpage.html); Sambrook et al., Molecular Cloning, a Laboratory Manual, 2d edition, Cold Spring Harbor Press, Cold Spring Harbor, N.Y. (1989); Ausubel et al., Current Protocols in Molecular Biology, Greene Publishing Associates and John Wiley & Sons, New York, N.Y. (1994)).

In embodiments where the inventive nucleic acid molecule encodes one or more additional HIV polypeptides, the inventive nucleic acid molecule can comprise one or more additional nucleic acid sequences (e.g., 2, 3, 4, 5, 10, or more nucleic acid sequences) which encode fragments (e.g., epitopes or other antigenic fragments) of an HIV protein, such as any of the HIV proteins described herein. Antigenic fragments and epitopes of the HIV Gag, Env, and Pol proteins, as well as nucleic acid sequences encoding such antigenic fragments and epitopes, are known (see, e.g., HIV Immunology and HIV/SIV Vaccine Databases, Vol. 1, Division of AIDS, National Institute of Allergy and Infectious Diseases (2003)). Alternatively, the inventive nucleic acid molecule sequence can comprise one or more additional nucleic acid sequences (e.g., 2, 3, 4, 5, 10, or more nucleic acid sequences) that encode a fusion protein or polypeptide. The fusion protein can comprise all or part of any of the HIV polypeptides described herein. For example, all or part of an HIV Env protein (e.g., gp120 or gp160) can be fused to all or part of the HIV Pol protein, or all or part of HIV Gag protein can be fused to all or part of the HIV Pol protein. Such fusion proteins effectively provide multiple HIV antigens in the context of the invention, and can be used to generate a more complete immune response against a given HIV pathogen as compared to that generated by a single HIV antigen.

In another embodiment, the inventive nucleic acid molecule, which comprises or consists of a nucleic acid sequence of SEQ ID NO: 1, SEQ ID NO: 2, SEQ ID NO: 3, or SEQ ID NO: 4 can comprise one or more additional nucleic acid sequences (e.g., 2, 3, 4, 5, 10, or more nucleic acid sequences) that encode antigens derived from other mammalian species, such as non-human primates. In this respect, the inventive nucleic acid molecule can further comprise a nucleic acid sequence derived from a simian immunodeficiency virus (SIV) that encodes one or more T-cell epitopes which are not naturally found in the HIV polypeptide. The immunogenicity of HIV-1 is much lower than the immunogenicity of SIV. Therefore, such chimeric HIV/SIV polypeptides can increase the breadth and potency of the T-cell response directed against HIV.

The invention also provides a vector comprising the nucleic acid molecule described herein. A “vector” is a molecule, such as plasmid, phage, cosmid, liposome, molecular conjugate (e.g., transferrin), or virus, into which another nucleic acid sequence may be introduced so as to bring about the replication of the inserted sequence. Preferably, the vector is a plasmid or a viral vector. The term “construct,” as used herein, refers to a vector (e.g., a plasmid or adenoviral vector) containing a nucleic acid sequence inserted therein. Thus, the invention also provides a construct comprising a vector (e.g., a plasmid vector or a viral vector) having inserted therein a nucleic acid molecule comprising or consisting of a nucleic acid sequence of SEQ ID NO: 1, SEQ ID NO: 2, SEQ ID NO: 3, or SEQ ID NO: 4. Such constructs are referred to herein as “plasmid constructs,” “plasmid vector constructs,” or “viral vector constructs” (e.g., adenoviral vector constructs). An “empty” or “null” vector is a vector that does not contain a heterologous nucleic acid sequence inserted therein. In one embodiment, SEQ ID NOs: 8 and 9 correspond to plasmid constructs which contain the Gag-encoding nucleic acid sequence inserts of SEQ ID NO: 3 and SEQ ID NO: 4, respectively. SEQ ID NOs: 11 and 12 correspond to plasmid constructs which contain the Env-encoding nucleic acid sequence inserts of SEQ ID NO: 1 and SEQ ID NO: 2, respectively.

Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., supra, and Ausubel et al., supra.

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. As such, long-term expression of a therapeutic factor(s) is achievable when using retrovirus. Retroviruses contemplated for use in human gene transfer are relatively non-pathogenic, although pathogenic retroviruses exist. When employing pathogenic retroviruses, e.g., human immunodeficiency virus (HIV) or human T-cell lymphotrophic viruses (HTLV), care must be taken in altering the viral genome to eliminate toxicity to the host. A retroviral vector additionally can be manipulated to render the virus replication-deficient. As such, retroviral vectors are considered particularly useful for stable gene transfer in vivo.

An HSV-based viral vector is suitable for use as a vector to introduce a nucleic acid into numerous cell types. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, 5,849,572, and 5,804,413, and International Patent Application Publications WO 91/02788, WO 96/04394, WO 98/15637, and WO 99/06583.

AAV vectors are viral vectors of particular interest for use in human gene transfer. AAV is a DNA virus, which is not known to cause human disease. The AAV genome is comprised of two genes, rep and cap, flanked by inverted terminal repeats (ITRs), which contain recognition signals for DNA replication and packaging of the virus. AAV requires co-infection with a helper virus (i.e., an adenovirus or a herpes simplex virus), or expression of helper genes, for efficient replication. AAV can be propagated in a wide array of host cells including human, simian, and rodent-cells, depending on the helper virus employed. An AAV vector used for administration of a nucleic acid sequence typically has approximately 96% of the parental genome deleted, such that only the ITRs remain. This eliminates immunologic or toxic side effects due to expression of viral genes. If desired, the AAV rep protein can be co-administered with the AAV vector to enable integration of the AAV vector into the host cell genome. Host cells comprising an integrated AAV genome show no change in cell growth or morphology (see, e.g., U.S. Pat. No. 4,797,368). As such, prolonged expression of therapeutic factors from AAV vectors can be useful in treating persistent and chronic diseases.

In one embodiment, the vector is a recombinant Lymphocytic Choriomeningitis Virus (LCMV) vector. Recombinant LCMV is used in the art to study both acute and persistent viral infection, virus-host balance, and associated disease. LCMV is an enveloped bisegmented negative-strand RNA virus. The two genome segments L and S have approximate sizes of 7.2 and 3.4 kb, respectively. Each segment uses an ambisense strategy to direct the synthesis of two proteins in opposite orientations, separated by an intergenic region. The S RNA contains the nucleoprotein (NP) and the glycoprotein (GP) precursor (GPC) genes, which are encoded in antigenome and genome polarity, respectively. Posttranslational processing of GPC genes produces GP-1 and -2 and has been shown to be mediated by the cellular protease S1P. GP-1 and -2 make up the spikes on the virion envelope and mediate cell entry by interaction with the host cell surface receptor. The L RNA segment codes for the virus RNA-dependent RNA polymerase (L) and a small (11-kDa) RING finger protein (Z) (see, e.g., Pinschewer et al., Proc. Natl. Acad. Sci., 100(13): 7895-7900 (2003)). Recombinant LCMV vectors are described in, for example, Pinschewer et al., supra, and Flatz et al., Nature Medicine, 16: 339-345 (2010).

In a preferred embodiment, the vector is an adenoviral vector. Adenoviruses are generally associated with benign pathologies in humans, and the 36 kilobase (kb) adenoviral genome has been extensively studied. Adenoviral vectors can be produced in high titers (e.g., about 10¹³ particle forming units (pfu)), and can transfer genetic material to nonreplicating, as well as replicating, cells; in contrast with, e.g., retroviral vectors, which only transfer genetic material to replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3, 147-154 (1992)). Additionally, adenoviruses generally do not integrate into the host cell chromosome, but rather are maintained as a linear episome, thus minimizing the likelihood that a recombinant adenovirus will interfere with normal cell function. In addition to being a superior vehicle for transferring genetic material to a wide variety of cell types, adenoviral vectors represent a safe choice for gene transfer, a particular concern for therapeutic applications.

Adenovirus from various origins, subtypes, or mixture of subtypes can be used as the source of the viral genome for the adenoviral vector. Non-human adenovirus (e.g., simian, chimpanzee, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, or SAV. Preferably, the simian adenoviral vector is a simian adenoviral vector of serotype 3, 7, 11, 16, 18, 19, 20, 27, 33, 38, or 39. More preferably, the simian adenoviral vector is of serotype 7, 11, 16, 18, or 38.

Human adenovirus preferably is used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Preferably, the adenoviral vector is of human subgroup C, especially serotype 2 or even more desirably serotype 5. However, non-group C adenoviruses can be used to prepare adenoviral vectors for delivery of gene products to host cells. Preferred adenoviruses used in the construction of non-group C adenoviral gene transfer vectors include Ad35 (group B), Ad26 (group D), and Ad28 (group D). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561 and International Patent Application Publications WO 97/12986 and WO 98/53087.

The adenoviral vector can be replication-competent. For example, the adenoviral vector can have a mutation (e.g., a deletion, an insertion, or a substitution) in the adenoviral genome that does not inhibit viral replication in host cells. The adenoviral vector also can be conditionally replication-competent. Preferably, however, the adenoviral vector is replication-deficient in host cells.

By “replication-deficient” is meant that the adenoviral vector requires complementation of one or more regions of the adenoviral genome that are required for replication, as a result of, for example a deficiency in at least one replication-essential gene function (i.e., such that the adenoviral vector does not replicate in typical host cells, especially those in a human patient that could be infected by the adenoviral vector in the course of the inventive method). A deficiency in a gene, gene function, or genomic region, as used herein, is defined as a deletion of sufficient genetic material of the viral genome to obliterate or impair the function of the gene (e.g., such that the function of the gene product is reduced by at least about 2-fold, 5-fold, 10-fold, 20-fold, 30-fold, or 50-fold) whose nucleic acid sequence was deleted in whole or in part. Deletion of an entire gene region often is not required for disruption of a replication-essential gene function. However, for the purpose of providing sufficient space in the adenoviral genome for one or more transgenes, removal of a majority of a gene region may be desirable. While deletion of genetic material is preferred, mutation of genetic material by addition or substitution also is appropriate for disrupting gene function. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA1 and/or VA-RNA-2).

The replication-deficient adenoviral vector desirably requires complementation of at least one replication-essential gene function of one or more regions of the adenoviral genome. Preferably, the adenoviral vector requires complementation of at least one gene function of the E1A region, the E1B region, or the E4 region of the adenoviral genome required for viral replication (denoted an E1-deficient or E4-deficient adenoviral vector). In addition to a deficiency in the E1 region, the recombinant adenovirus also can have a mutation in the major late promoter (MLP), as discussed in International Patent Application Publication WO 00/00628. Most preferably, the adenoviral vector is deficient in at least one replication-essential gene function (desirably all replication-essential gene functions) of the E1 region and at least one gene function of the nonessential E3 region (e.g., an Xba I deletion of the E3 region) (denoted an E1/E3-deficient adenoviral vector). With respect to the E1 region, the adenoviral vector can be deficient in part or all of the E1A region and/or part or all of the E1B region, e.g., in at least one replication-essential gene function of each of the E1A and E1B regions, thus requiring complementation of the E1A region and the E1B region of the adenoviral genome for replication. The adenoviral vector also can require complementation of the E4 region of the adenoviral genome for replication, such as through a deficiency in one or more replication-essential gene functions of the E4 region.

When the adenoviral vector is deficient in at least one replication-essential gene function in one region of the adenoviral genome (e.g., an E1- or E1/E3-deficient adenoviral vector), the adenoviral vector is referred to as “singly replication-deficient.” A particularly preferred singly replication-deficient adenoviral vector is, for example, a replication-deficient adenoviral vector requiring, at most, complementation of the E1 region of the adenoviral genome, so as to propagate the adenoviral vector (e.g., to form adenoviral vector particles).

The adenoviral vector of the invention can be “multiply replication-deficient,” meaning that the adenoviral vector is deficient in one or more replication-essential gene functions in each of two or more regions of the adenoviral genome, and requires complementation of those functions for replication. For example, the aforementioned E1-deficient or E1/E3-deficient adenoviral vector can be further deficient in at least one replication-essential gene function of the E4 region (denoted an E1/E4- or E1/E3/E4-deficient adenoviral vector), and/or the E2 region (denoted an E1/E2- or E1/E2/E3-deficient adenoviral vector), preferably the E2A region (denoted an E1/E2A- or E1/E2A/E3-deficient adenoviral vector). An adenoviral vector deleted of the entire E4 region can elicit a lower host immune response.

In one embodiment of the invention, the adenoviral vector can comprise an adenoviral genome deficient in one or more replication-essential gene functions of each of the E1 and E4 regions (i.e., the adenoviral vector is an E1/E4-deficient adenoviral vector), preferably with the entire coding region of the E4 region having been deleted from the adenoviral genome. In other words, all the open reading frames (ORFs) of the E4 region have been removed. Most preferably, the adenoviral vector is rendered replication-deficient by deletion of all of the E1 region and by deletion of a portion of the E4 region. The E4 region of the adenoviral vector can retain the native E4 promoter, polyadenylation sequence, and/or the right-side inverted terminal repeat (ITR).

The adenoviral vector, when multiply replication-deficient, especially in replication-essential gene functions of the E1 and E4 regions, can include a spacer sequence to provide viral growth in a complementing cell line similar to that achieved by singly replication-deficient adenoviral vectors, particularly an E1-deficient adenoviral vector. The spacer sequence can contain any nucleotide sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer sequence can be coding or non-coding and native or non-native with respect to the adenoviral genome, but does not restore the replication-essential function to the deficient region. The spacer can also contain a promoter-variable expression cassette. More preferably, the spacer comprises an additional polyadenylation sequence and/or a passenger gene. Preferably, in the case of a spacer inserted into a region deficient for E4, both the E4 polyadenylation sequence and the E4 promoter of the adenoviral genome or any other (cellular or viral) promoter remain in the vector. The spacer is located between the E4 polyadenylation site and the E4 promoter, or, if the E4 promoter is not present in the vector, the spacer is proximal to the right-side ITR. The spacer can comprise any suitable polyadenylation sequence. Examples of suitable polyadenylation sequences include synthetic optimized sequences, BGH (Bovine Growth Hormone), Polyoma virus, TK (Thymidine Kinase), EBV (Epstein Barr Virus), and the papillomaviruses, including human papillomaviruses and BPV (Bovine Papilloma Virus). Preferably, particularly in the E4 deficient region, the spacer includes an SV40 Polyadenylation sequence. The SV40 polyadenylation sequence allows for higher virus production levels of multiply replication deficient adenoviral vectors. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. Ideally, the spacer comprises the glucuronidase gene. The use of a spacer in an adenoviral vector is further described in, for example, U.S. Pat. No. 5,851,806 and International Patent Application Publication WO 97/21826.

Desirably, the adenoviral vector requires, at most, complementation of replication-essential gene functions of the E1, E2A, and/or E4 regions of the adenoviral genome for replication (i.e., propagation). However, the adenoviral genome can be modified to disrupt one or more replication-essential gene functions as desired by the practitioner, so long as the adenoviral vector remains deficient and can be propagated using, for example, complementing cells and/or exogenous DNA (e.g., helper adenovirus) encoding the disrupted replication-essential gene functions. In this respect, the adenoviral vector can be deficient in replication-essential gene functions of only the early regions of the adenoviral genome, only the late regions of the adenoviral genome, both the early and late regions of the adenoviral genome, or all adenoviral genes (i.e., a high capacity adenovector (HC-Ad); see Morsy et al., Proc. Natl. Acad. Sci. USA, 95: 965-976 (1998); Chen et al., Proc. Natl. Acad. Sci USA, 94: 1645-1650 (1997); Kochanek et al., Hum. Gene Ther., 10: 2451-2459 (1999)). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Applications WO 94/28152, WO 95/02697, WO 95/16772, WO 95/34671, WO 96/22378, WO 97/12986, WO 97/21826, and WO 03/022311.

By removing all or part of, for example, the E1, E3, and E4 regions of the adenoviral genome, the resulting adenoviral vector is able to accept inserts of exogenous nucleic acid sequences while retaining the ability to be packaged into adenoviral capsids (thereby resulting in adenoviral vector constructs). The inventive nucleic acid molecule can be positioned in the E1 region, the E3 region, or the E4 region of the adenoviral genome. Indeed, the nucleic acid molecule can be inserted anywhere in the adenoviral genome so long as the position does not prevent expression of the nucleic acid sequence or interfere with packaging of the adenoviral vector. In addition to the inventive nucleic acid molecule comprising or consisting of a nucleic acid sequence of SEQ ID NO: 1, SEQ ID NO: 2, SEQ ID NO: 3, or SEQ ID NO: 4, the adenoviral vector also can comprise one or more (i.e., two or more) additional nucleic acid sequences encoding the same or different HIV polypeptide. Each nucleic acid sequence can be operably linked to the same promoter, or to different promoters depending on the expression profile desired by the practitioner, and can be inserted in the same region of the adenoviral genome (e.g., the E4 region) or in different regions of the adenoviral genome (e.g., one nucleic acid sequence is inserted into the E1 region, and a second nucleic acid sequence is inserted into the E4 region).

In one embodiment, the adenoviral vector can comprise any one or combination of SEQ ID NO: 1, SEQ ID NO: 2, SEQ ID NO: 3, and/or SEQ ID NO: 4. For example, the adenoviral vector can comprise (a) SEQ ID NO: 1 and SEQ ID NO: 2, (b) SEQ ID NO: 1 and SEQ ID NO: 3, (c) SEQ ID NO: 1 and SEQ ID NO: 4, (d) SEQ ID NO: 2 and SEQ ID NO: 3, (e) SEQ ID NO: 2 and SEQ ID NO: 4, (f) SEQ ID NO: 3 and SEQ ID NO: 4, (g) SEQ ID NO: 1, SEQ ID NO: 2, and SEQ ID NO: 3, (h) SEQ ID NO: 2, SEQ ID NO: 3, and SEQ ID NO: 4, (i) SEQ ID NO: 1, SEQ ID NO: 3, and SEQ ID NO: 4, (j) SEQ ID NO: 1, SEQ ID NO: 2, and SEQ ID NO: 4, or (k) SEQ ID NO: 1, SEQ ID NO: 2, SEQ ID NO: 3, and SEQ ID NO: 4.

In another embodiment, the adenoviral vector can comprise the inventive nucleic acid molecule, and one or more additional nucleic acid sequences that each encode a different HIV antigen. For example, the adenoviral vector can comprise SEQ ID NO: 1, SEQ ID NO: 2, SEQ ID NO: 3, or SEQ ID NO: 4 and multiple other nucleic acid sequences, each of which encodes a modified HIV polypeptide which comprises an insertion of at least one T-cell epitope that is not naturally present in the polypeptide. In this respect, the multiple other nucleic acid sequences in the adenoviral vector can encode (a) a modified Gag polypeptide and a modified Env polypeptide, (b) a modified Gag polypeptide and a modified Pol polypeptide, (c) a modified Env polypeptide and a modified Pol polypeptide, (d) a modified Env polypeptide and a modified Nef polypeptide, (e) a modified Gag polypeptide and a modified Nef polypeptide, (f) a modified Pol polypeptide and a modified Nef polypeptide, (g) a modified Gag polypeptide, a modified Pol polypeptide, and a modified Env polypeptide, (h) a modified Gag polypeptide, a modified Pol polypeptide, and a modified Nef polypeptide, (i) a modified Pol polypeptide, a modified Env polypeptide, and a modified Nef polypeptide, or (j) a modified Gag polypeptide, a modified Env polypeptide, and a modified Nef polypeptide.

Replication-deficient adenoviral vectors are typically produced in complementing cell lines that provide gene functions not present in the replication-deficient adenoviral vectors, but required for viral propagation, at appropriate levels in order to generate high titers of viral vector stock. Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application Publication WO 97/00326, and U.S. Pat. Nos. 5,994,128 and 6,033,908), and 293-ORF6 cells (described in, e.g., International Patent Application Publication WO 95/34671 and Brough et al., J. Virol., 71: 9206-9213 (1997)). Additional complementing cells are described in, for example, U.S. Pat. Nos. 6,677,156 and 6,682,929, and International Patent Application Publication WO 03/20879. In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the desired adenoviral vector.

If the adenoviral vector is not replication-deficient, ideally the adenoviral vector is manipulated to limit replication of the vector to within a target tissue. The adenoviral vector can be a conditionally-replicating adenoviral vector, which is engineered to replicate under conditions pre-determined by the practitioner. For example, replication-essential gene functions, e.g., gene functions encoded by the adenoviral early regions, can be operably linked to an inducible, repressible, or tissue-specific transcription control sequence, e.g., promoter. In this embodiment, replication requires the presence or absence of specific factors that interact with the transcription control sequence. Conditionally-replicating adenoviral vectors are described further in U.S. Pat. No. 5,998,205.

The coat protein of an adenoviral vector can be manipulated to alter the binding specificity or recognition of the virus for a viral receptor on a potential host cell. For adenovirus, such manipulations can include deletion of regions of the fiber, penton, or hexon, insertions of various native or non-native ligands into portions of the coat protein, and the like. Manipulation of the coat protein can broaden the range of cells infected by the adenoviral vector or enable targeting of the adenoviral vector to a specific cell type.

Any suitable technique for altering native binding to a host cell, such as native binding of the fiber protein to the coxsackievirus and adenovirus receptor (CAR) of a cell, can be employed (see, e.g., U.S. Patent Application Publication 2009/0148477, and U.S. Pat. No. 5,962,311). In addition, the nucleic acid residues encoding amino acid residues associated with native substrate binding can be changed, supplemented, or deleted (see, e.g., International Patent Application Publication WO 00/15823; Einfeld et al., J. Virol., 75(23): 11284-11291 (2001); van Beusechem et al., J. Virol., 76(6): 2753-2762 (2002)) such that the adenoviral vector incorporating the mutated nucleic acid residues (or having the fiber protein encoded thereby) is less able to bind its native substrate. In this respect, the native CAR and integrin binding sites of the adenoviral vector, such as the knob domain of the adenoviral fiber protein and an Arg-Gly-Asp (RGD) sequence located in the adenoviral penton base, respectively, can be removed or disrupted. In one embodiment, the adenoviral vector comprises a fiber protein and a penton base protein that do not bind to CAR and integrins, respectively. Alternatively, the adenoviral vector comprises fiber protein and a penton base protein that bind to CAR and integrins, respectively, but with less affinity than the corresponding wild-type coat proteins. The adenoviral vector exhibits reduced binding to CAR and integrins if a modified adenoviral fiber protein and penton base protein binds CAR and integrins, respectively, with at least about 5-fold, 10-fold, 20-fold, 30-fold, 50-fold, or 100-fold less affinity than a non-modified adenoviral fiber protein and penton base protein of the same serotype.

The adenoviral vector also can comprise a chimeric coat protein comprising a non-native amino acid sequence that binds a substrate (i.e., a ligand), such as a cellular receptor other than CAR the αv integrin receptor. Such a chimeric coat protein allows an adenoviral vector to bind, and desirably, infect host cells not naturally infected by the corresponding adenovirus that retains the ability to bind native cell surface receptors, thereby further expanding the repertoire of cell types infected by the adenoviral vector. A “non-native” amino acid sequence can comprise an amino acid sequence not naturally present in the adenoviral coat protein or an amino acid sequence found in the adenoviral coat but located in a non-native position within the capsid. By “preferentially binds” is meant that the non-native amino acid sequence binds a receptor, such as, for instance, αvβ3 integrin, with at least about 3-fold greater affinity (e.g., at least about 5-fold, 10-fold, 15-fold, 20-fold, 25-fold, 35-fold, 45-fold, or 50-fold greater affinity) than the non-native ligand binds a different receptor, such as, for instance, αvβ1 integrin.

Desirably, the adenoviral vector comprises a chimeric coat protein comprising a non-native amino acid sequence that confers to the chimeric coat protein the ability to bind to an immune cell more efficiently than a wild-type adenoviral coat protein. In particular, the adenoviral vector can comprise a chimeric adenoviral fiber protein comprising a non-native amino acid sequence which facilitates uptake of the adenoviral vector by immune cells, preferably antigen presenting cells, such as dendritic cells, monocytes, and macrophages. In a preferred embodiment, the adenoviral vector comprises a chimeric fiber protein comprising an amino acid sequence (e.g., a non-native amino acid sequence) comprising an RGD motif, which increases transduction efficiency of an adenoviral vector into dendritic cells. The RGD-motif, or any non-native amino acid sequence, preferably is inserted into the adenoviral fiber knob region, ideally in an exposed loop of the adenoviral knob, such as the HI loop. A non-native amino acid sequence also can be appended to the C-terminus of the adenoviral fiber protein, optionally via a spacer sequence. The spacer sequence preferably comprises between one and two-hundred amino acids, and can (but need not) have an intended function.

In another embodiment, the adenoviral vector can comprise a chimeric virus coat protein that is not selective for a specific type of eukaryotic cell. The chimeric coat protein differs from a wild-type coat protein by an insertion of a non-native amino acid sequence into or in place of an internal coat protein sequence, or attachment of a non-native amino acid sequence to the N- or C-terminus of the coat protein (see, e.g., U.S. Pat. No. 6,465,253 and International Patent Application Publication WO 97/20051).

A non-native amino acid sequence can be conjugated to any of the adenoviral coat proteins to form a chimeric adenoviral coat protein. Therefore, for example, a non-native amino acid sequence can be conjugated to, inserted into, or attached to a fiber protein, a penton base protein, a hexon protein, proteins IX, VI, or IIIa, etc. The sequences of such proteins, and methods for employing them in recombinant proteins, are well known in the art (see, e.g., U.S. Pat. Nos. 5,543,328; 5,559,099; 5,712,136; 5,731,190; 5,756,086; 5,770,442; 5,846,782; 5,962,311; 5,965,541; 5,846,782; 6,057,155; 6,127,525; 6,153,435; 6,329,190; 6,455,314; 6,465,253; 6,576,456; 6,649,407; 6,740,525; and 6,951,755, and International Patent Application Publications WO 96/07734, WO 96/26281, WO 97/20051, WO 98/07877, WO 98/07865, WO 98/40509, WO 98/54346, WO 00/15823, WO 01/58940, and WO 01/92549). The chimeric adenoviral coat protein can be generated using standard recombinant DNA techniques known in the art. Preferably, the nucleic acid sequence encoding the chimeric adenoviral coat protein is located within the adenoviral genome and is operably linked to a promoter that regulates expression of the coat protein in a wild-type adenovirus. Alternatively, the nucleic acid sequence encoding the chimeric adenoviral coat protein is located within the adenoviral genome and is part of an expression cassette which comprises genetic elements required for efficient expression of the chimeric coat protein.

Disruption of native binding of adenoviral coat proteins to a cell surface receptor can also render it less able to interact with the innate or acquired host immune system. Aside from pre-existing immunity, adenoviral vector administration induces inflammation and activates both innate and acquired immune mechanisms. Adenoviral vectors activate antigen-specific (e.g., T-cell dependent) immune responses, which limit the duration of transgene expression following an initial administration of the vector. In addition, exposure to adenoviral vectors stimulates production of neutralizing antibodies by B cells, which can preclude gene expression from subsequent doses of adenoviral vector (Wilson & Kay, Nat. Med., 3(9): 887-889 (1995)). Indeed, the effectiveness of repeated administration of the vector can be severely limited by host immunity. In addition to stimulation of humoral immunity, cell-mediated immune functions are responsible for clearance of the virus from the body. Rapid clearance of the virus is attributed to innate immune mechanisms (see, e.g., Worgall et al., Human Gene Therapy, 8: 37-44 (1997)), and likely involves Kupffer cells found within the liver. Thus, by ablating native binding of an adenovirus fiber protein and penton base protein, immune system recognition of an adenoviral vector is diminished, thereby increasing vector tolerance by the host.

Another method for evading pre-existing host immunity to adenovirus, especially serotype 5 adenovirus, involves modifying an adenoviral coat protein such that it exhibits reduced recognition by the host immune system. Thus, the adenoviral vector preferably comprises such a modified coat protein. The modified coat protein preferably is a penton, fiber, or hexon protein. Most preferably, the modified coat protein is a hexon protein. The coat protein can be modified in any suitable manner, but is preferably modified by generating diversity in the coat protein. Preferably, such coat protein variants are not recognized by pre-existing host (e.g., human) adenovirus-specific neutralizing antibodies. Diversity can be generated using any suitable method known in the art, including, for example, directed evolution (i.e., polynucleotide shuffling) and error-prone PCR (see, e.g., Cadwell, PCR Meth. Appl., 2: 28-33 (1991); Leung et al., Technique, 1: 11-15 (1989); Pritchard et al., J. Theoretical Biol., 234: 497-509 (2005)). Preferably, coat protein diversity is generated through directed evolution techniques, such as those described in, e.g., Stemmer, Nature, 370: 389-91 (1994); Chemy et al., Nat. Biotechnol., 17: 379-84 (1999); Schmidt-Dannert et al., Nat Biotechnol., 18(7): 750-53 (2000); U.S. Patent Application Publication 2009/0148477.

An adenoviral coat protein also can be modified to evade pre-existing host immunity by deleting a region of a coat protein and replacing it with a corresponding region from the coat protein of another adenovirus serotype, particularly a serotype which is less immunogenic in humans. In this regard, amino acid sequences within the fiber protein, the penton base protein, and/or the hexon protein can be removed and replaced with corresponding sequences from a different adenovirus serotype. Thus, for example, when the fiber protein is modified to evade pre-existing host immunity, amino acid residues from the knob region of a serotype 5 fiber protein can be deleted and replaced with corresponding amino acid residues from an adenovirus of a different serotype, such as those serotypes described herein. Likewise, when the penton base protein is modified to evade pre-existing host immunity, amino acid residues within the hypervariable region of a serotype 5 penton base protein can be deleted and replaced with corresponding amino acid residues from an adenovirus of a different serotype, such as those serotypes described herein. Preferably, the hexon protein of the adenoviral vector is modified in this manner to evade pre-existing host immunity. In this respect, when the adenoviral vector is of serotype 5, amino acid residues within one or more of the hypervariable regions, which occur in loops of the hexon protein, are removed and replaced with corresponding amino acid residues from an adenovirus of a different serotype. Preferably, amino acid residues within the FG1, FG2, or DE1 loops of a serotype 5 hexon protein are deleted and replaced with corresponding amino acid residues from a hexon protein of a different adenovirus serotype. An entire loop region can be removed from the serotype 5 hexon protein and replaced with the corresponding loop region of another adenovirus serotype. Alternatively, portions of a loop region can be removed from the serotype 5 hexon protein and replaced with the corresponding portion of a hexon loop of another adenovirus serotype. One or more hexon loops, or portions thereof, of a serotype 5 adenoviral vector can be removed and replaced with the corresponding sequences from any other adenovirus serotype, such as those described herein. The structure of Ad2 and Ad5 hexon proteins and methods of modifying hexon proteins are disclosed in, for example, Rux et al., J. Virol., 77: 9553-9566 (2003), and U.S. Pat. No. 6,127,525. The hypervariable regions of a hexon protein also can be replaced with random peptide sequences, or peptide sequences derived from a disease-causing pathogen (e.g., HIV).

Modifications to adenovirus coat proteins are described in, for example, U.S. Pat. Nos. 5,543,328; 5,559,099; 5,712,136; 5,731,190; 5,756,086; 5,770,442; 5,846,782; 5,871,727; 5,885,808; 5,922,315; 5,962,311; 5,965,541; 6,057,155; 6,127,525; 6,153,435; 6,329,190; 6,455,314; 6,465,253; 6,576,456; 6,649,407; 6,740,525; and 6,951,755; and International Patent Applications WO 96/07734, WO 96/26281, WO 97/20051, WO 98/07865, WO 98/07877, WO 98/40509, WO 98/54346, WO 00/15823, WO 01/58940, and WO 01/92549.

The constructs (e.g., plasmid constructs or adenoviral vector constructs) of the invention comprise a nucleic acid sequence encoding any of the HIV polypeptides described herein in a form suitable for expression of the nucleic acid sequence in a host cell, which means that the constructs include one or more sequences which regulate expression of the nucleic acid sequence. Such regulatory sequences are operatively linked to the nucleic acid sequence to be expressed. By “operably linked” is meant that the nucleic acid sequence is linked to the regulatory sequence(s) in a manner which allows for expression of the nucleic acid sequence. The term “regulatory sequence” is intended to include promoters, enhancers, and other expression control elements (e.g., polyadenylation signals). Such regulatory sequences are described, for example, in Goeddel, Gene Expression Technology: Methods in Enzymology, 185, Academic Press, San Diego, Calif. (1990).

Any promoter or enhancer sequence can be used in the context of the invention, so long as sufficient expression of the inventive nucleic acid sequence is achieved and a robust immune response against the encoded polypeptide is generated. In this regard, the promoter can be a viral promoter. Suitable viral promoters include, for example, cytomegalovirus (CMV) promoters, such as the mouse CMV immediate-early promoter (mCMV) or the human CMV immediate-early promoter (hCMV) (described in, for example, U.S. Pat. Nos. 5,168,062 and 5,385,839), Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78: 144-145 (1981)), promoters derived from SV40 or Epstein Barr virus, an adeno-associated viral promoter, such as the p5 promoter, and the like. Preferably, the promoter is the CMV immediate-early promoter (mouse or human).

Alternatively, the promoter can be a cellular promoter, i.e., a promoter that is native to eukaryotic, preferably animal, cells. In one aspect, the cellular promoter is preferably a constitutive promoter that works in a variety of cell types, such as cells associated with the immune system. Suitable constitutive promoters can drive expression of genes encoding transcription factors, housekeeping genes, or structural genes common to eukaryotic cells.

Suitable cellular promoters include, for example, a ubiquitin promoter (e.g., a UbC promoter) (see, e.g., Marinovic et al., J. Biol. Chem., 277(19): 16673-16681 (2002)), a human β-actin promoter, an EF-1α promoter, a YY1 promoter, a basic leucine zipper nuclear factor-1 (BLZF-1) promoter, a neuron specific enolase (NSE) promoter, a heat shock protein 70B (HSP70B) promoter, and a JEM-1 promoter.

Many of the above-described promoters are constitutive promoters. Instead of being a constitutive promoter, the promoter can be an inducible promoter, i.e., a promoter that is up- and/or down-regulated in response to an appropriate signal. The use of a regulatable promoter or expression control sequence is particularly applicable to DNA vaccine development inasmuch as antigenic proteins, including viral and parasite antigens, frequently are toxic to cell lines in which they are produced. A promoter can be up-regulated by a radiant energy source or by a substance that distresses cells. For example, a promoter can be up-regulated by drugs, hormones, ultrasound, light activated compounds, radiofrequency, chemotherapy, and cyofreezing. Thus, a promoter sequence that regulates expression of the inventive nucleic acid sequence can contain at least one heterologous regulatory sequence responsive to regulation by an exogenous agent. Suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed.

The promoter can be a tissue-specific promoter, i.e., a promoter that is preferentially activated in a given tissue and results in expression of a gene product in the tissue where activated. A tissue-specific promoter suitable for use in the invention can be chosen by the ordinarily skilled artisan based upon the target tissue or cell-type. Preferred tissue-specific promoters for use in the inventive method are specific to immune cells.

To optimize protein production, preferably the inventive nucleic acid molecule further comprises a polyadenylation site 3′ of the coding sequence. Any suitable polyadenylation sequence can be used, including a synthetic optimized sequence, as well as the polyadenylation sequence of SV40 (Human Sarcoma Virus-40), BGH (Bovine Growth Hormone), mouse globin D (MGD), polyoma virus, TK (Thymidine Kinase), EBV (Epstein Barr Virus), and the papillomaviruses, including human papillomaviruses and BPV (Bovine Papilloma Virus). Also, preferably all of the proper transcription signals (and translation signals, where appropriate) are correctly arranged such that the nucleic acid sequence is properly expressed in the cells into which it is introduced. If desired, the nucleic acid sequence also can incorporate splice sites (i.e., splice acceptor and splice donor sites) to facilitate mRNA production.

The invention also provides a polypeptide encoded by the nucleic acid molecule comprising or consisting of a nucleic acid sequence of SEQ ID NO: 1, SEQ ID NO: 2, SEQ ID NO: 3, or SEQ ID NO: 4. As discussed hererin with respect to the inventive nucleic acid molecule, the polypeptide can be modified in any suitable manner for any purpose, such as, for example, to enhance the immunogenicity of the Env or Gag polypeptide encoded thereby, or to enhance the expression of the nucleic acid sequence in vivo. For example, the Env polypeptide encodded by SEQ ID NO: 1 or SEQ ID NO: 2 can comprise mutations in the cleavage site, fusion peptide, or interhelical coiled-coil domains of a wild-type Env protein (ACFI Env proteins), and/or it can lack the cytoplasmic domain of a wild-type Env protein. The Env polypeptide also can lack one or more variable loops of a wild-type Env polypeptide. The polypeptide can be modified to increase its stability in vivo by, for example, the addition of functional groups (e.g., glycosyl groups), or by linkage to other polypeptide moeities to produce a fusion protein as described above. The polypeptide can be modified in any chemical or structural manner known in the art so as to enhance its expression, stability, and/or function in vivo. The invention also provides an isolated or purified nucleic acid molecule comprising a nucleic acid sequence encoding the aforementioned polypeptide.

The invention provides an isolated host cell comprising the nucleic acid molecule of the invention, or a construct comprising the nucleic acid molecule of the invention. For example, the nucleic acid molecule or construct can be expressed in prokaryotic cells, such as E. coli. Preferably, the nucleic acid molecule or construct is expressed in eukaryotic cells, such as insect cells (using baculovirus expression vectors), yeast cells, or mammalian cells (e.g., Chinese hamster ovary (CHO) cells, 293 cells, COS cells, or other human cells). Suitable host cells are discussed further in Goeddel, supra. Nucleic acid sequences and vectors comprising nucleic acid sequences (i.e., “constructs”) can be introduced into prokaryotic or eukaryotic cells via conventional transformation or transfection techniques. Such techniques include, for example, calcium phosphate or calcium chloride co-precipitation, DEAE-dextran-mediated transfection, lipofection, or electroporation. Suitable methods for transforming or transfecting host cells can be found in Sambrook, et al., supra. An isolated host cell, such as a prokaryotic or eukaryotic host cell in culture, can be used to produce (i.e., express) any of the HIV-1 polypeptides encoded by the nucleic acid molecules described herein.

The invention further provides a method of inducing an immune response against HIV-1 in a mammal (preferably a human). In one embodiment, the method comprises administering to a mammal the inventive nucleic acid molecule which comprises or consists of the nucleic acid sequence of SEQ ID NO: 1, SEQ ID NO: 2, SEQ ID NO: 3, or SEQ ID NO: 4 described herein. In another embodiment, the method comprises administering to a mammal a composition comprising the inventive nucleic acid molecule or construct (e.g., plasmid construct or adenoviral vector construct) described herein. In yet another embodiment, the method comprises administering to a mammal the polypeptide encoded by the inventive nucleic acid molecule described herein.

The inventive nucleic acid molecule, or a construct comprising the inventive nucleic acid molecule, desirably is administered in a composition, preferably a pharmaceutically acceptable (e.g., physiologically acceptable) composition, which comprises a carrier, preferably a pharmaceutically (e.g., physiologically acceptable) carrier and the nucleic acid molecule, construct, or polypeptide. Therefore, the invention provides a composition capable of eliciting an immune response against HIV. The composition can be capable of eliciting a protective immune response against HIV when administered alone, or in combination with at least one additional immunogenic agent or composition. It will be understood by those of skill in the art that the ability to produce an immune response after exposure to an antigen is a function of complex cellular and humoral processes, and that different subjects have varying capacity to respond to an immunological stimulus. Accordingly, the compositions disclosed herein are capable of eliciting an immune response in an immunocompetent subject, that is a subject that is physiologically capable of responding to an immunological stimulus by the production of a substantially normal immune response, e.g., including the production of antibodies that specifically interact with the immunological stimulus, and/or the production of functional T-cells (CD4⁺ and/or CD8⁺ T-cells) that bear receptors that specifically interact with the immunological stimulus. It will further be understood that a particular effect of infection with HIV is to render a previously immunocompetent subject immunodeficient. Thus, with respect to the methods discussed herein, it is generally desirable to administer the compositions to a subject prior to exposure to HIV (that is, prophylactically, e.g., as a vaccine) or therapeutically at a time following exposure to HIV during which the subject is nonetheless capable of developing an immune response to a stimulus, such as an antigenic polypeptide.

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the composition is formulated to protect the nucleic acid sequence or vector from damage prior to administration. For example, the pharmaceutical composition can be formulated to reduce loss of the nucleic acid or construct on devices used to prepare, store, or administer the composition, such as glassware, syringes, or needles. The composition can be formulated to decrease the light sensitivity and/or temperature sensitivity of the nucleic acid sequence or construct. To this end, the composition preferably comprises a pharmaceutically acceptable liquid carrier, such as, for example, those described above, and a stabilizing agent selected from the group consisting of Polysorbate 80, L-arginine, polyvinylpyrrolidone, trehalose, and combinations thereof. Use of such a composition will extend the shelf life of the nucleic acid sequence or construct, facilitate administration, and increase the efficiency of the inventive method.

A composition also can be formulated to enhance transduction efficiency of the nucleic acid molecule or construct. In addition, one of ordinary skill in the art will appreciate that the composition can comprise other therapeutic or biologically-active agents. For example, factors that control inflammation, such as ibuprofen or steroids, can be part of the composition to reduce swelling and inflammation associated with in vivo administration of the composition. Antibiotics, i.e., microbicides and fungicides, can be present to treat existing infection and/or reduce the risk of future infection, such as infection associated with gene transfer procedures.

The composition also can be formulated to contain an adjuvant in order to enhance the immunological response. Suitable adjuvants include, but are not limited to, mineral gels such as aluminum hydroxide, surface active substances such as lysolecithin, pluronic polyols, polyanions, other peptides, oil emulsions, and potentially useful human adjuvants such as Bacillus Calmette Guerin (BCG) and Corynebacterium parvum. Adjuvants for inclusion in the inventive composition desirably are safe, well tolerated, and effective in humans, such as QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (as described in, e.g., Kim et al., Vaccine, 18: 597 (2000)). Other adjuvants that can be administered to a mammal include lectins, growth factors, cytokines, and lymphokines (e.g., alpha-interferon, gamma-interferon, platelet derived growth factor (PDGF), gCSF, gMCSF, TNF, epidermal growth factor (EGF), IL-1, IL-2, IL-4, IL-6, IL-8, IL-10, and IL-12).

Any route of administration can be used to deliver the composition to the mammal. Indeed, although more than one route can be used to administer the composition, a particular route can provide a more immediate and more effective reaction than another route. Preferably, the composition is administered via intramuscular injection, for example, using a syringe or needleless delivery device. In this respect, the invention also provides a syringe or a needleless delivery device comprising the composition. The pharmaceutical composition also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the composition. The composition also can be administered in the form of a sustained-release formulation (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the composition administered to the mammal will depend on a number of factors, including the size of a target tissue, the extent of any side-effects, the particular route of administration, and the like. The dose ideally comprises an “effective amount” of the composition, i.e., a dose of composition which provokes a desired immune response in the mammal. The desired immune response can entail production of antibodies, protection upon subsequent challenge, immune tolerance, immune cell activation, and the like. One dose or multiple doses of the composition can be administered to a mammal to elicit an immune response with desired characteristics, including the production of HIV specific antibodies, or the production of functional T-cells that react with HIV. In certain embodiments, the T-cells may be CD8 T-cells.

When the inventive nucleic acid molecule is administered to a mammal via an adenoviral vector, the composition desirably comprises a single dose of adenoviral vector construct comprising at least about 1×10⁵ particles (which also is referred to as particle units) of adenoviral vector construct. The dose preferably is at least about 1×10⁶ particles (e.g., about 1×10⁶-1×10¹² particles), more preferably at least about 1×10⁷ particles, even more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector construct. Alternatively, the dose comprises no more than about 1×10¹⁴ particles, preferably no more than about 1×10¹³ particles, more preferably no more than about 1×10¹² particles, even more preferably no more than about 1×10¹¹ particles, and most preferably no more than about 1×10¹⁰ particles (e.g., no more than about 1×10⁹ particles). In other words, the composition can comprise a single dose of adenoviral vector construct comprising, for example, about 1×10⁶ particle units (pu), 2×10⁶ pu, 4×10⁶ pu, 1×10⁷ pu, 2×10⁷ pu, 4×10⁷ pu, 1×10⁸ pu, 2×10⁸ pu, 4×10⁸ pu, 1×10⁹ pu, 2×10⁹ pu, 4×10⁹ pu, 1×10¹⁰ pu, 2×10¹⁰ pu, 4×10¹⁰u, 1×10¹¹ pu, 2×10¹¹ pu, 4×10¹¹ pu, 1×10¹² pu, 2×10¹² pu, or 4×10¹² pu of adenoviral vector construct.

Administration of the inventive nucleic acid molecule, composition, or polypeptide can be one component of a multistep regimen for inducing an immune response against HIV in a mammal. In particular, the inventive method can represent one arm of a prime and boost immunization regimen. The inventive method, therefore, can comprise administering to the mammal any suitable “priming” composition prior to administering the inventive nucleic acid molecule, composition, or polypeptide. Thus, in this embodiment, an immune response is “primed” by administration of the priming composition, and is “boosted” by administration of the inventive nucleic acid molecule, composition, or polypeptide. Alternatively, the inventive method can comprise administering to the mammal any suitable “boosting” composition following administration of the inventive nucleic acid molecule, composition, or polypeptide. Thus, in this embodiment, an immune response is “primed” by administration of the inventive nucleic acid molecule, composition, or polypeptide, and is “boosted” by administration of the boosting composition. When the priming composition or boosting composition is not the inventive nucleic acid molecule, composition, or polypeptide, the priming composition or boosting composition desirably comprises one or more nucleic acid molecules that encode at least one HIV polypeptide that is the same as the HIV polypeptide (e.g., an HIV-1 Env polypeptide) encoded by the inventive nucleic acid molecule.

The one or more nucleic acid molecules of the priming composition or the boosting composition can be administered as part of a vector or as naked DNA. Any vector, such as those described herein, can be employed in the priming or boosting composition, including viral and non-viral vectors. Examples of suitable viral vectors include, but are not limited to, retroviral vectors, adeno-associated virus vectors, vaccinia virus vectors, herpesvirus vectors, and adenoviral vectors. Examples of suitable non-viral vectors include, but are not limited to, plasmids, liposomes, and molecular conjugates (e.g., transferrin). Ideally, the vector is a plasmid or an adenoviral vector. Alternatively, an immune response can be primed or boosted by administration of the antigen itself, e.g., an antigenic protein, inactivated pathogen, and the like.

The priming composition is administered to the mammal to prime the immune response to HIV, while the boosting composition is administered to enhance or augment the immune response induced by the priming composition. More than one dose of the priming composition or boosting composition can be provided in any suitable timeframe. Administration of the priming composition and administration of the boosting composition desirably is separated by at least about 1 week (e.g., at least about 1 week, 2 weeks, 4 weeks, 8 weeks, 12 weeks, 16 weeks, or more). Preferably, the primer composition is administered to the mammal at least three months (e.g., three, six, nine, twelve, or more months) before administration of the boosting composition. Most preferably, the primer composition is administered to the mammal at least about six months to about nine months before administration of the boosting composition.

The following examples further illustrate the invention but, of course, should not be construed as in any way limiting its scope.

### Example 1

This example demonstrates a method of producing a mosaic HIV Gag polypeptide comprising an insertion of at least one T-cell epitope that is not naturally present in the Gag polypeptide.

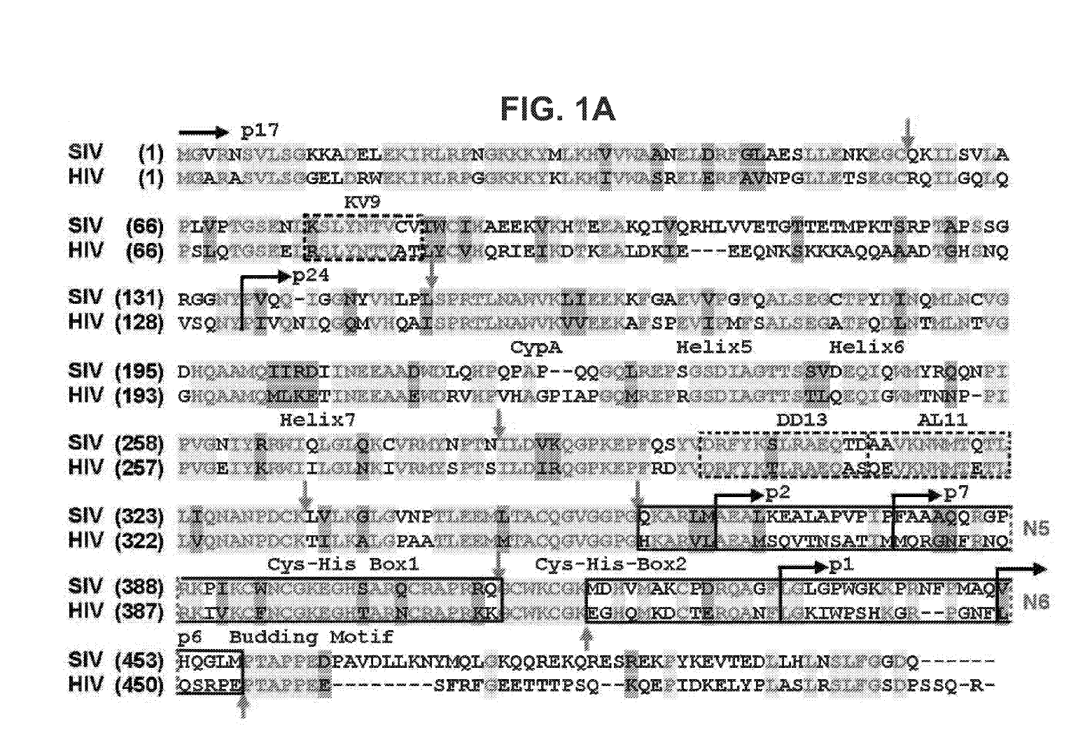

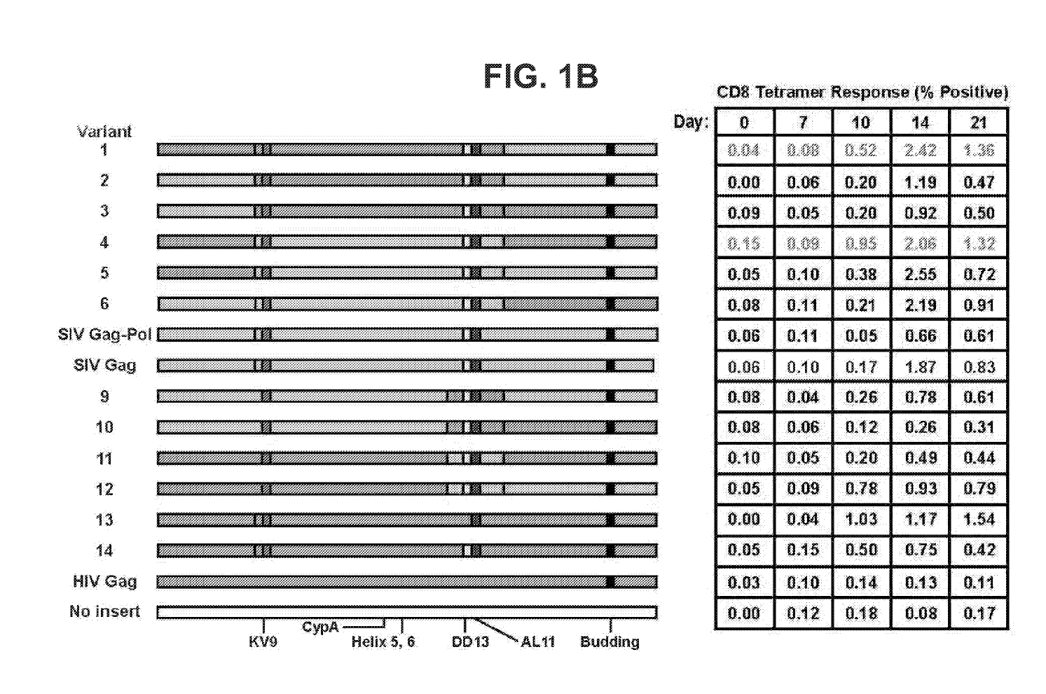

Plasmid constructs encoding chimeric HIV/SIV Gag polypeptides were generated containing HIV and SIV nucleic acid sequences of various sizes (see FIG. 1B). Wild-type Gag genes included HIV Gag, SIV Gag-Pol (SEQ ID NO: 41), and SIV Gag (SEQ ID NO: 42), and the HIV/SIV chimeric sequences included variants 1-6, variants 9-14, variant N2-variant N11, and variant N14 (see FIG. 1B and Table 1). Nucleic acid sequences encoding the KV9, DD13 and AL11 T cell epitopes from SIV Gag were introduced into the chimeras, and AL11 was used as the primary determinant for analysis of the immune response.

TABLE 1

| HIV/SIV | | | |
|---|---|---|---|
| Gag | | | Nucleic Acid Sequence |
| Variant in | Laboratory | Gag Variant Nucleic | of Plasmid Construct |
| FIG. 1B | Designation | Acid Sequence | Encoding Gag Variant |
| 1 | VRC 4733 | SEQ ID NO: 35 | SEQ ID NO: 49 |
| 2 | VRC 4734 | SEQ ID NO: 36 | SEQ ID NO: 50 |
| 3 | VRC 4735 | SEQ ID NO: 37 | SEQ ID NO: 51 |
| 4 | VRC 4736 | SEQ ID NO: 38 | SEQ ID NO: 52 |
| 5 | VRC 4737 | SEQ ID NO: 39 | SEQ ID NO: 53 |
| 6 | VRC 4738 | SEQ ID NO: 40 | SEQ ID NO: 54 |
| SIV Gag-Pol | VRC 4739 | SEQ ID NO: 41 | SEQ ID NO: 55 |
| SIV Gag | VRC 4740 | SEQ ID NO: 42 | SEQ ID NO: 56 |
| 9 | VRC 4741 | SEQ ID NO: 43 | SEQ ID NO: 57 |
| 10 | VRC 4742 | SEQ ID NO: 44 | SEQ ID NO: 58 |
| 11 | VRC 4743 | SEQ ID NO: 45 | SEQ ID NO: 59 |
| 12 | VRC 4744 | SEQ ID NO: 46 | SEQ ID NO: 60 |
| 13 | VRC 4745 | SEQ ID NO: 47 | SEQ ID NO: 61 |
| 14 | VRC 4746 | SEQ ID NO: 48 | SEQ ID NO: 62 |

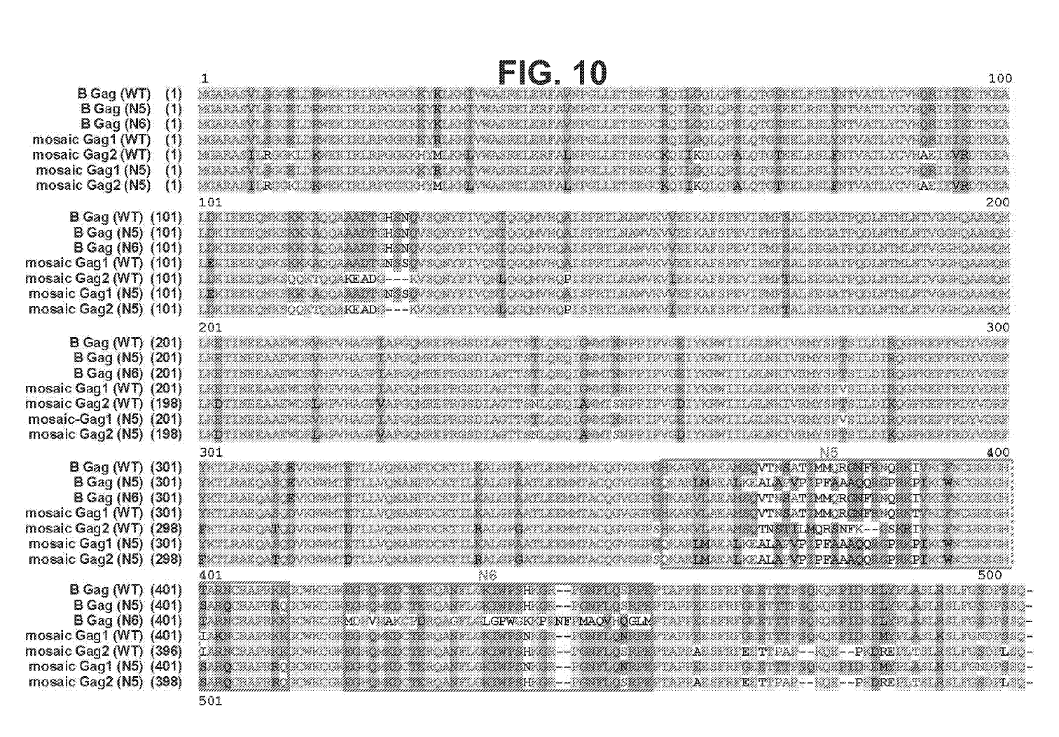

After an initial immune enhancing region determination was made, an “N5-modified” Gag (B Gag N5) polypeptide and an “N6-modified” Gag (B Gag N6) polypeptide were generated by introducing a nucleic acid sequence encoding the SIV N5 amino acid region (SEQ ID NO: 27) or a nucleic acid sequence encoding the N6 amino acid region (SEQ ID NO: 28) (indicated in FIG. 10) into the corresponding region of a wild-type HIV-1 Gag gene. In addition, a set of two wild-type informatic HIV mosaic Gag genes (mosaic Gag1 (WT) (SEQ ID NO: 16) and mosaic Gag2(WT) (SEQ ID NO: 17)) and a set of two chimeric N5-modified HIV informatic mosaic Gag genes (mosaic Gag1 (N5) (SEQ ID NO: 18) and Mosaic Gag2(N5) (SEQ ID NO: 19)) were evaluated.

Informatic mosaic Gag and Env proteins were designed using the methods described in Fischer et al., Nat. Med., 13: 100-106 (2007). A web-based suite of tools is available that enables generation of candidate mosaic sequences for any set of variable pathogen proteins, and epitope length sequence coverage comparison of different vaccine antigen candidates (Thurmond et al., Bioinformatics, 24: 1639-1640 (2008)). The Gag mosaics were optimized as a set of 2 mosaic Gag genes which were selected with optimization criteria described previously (see, e.g., Gao et al., Science, 299: 1517-1518 (2003); Gaschen et al., Science, 296: 2354-2360 (2002); Gautam et al., Virology, 362: 257-270 (2007); and Kawada et al., J. Virol., 82: 10199-10206 (2008)). The full length amino acid sequences of the wild-type Gag (SEQ ID NO: 20), the N5-modified Gag (SEQ ID NO: 21), the N6 chimeric Gag (SEQ ID NO: 22), the set of two wild-type informatic HIV mosaic Gag genes (mosaic Gag1(WT) (SEQ ID NO: 23) and mosaic Gag2(WT) (SEQ ID NO: 24)), and the set of two N5-modified chimeric mosaic Gag genes ((mosaic Gag1(N5) (SEQ ID NO: 25) and Mosaic Gag2(N5) (SEQ ID NO: 26)) are shown in FIG. 10.

All modified HIV Gag genes were synthesized using human-preferred codons (GeneArt, Regensburg, Germany) (see, e.g., Kong et al., Proc. Natl. Acad. Sci. USA, 103: 15987-15991 (2006)) or by preparation of oligonucleotides of (i) 75 base pairs (bp) overlapping by 25 or (ii) 60 bp overlapping by 20 and assembled by Pwo DNA polymerase (Boehringer Mannheim, Germany) and Turbo Pfu® (Stratagene, La Jolla, Calif.) as described previously (see, e.g., Chakrabarti et al., J. Virol., 76: 5357-5368 (2002), and Kong et al., J. Virol., 77: 12764-12772 (2003)). All deletions or other modifications were generated by site-directed mutagenesis using a QuickChange kit (Stratagene, La Jolla, Calif.) or by overlapping PCR. The cDNAs were cloned into a plasmid expression vector, pCMV/R, which mediates high level expression and immunogenicity in vivo (see, e.g., Barouch et al., J. Virol., 79: 8828-8834 (2005), and Yang et al., Science, 317: 825-828 (2007)).

Replication-defective serotype 5 adenoviral vector constructs (rAd) comprising nucleic acid sequence encoding wild-type Gag (B Gag(WT) (SEQ ID NO: 13)), the N5-modified Gag (B Gag(N5) (SEQ ID NO; 14)), the N6-modified Gag (B Gag(N6) (SEQ ID NO: 15)), two wild-type mosaic Gags (mosaic Gag1(WT) (SEQ ID NO: 16) and mosaic Gag2(WT) (SEQ ID NO: 17)), two N5-modified chimeric mosaic Gags genes ((mosaic Gag1 (N5) (SEQ ID NO: 18) and Mosaic Gag2(N5) (SEQ ID NO: 19)), the N6-modified chimeric mosaic Gag gene (mosaic Gag(N6) (SEQ ID NO: 15)), and a set of mosaic Envs (mosaic Env) genes (SEQ ID NO: 1 and SEQ ID NO: 2) were produced as previously described (see, e.g., Wu et al., J. Virol., 79: 8024-8031 (2005)).

The plasmid constructs and adenovirus constructs described above were tested for their expression in 293T and A549 cells. Plasmid constructs encoding the variant Gag proteins were transferred into 293T cells using the calcium phosphate-mediated ProFection® Mammalian Transfection system (Promega, Madison, Wis.). Adenovirus constructs encoding the variant Gag proteins were infected into A549 cells for 48 hours followed by a change of media. Cell lysates were collected 48 hours post-infection and subjected to Western blot analysis by human anti HIV polyclonal serum and anti SIV P27 polyclonal serum (Immune Technology Corp., New York, N.Y.). Specific bands of the predicted size of proteins were detected by comparison to a known vector control. The 293T transfected cells also were determined by electron-microscopy to confirm appearance of the Gag formation particles (see e.g., Wataru et al., J. Virol., 79:626-631 (2005)).

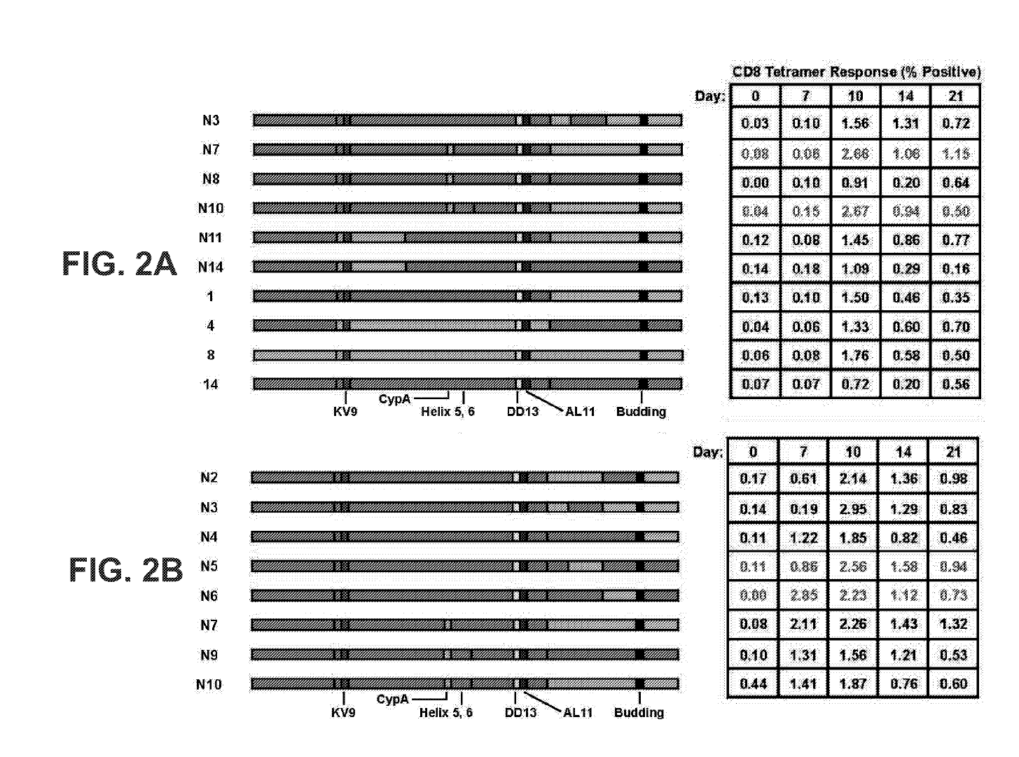

Groups of C57BL/6 mice were immunized intramuscularly with 50 μg of the plasmid constructs described above. PBMC from immunized mice were collected at days 0, 7, 10, 14 and 21 after immunization. The T cells were subjected to D^{b}/AL11-specific tetramer binding assays as previously described (Mascola et al., J. Virol., 77: 10348-10356 (2003)). The highest CD8 AL11 tetramer response was elicited by two plasmid constructs as determined by the AL11+-specific CD8 tetramer response (FIG. 1B, variants 1 and 4). Based on localization of these domains in the middle or COOH-terminus of SIV Gag, additional subregions were analyzed, of which two plasmid constructs encoding Gag segments in the COOH-terminus showed similar enhanced T cell responses compared to HIV-1 Gag (FIGS. 2A and 2B, variants N7 and N10). Additional mapping was performed with smaller SIV Gag chimeric segments (see Table 2).

TABLE 2

| HIV/SIV | | | |
|---|---|---|---|
| Gag Variant | | Nucleic Acid | Nucleic Acid Sequence |
| in FIGS. | Laboratory | Sequence of Gag | of Plasmid Construct |
| 2A and 2B | Designation | Variant | Encoding Gag Variant |
| N2 | VRC 4714 | SEQ ID NO: 70 | SEQ ID NO: 71 |
| N3 | VRC 4715 | SEQ ID NO: 72 | SEQ ID NO: 73 |
| N4 | VRC 4716 | SEQ ID NO: 74 | SEQ ID NO: 75 |
| N5 | VRC 4717 | SEQ ID NO: 76 | SEQ ID NO: 77 |
| N6 | VRC 4718 | SEQ ID NO: 78 | SEQ ID NO: 79 |
| N7 | VRC 4719 | SEQ ID NO: 80 | SEQ ID NO: 81 |
| N8 | VRC 4720 | SEQ ID NO: 82 | SEQ ID NO: 83 |
| N9 | VRC 4721 | SEQ ID NO: 84 | SEQ ID NO: 85 |
| N10 | VRC 4722 | SEQ ID NO: 86 | SEQ ID NO: 87 |
| N11 | VRC 4723 | SEQ ID NO: 88 | SEQ ID NO: 89 |
| N14 | VRC 4726 | SEQ ID NO: 94 | SEQ ID NO: 95 |
| 1 | VRC 4733 | SEQ ID NO: 35 | SEQ ID NO: 49 |
| 4 | VRC 4736 | SEQ ID NO: 38 | SEQ ID NO: 52 |
| 8 | VRC 4740 | SEQ ID NO: 42 | SEQ ID NO: 56 |
| 14 | VRC 4746 | SEQ ID NO: 48 | SEQ ID NO: 62 |

In the COOH-terminal region, two chimeric Gag sequences continued to elicit increased AL11+ CD8 tetramer responses (FIG. 2B, variants N5 and N6). N5 included 60 amino acids of SIV Gag (aa 358 to 418, SEQ ID NO: 30), while N6 encoded 43 amino acids (aa 419 to 462, SEQ ID NO: 31). The primary amino acid differences and their relationship to known structural motifs in Gag are shown (FIG. 10). Expression of relevant Gag chimeric proteins was confirmed by Western blotting using HIV-1+ human serum and anti-SIV serum.

This example demonstrates a method of producing a nucleic acid sequence encoding an HIV-1 Gag polypeptide and a nucleic acid sequence encoding an Env polypeptide comprising an insertion of at least one SIV T-cell epitope that is not naturally present the HIV Gag or Env polypeptide, as well as constructs comprising such nucleic acid sequences.

### Example 2

This example describes the immunogenicity of an N5-modified Gag chimeric polypeptide as compared to a wild-type Gag polypeptide by intracellular cytokine staining.

Two chimeric HIV/SIV Gag polypeptides, Gag N5 and Gag N6, were analyzed further in comparison to wild-type HIV-1 Gag by ELISPOT using potential T-cell epitope (PTE) peptides designed to react with the most abundant T-cell targets of CTL recognition. Groups of five C57BL/6 mice were injected intramuscularly with 50 mg of a plasmid construct per injection at one time point followed by one boost with 1×10⁹ particles of an adenoviral vector construct (rAd) expressing the same protein as the plasmid construct four weeks later. Each of the plasmid constructs and adenoviral vector constructs contained one of the following DNA insert sequences: wild-type clade B HIV-1 Gag (B Gag(WT) (SEQ ID NO: 13)), N5-modified HIV Gag (B Gag(N5) (SEQ ID NO: 14)), and N6-modified HIV Gag (B Gag(N6) (SEQ ID NO: 15)). Splenocytes from the mice were isolated and gamma interferon (IFN-γ) enzyme-linked immunospot (ELISPOT) assays were performed on the individual samples four weeks after the rAd immunization. In particular, 320 Gag potential T-cell epitopes (PTE) 15-mer peptides were grouped into 32 pools (10 peptides in each pool). The number of spot-forming cells (SFC) per 10⁶ cells was determined with 25 SFC per 10⁶ cells as the minimal threshold response.

Immunization with plasmid constructs and adenoviral vector constructs encoding N5- and N6-modified Gag chimeric polypeptides elicited similar ELISPOT responses against pools 1, 3, 4 and 6 to HIV-1 Gag. In addition, the constructs expressing N5-modified HIV Gag elicited detectable low T cell responses to pools 5, 18, 22 and 29, while the constructs expressing N6-modified HIV Gag only responded to pool 21 even though these responses showed no statistically difference. Thus, the N5-modified HIV Gag protein showed responsiveness to a larger number of subdominant epitopes compared to N6-modified HIV Gag or wild-type Gag in this inbred mouse strain.

The immunogenicity of the N5-modified HIV/SIV Gag polypeptide was compared to the immunogenicity of wild-type HIV Gag by intracellular cytokine staining (ICS) after immunization with a plasmid construct encoding these genes as a prime, followed by administration of an adenoviral vector construct encoding these genes as a boost.

For the ICS analysis, 15-mer PTE peptides (see, e.g., Li et al., Vaccine, 24: 6893-6904 (2006)) were used to evaluate the plasmid and adenoviral vector constructs as the common standardized panel of HIV-1 peptides for T-cell-based vaccines. 492 Env and 320 Gag peptide sequence sets were designed to permit expression of the potential T-cell epitopes (PTE) found most frequently in the sequences of circulating worldwide HIV-1 strains, based on 549 full-length HIV-1 genome sequences obtained from the Los Alamos National Laboratory (LANL) HIV sequence database as of February 2005. All synthesized peptides (NIH AIDS Research and Reference Reagent Program) are 15 amino acids in length with naturally occurring 9 amino acid sequences that are potential T-cell determinants captured in an unbiased manner (see, e.g., Li et al., supra, and Malhotra et al., J. Virol., 81: 5225-5237 (2007)). The 320 Gag PTE peptides were tested individually, and also were grouped into 32 pools of 10 PTE peptides such that the peptides that carried the highest frequency 9-mers were grouped in the first pool, continuing so that the peptides with the rarest 9-mers were in the 32nd pool. Each individual Gag PTE peptide was designated in this study as the number of the original Gag PTE number followed by its Gag position in amino acid number relative to HXB2 position. The 492 Env PTE peptides were grouped into 82 pools containing 6 peptides each with the same grouping criteria as the Gag PTE. Some individual Gag PTEs and Env PTEs also were selected to be tested individually. Pooled sets of peptides, 15-mers overlapping by 11, corresponding to each of the three Envelopes included in the Env ABC polyvalent vaccine, were also used as previously described (see, e.g., Barouch et al., J. Virol., 79: 8828-8834 (2005); Catanzaro et al., Vaccine, 25: 4085-4092 (2007); Chakrabarti, et al., J. Virol., 76: 5357-5368 (2002); Fischer et al., Nat. Med., 13: 100-106 (2007); Kong et al., J. Virol., 77: 12764-12772 (2003); and Seaman, J. Virol., 79: 2956-2963 (2005)). In addition, 127 B-Gag peptides were used which cover the whole HIV-1 gag protein with 15-mer peptides with 11 amino acids overlapping for intracellular cytokine staining (ICS) stimulation as described previously (Catanzaro et al., supra; Kong et al., J. Virol., 77: 12764-12772 (2003); Kong et al., J. Virol., 83: 2201-2215 (2009); Wu et al., J. Virol., 79: 8024-8031 (2005); and International Patent Application Publication WO 2010/042817).

B6D2F1 (H2 Haplotype b/d) mice were injected three times with the plasmid constructs described above followed by the adenoviral vectors described above at two week intervals. To map the epitope-specific response, the 127 individual 15-mer HIV Gag peptides described above were analyzed. The N5-modified chimeric Gag polypeptide elicited similar magnitude and breadth of T cell responses to CD4 and CD8 epitopes compared to the wild-type Gag polypeptide in this B6D2F1 mice after immunization by the plasmid construct prime/adenoviral vector construct boost regimen.

The results of this example demonstrate that an N5-modified Gag chimeric polypeptide does not exhibit enhanced immunogenicity as compared to a wild-type HIV Gag polypeptide.

### Example 3

This example describes the immunogenicity of N5-modified Gag chimeric mosaic polypeptides as compared to a wild-type Gag mosaic polypeptide.

The N5 modification was introduced into a previously described mosaic Gag polypeptide (see in Example 1), and the magnitude and effect of this sequence on epitope-specific T-cell responses was determined. A set of two mosaic wild-type HIV Gag genes, mosaic Gag1 (WT) (SEQ ID NO: 16) and mosaic Gag2(WT) (SEQ ID NO: 17) were generated using a similar informatic approach as described for mosaic HIV Env (Kong et al., J. Virol., 83: 2201-2215 (2009)). The N5-modified chimeras of these two mosaic HIV Gag genes (i.e., mosaic Gag1 (N5) (SEQ ID NO: 18) and mosaic Gag2(N5) (SEQ ID NO: 19)) were then synthesized (FIG. 10).

T-cell responses elicited by constructs containing the wild-type mosaic Gag genes (mosaic Gag1 (WT) (SEQ ID NO: 16) or mosaic Gag2(WT) (SEQ ID NO: 17)), and constructs containing the N5-modified mosaic Gag genes (mosaic Gag1 (N5) (SEQ ID NO: 18) or mosaic Gag2(N5) (SEQ ID NO: 19)) were compared by ICS (Catanzaro et al., supra; Kong et al., J. Virol., 77: 12764-12772 (2003); Kong et al., J. Virol., 83: 2201-2215 (2009); Wu et al., J. Virol., 79: 8024-8031 (2005); and International Patent Application Publication WO 2010/042817). Briefly, mice (18 or 12 per group) were immunized once with 10¹⁰ particle units (pu) of a replication-defective serotype 5 adenoviral vector construct (rAd) containing the above-described Gag genes, or a total of 15 μg of a plasmid construct (100 μL in PBS) containing the above-described Gag genes three times at two-week intervals followed by a boost with 10¹⁰ pu of the adenoviral vector construct. Splenocytes from the same groups of mice were isolated, pooled together and intracellular cytokine staining (ICS) assays were performed on the pooled samples three weeks after the single rAd immunization or two weeks after the plasmid construct prime/rAd boost. Immunizations were administered bilaterally into the muscle of the hind leg using a needle and syringe.

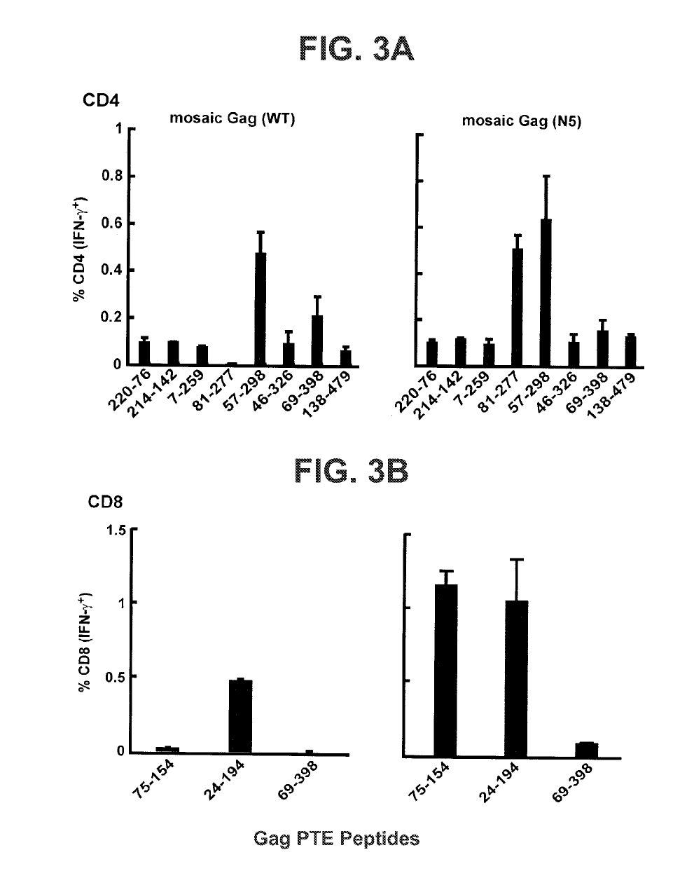

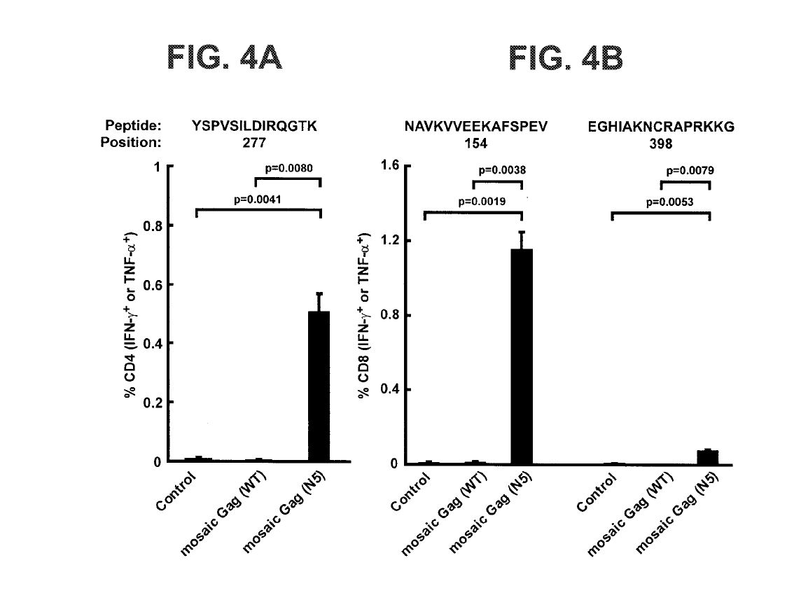

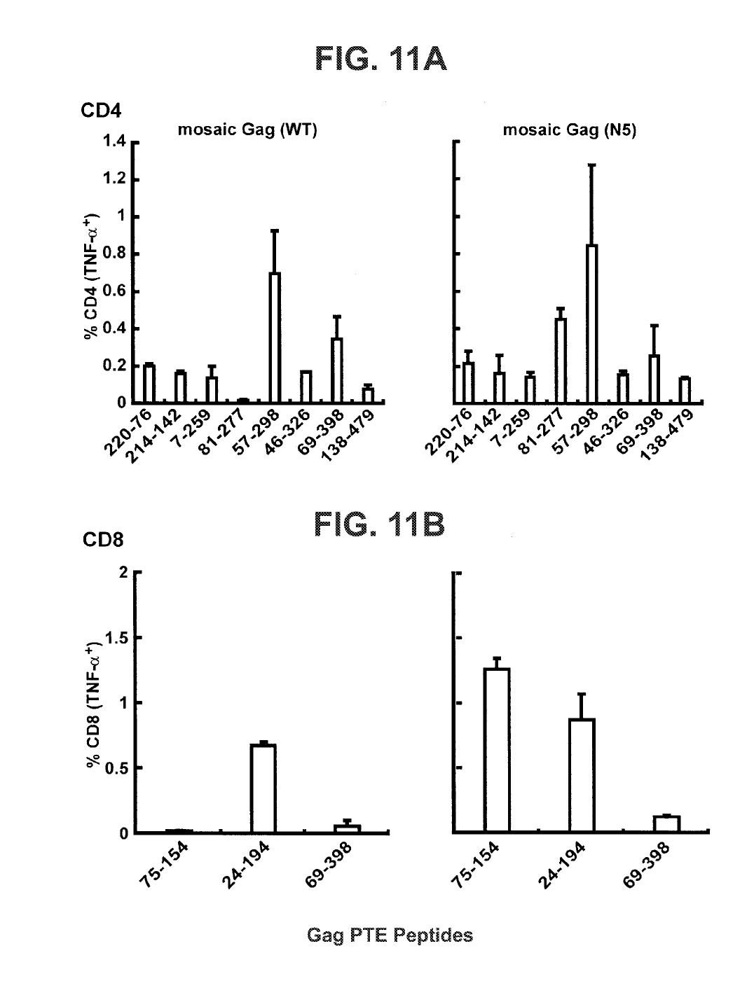

For ICS analysis, 320 individual PTE peptides were used. Immunization using the plasmid construct as a prime and the rAd5 vector construct as a boost elicited similar CD4+ (FIG. 3A) and CD8+ (FIG. 3B) T-cell responses to a few individual 320 Gag PTE peptides. The CD4 responses elicited by the mosaic Gag and N5-modified mosaic Gag constructs were similar (FIG. 3A). The peptide 57-298 was the common dominant CD4 epitope, and six other weak subdominant epitopes were found. N5-modified mosaic Gag constructs elicited one additional CD4 epitope (at aa 277 in Gag PTE #81) with a significant high response which was not found in the wild-type (FIG. 3A and FIG. 4A). In contrast, the CD8 responses elicited by the mosaic Gag and N5-modified mosaic Gag constructs were similar, as only one common CD8 epitope was identified at aa 194 of Gag PTE #24. In addition, the N5-modified mosaic Gag constructs elicited two additional epitopes not found in the wild-type, at aa154 of Gag PTE#75 and at aa 398 of Gag PTE #69 (FIG. 3B and FIG. 4B). TNF-α response analysis confined the results of the IFN-γ responses (FIGS. 11A and 11B). These results suggest that the N5 modification of HIV mosaic Gag proteins elicits both CD4+ and CD8+ T cell responses to additional epitopes as compared to wild-type mosaic Gag in B6D2F1 mice after gene-based vaccination.

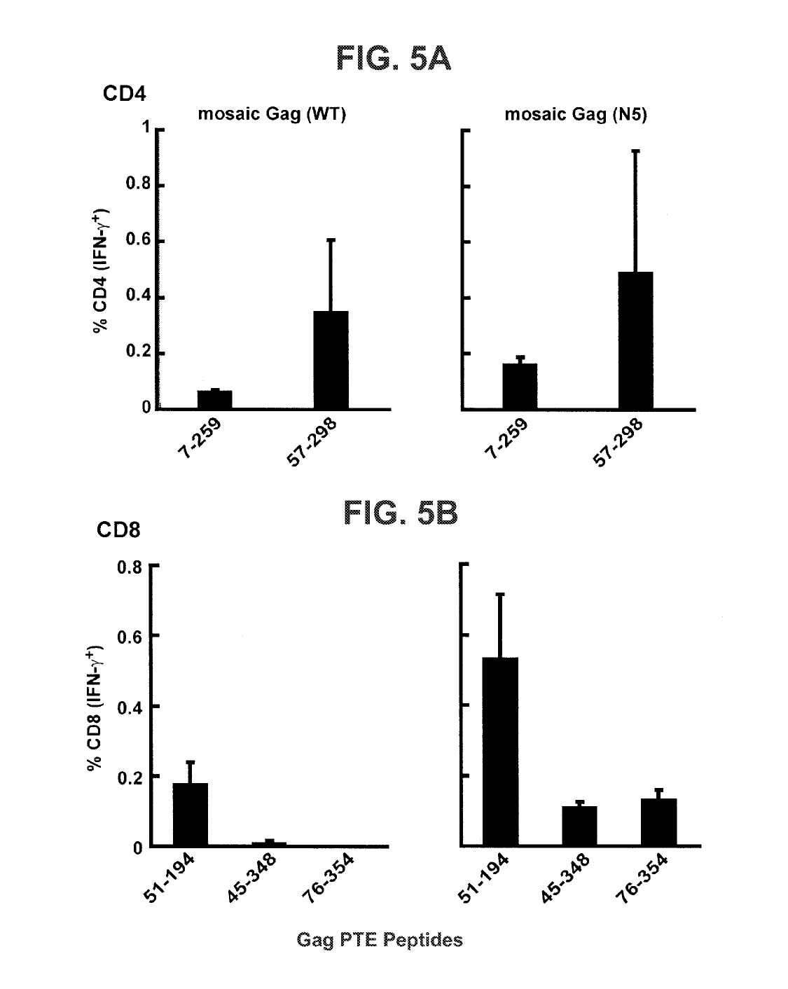

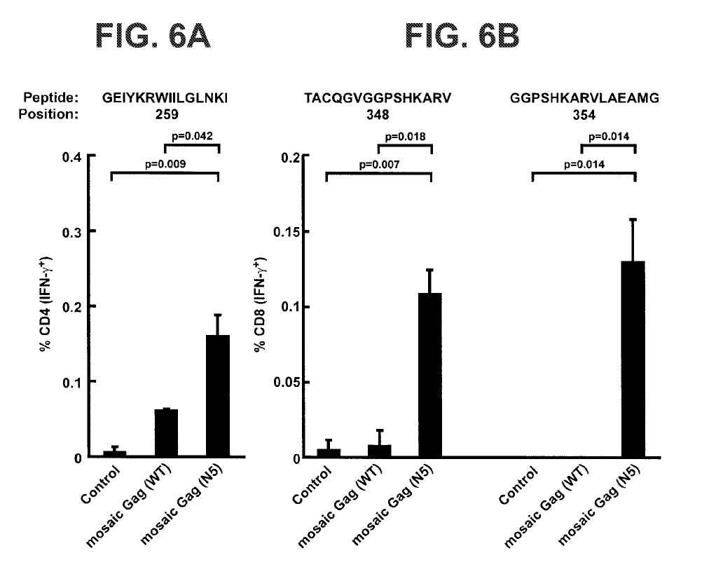

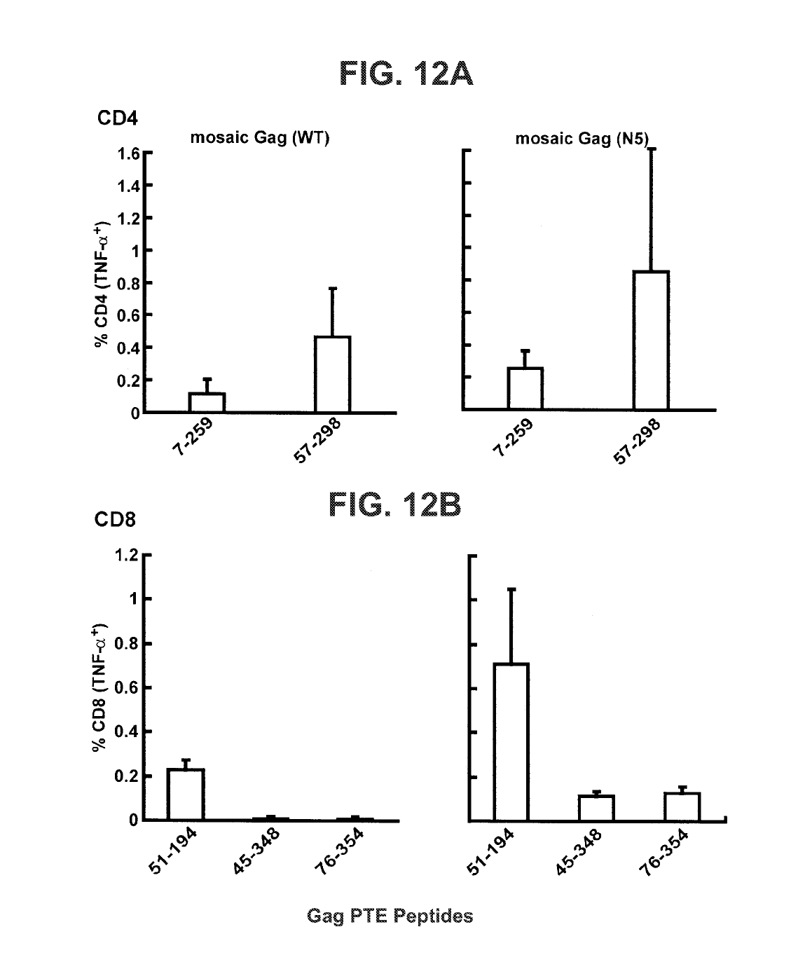

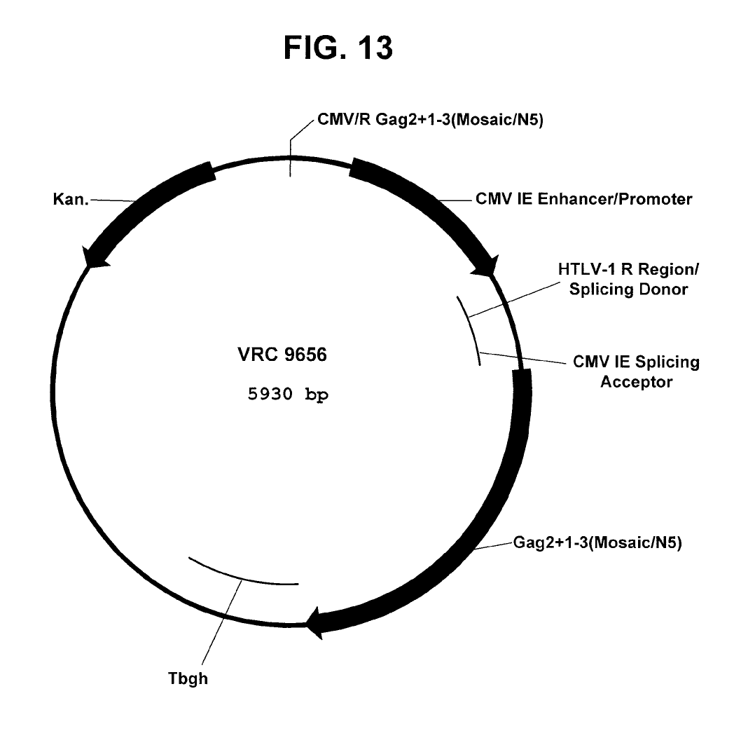

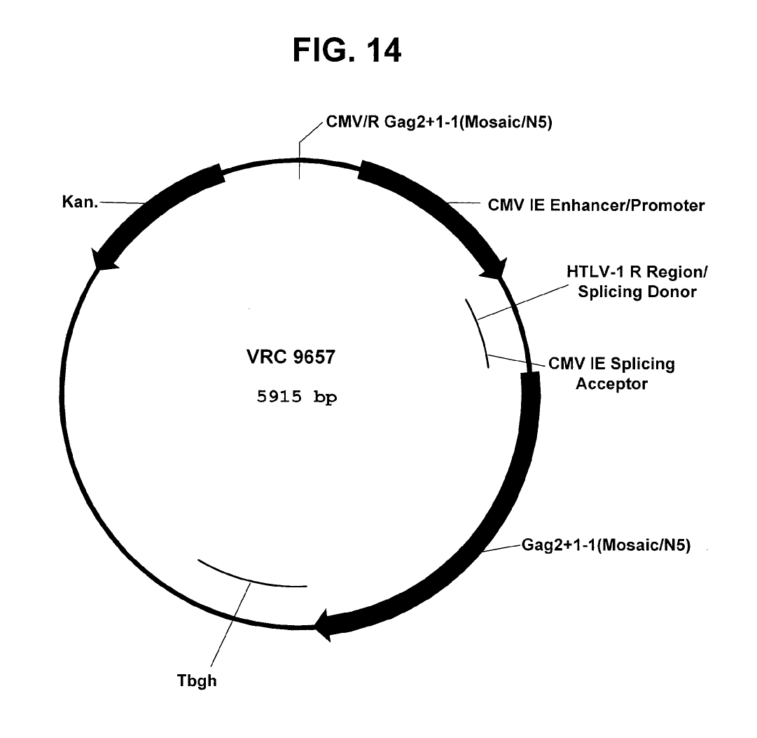

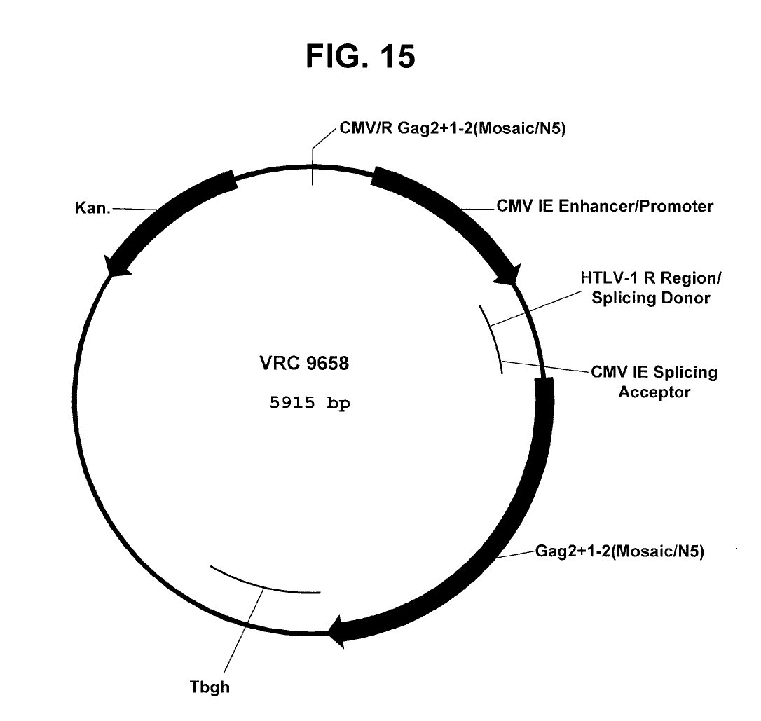

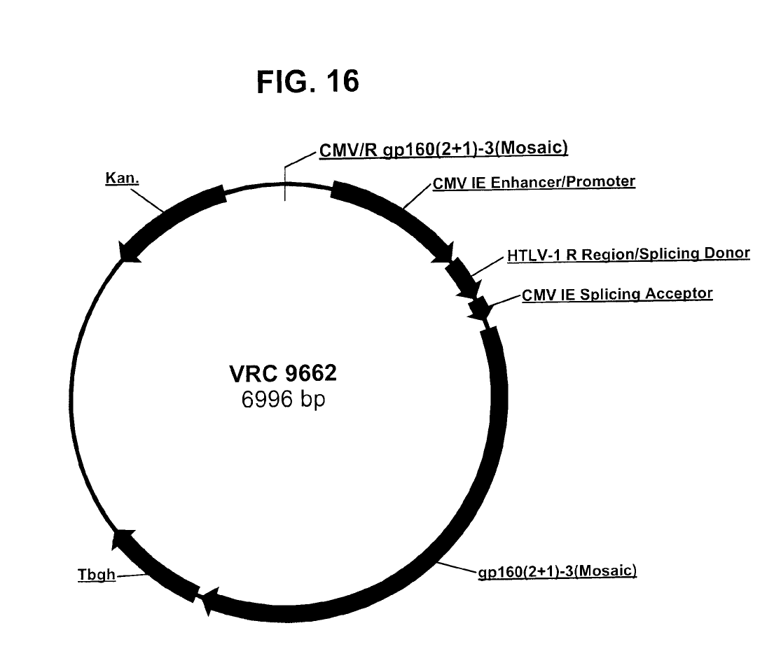

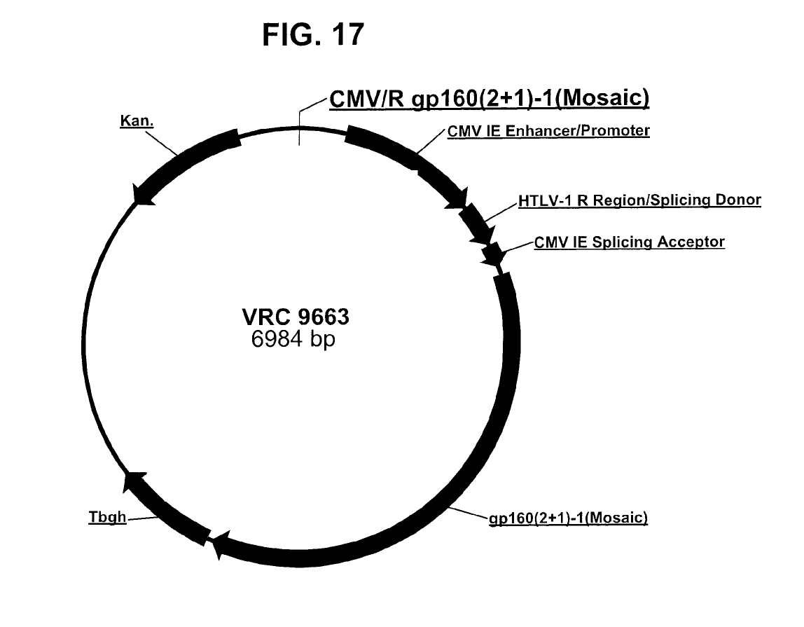

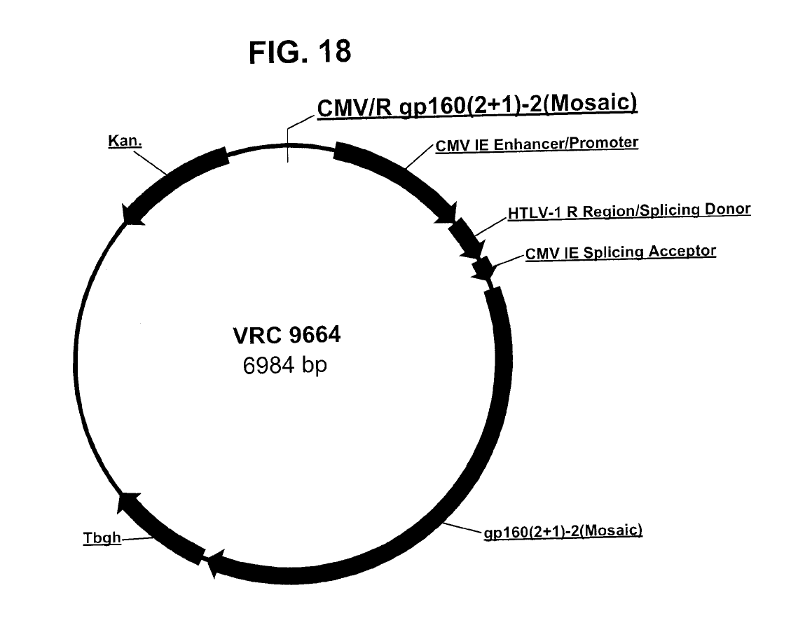

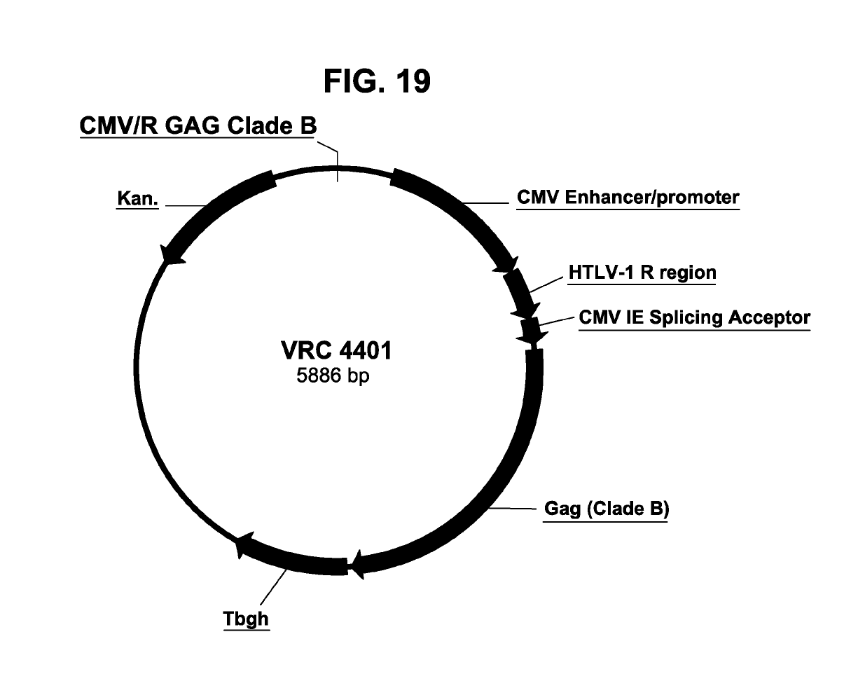

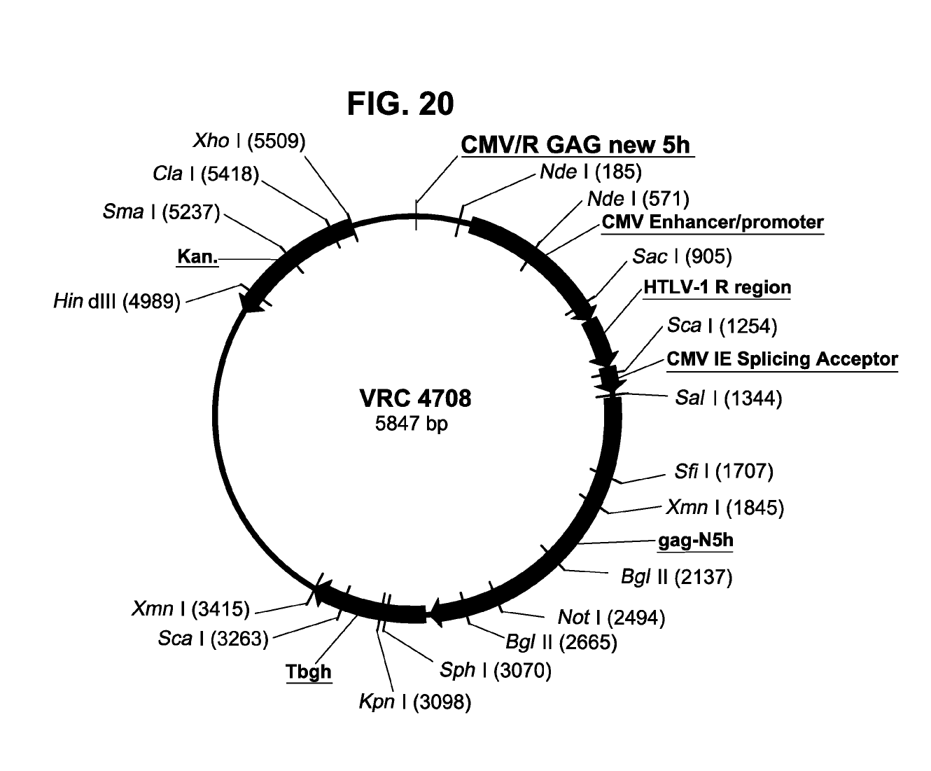

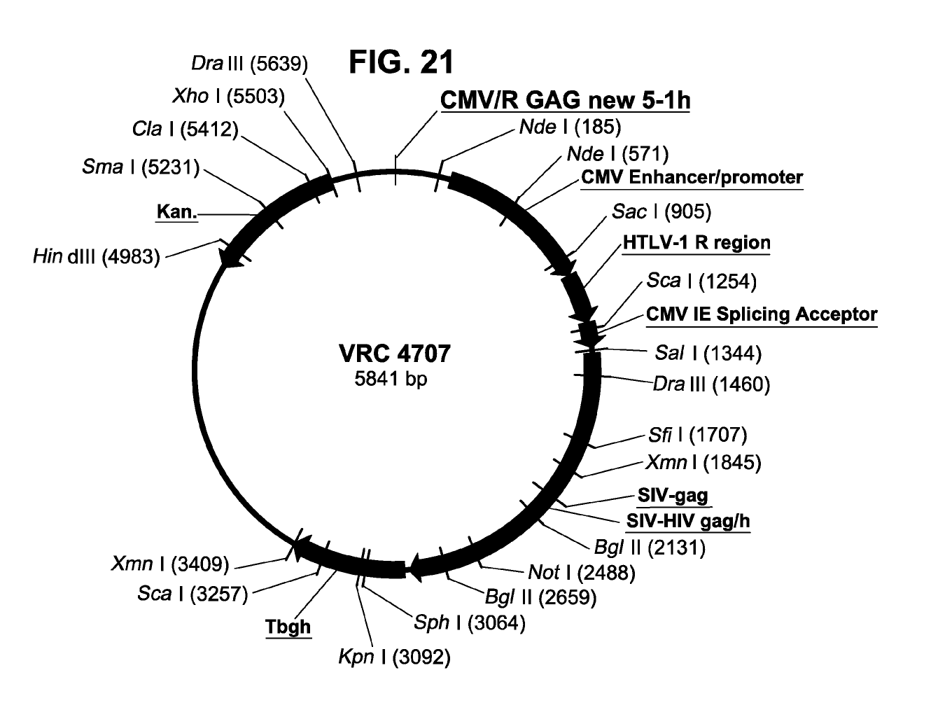

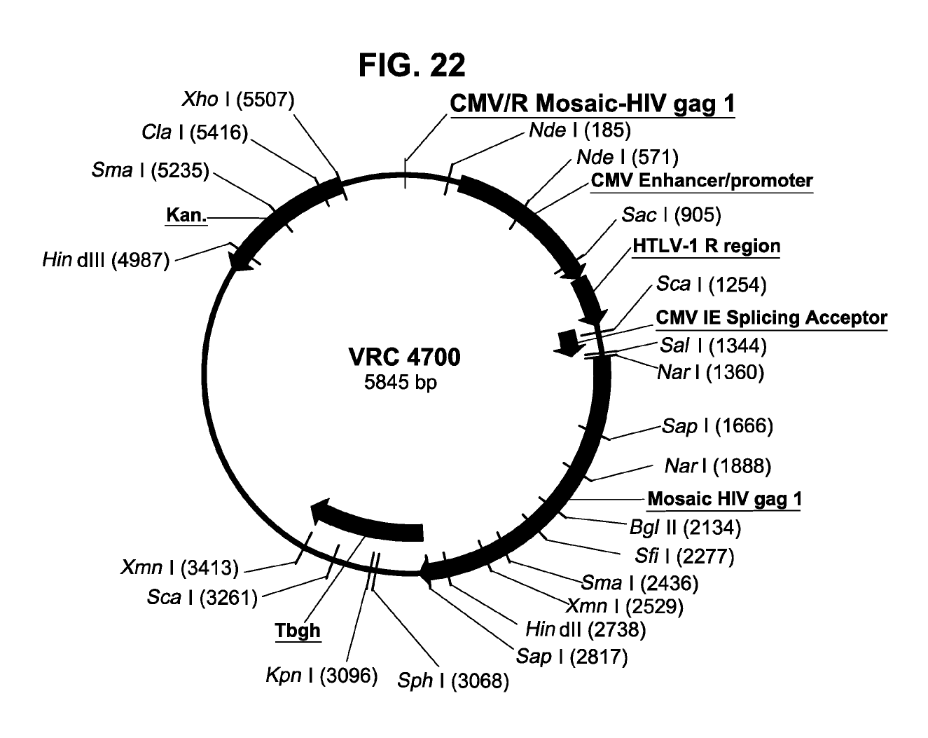

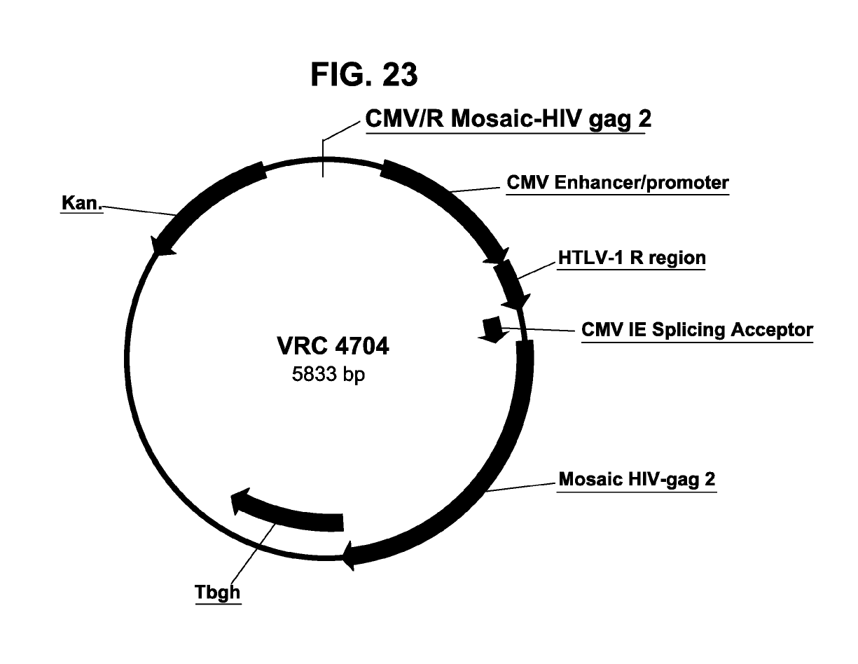

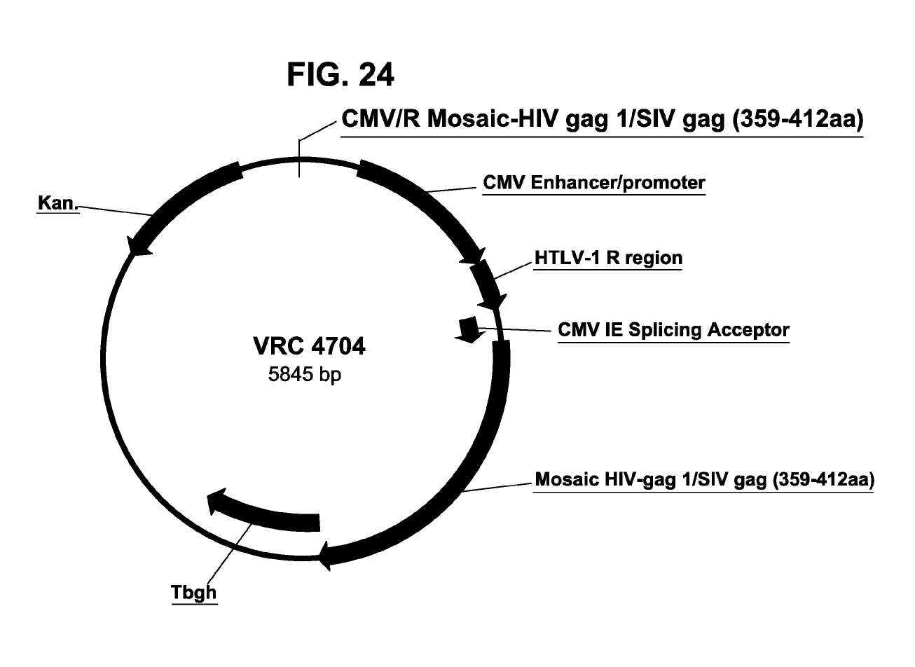

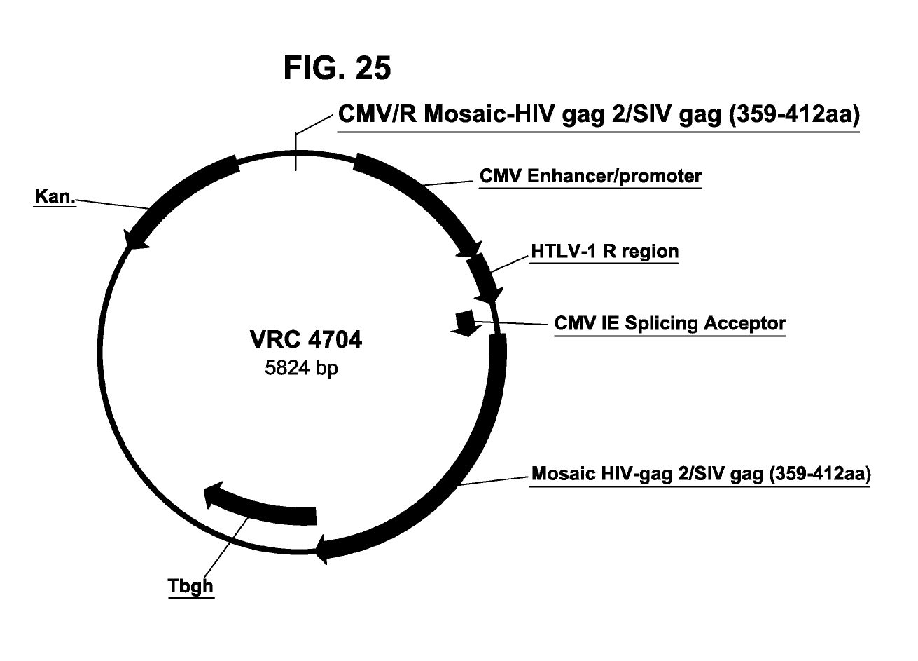

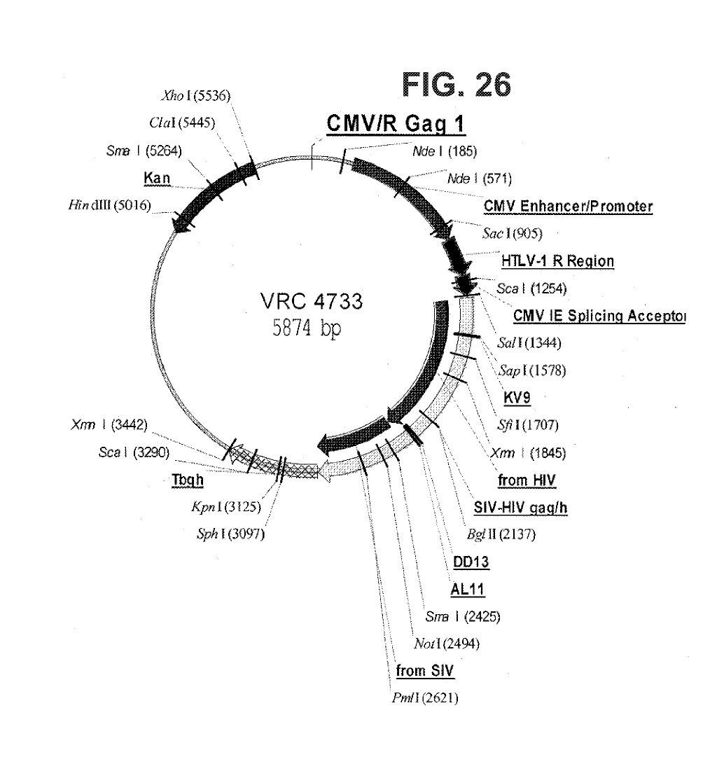

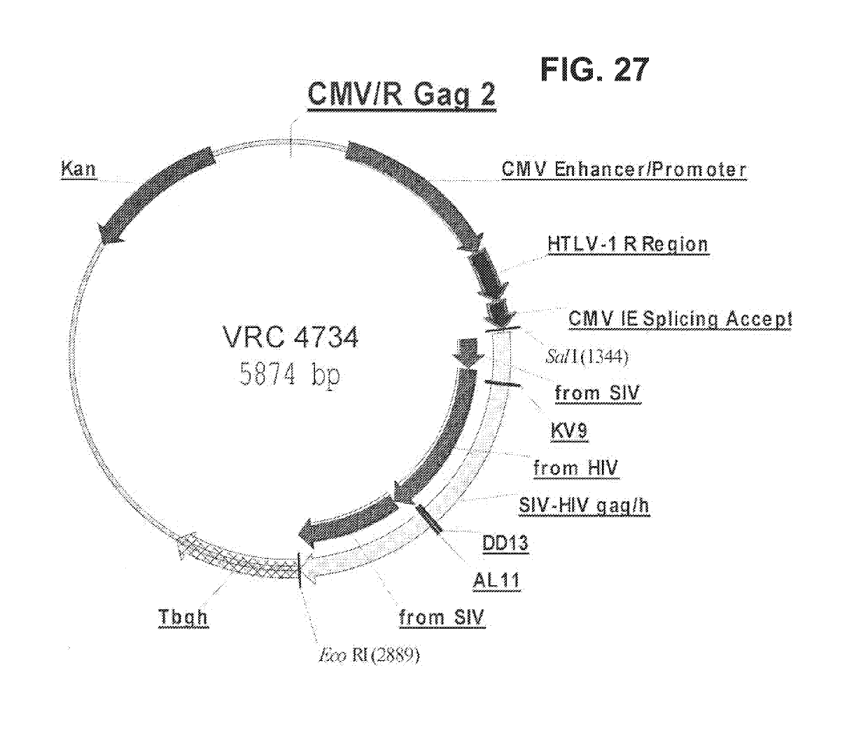

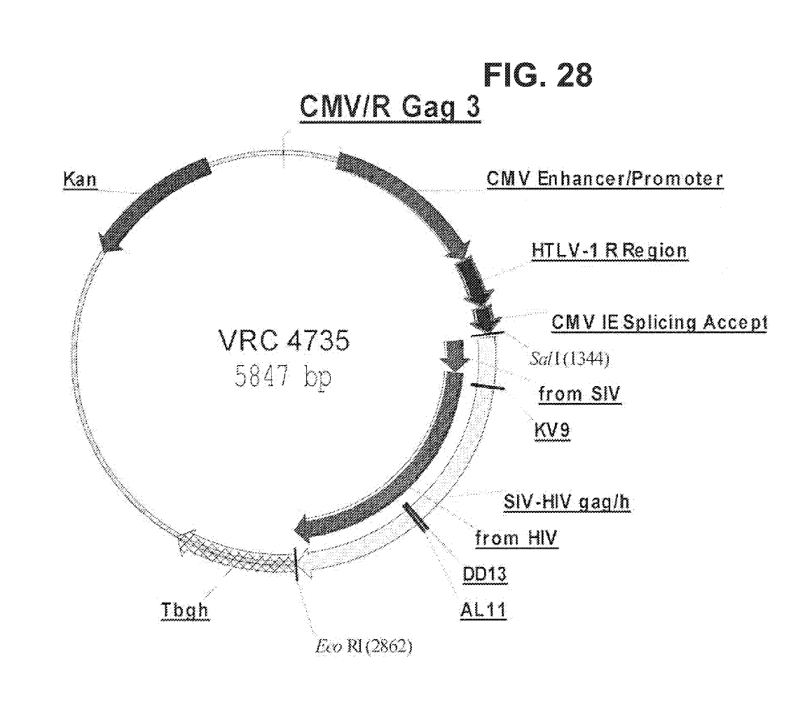

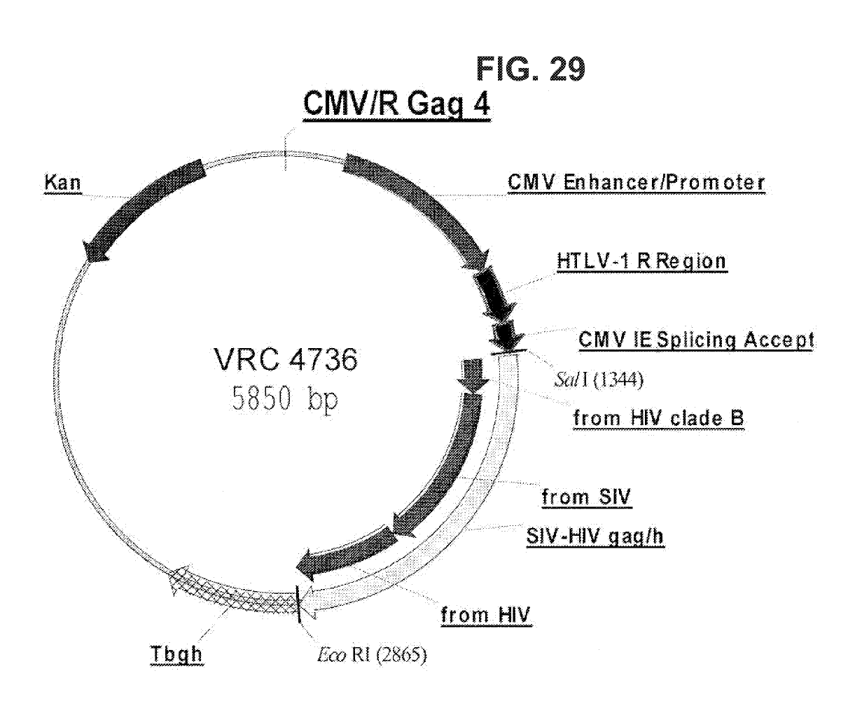

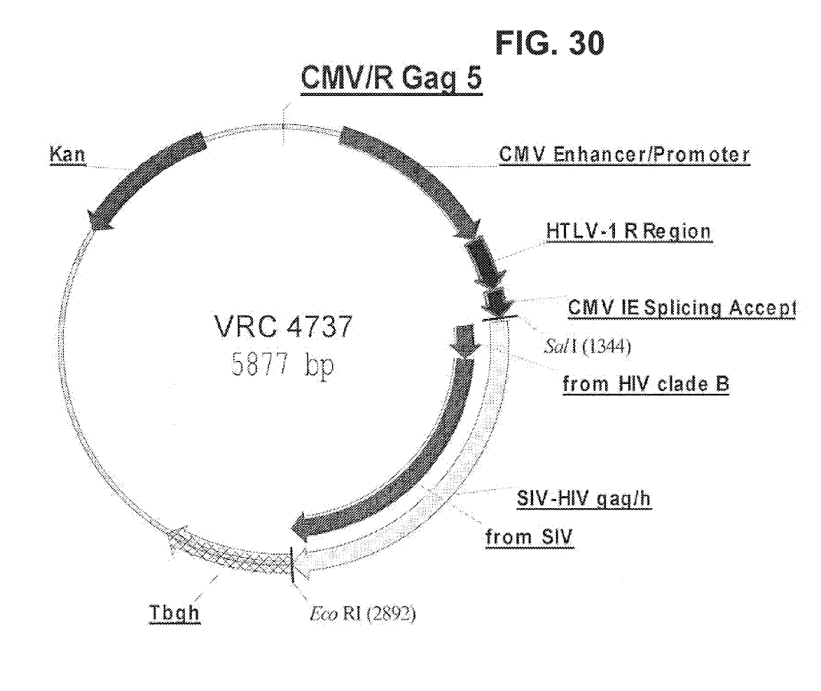

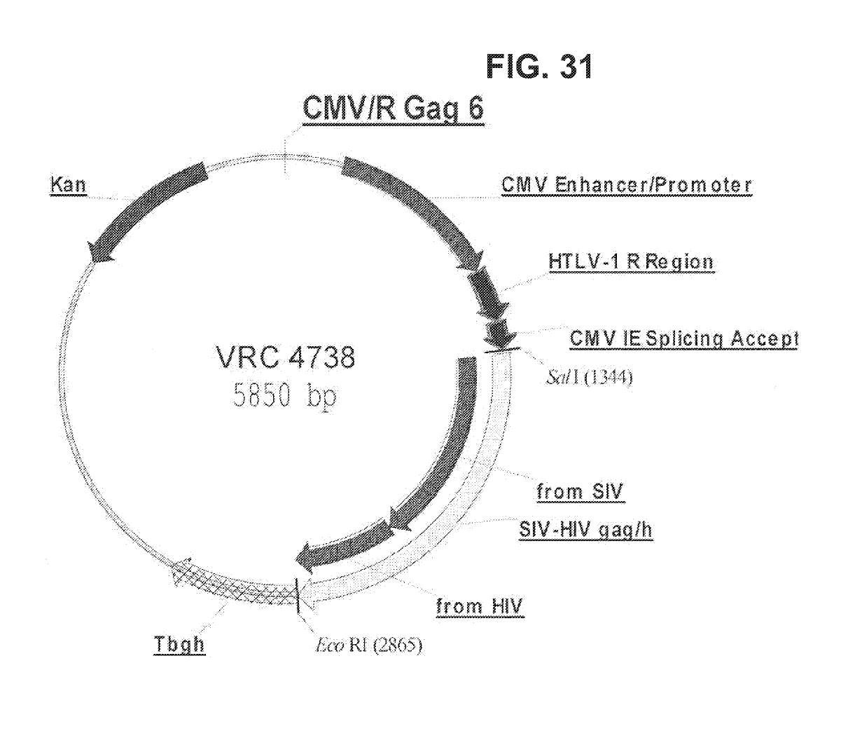

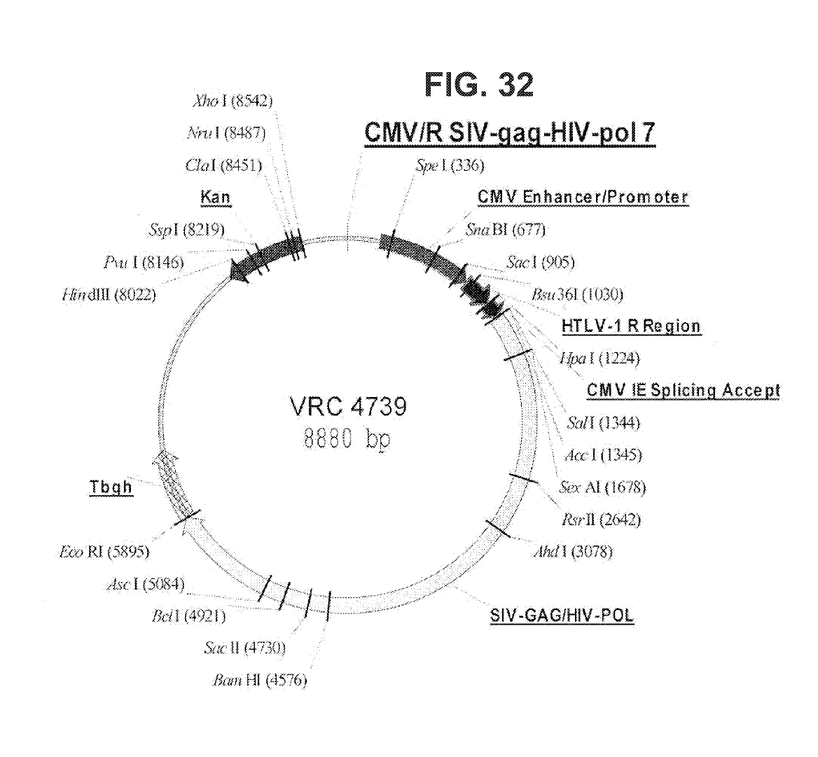

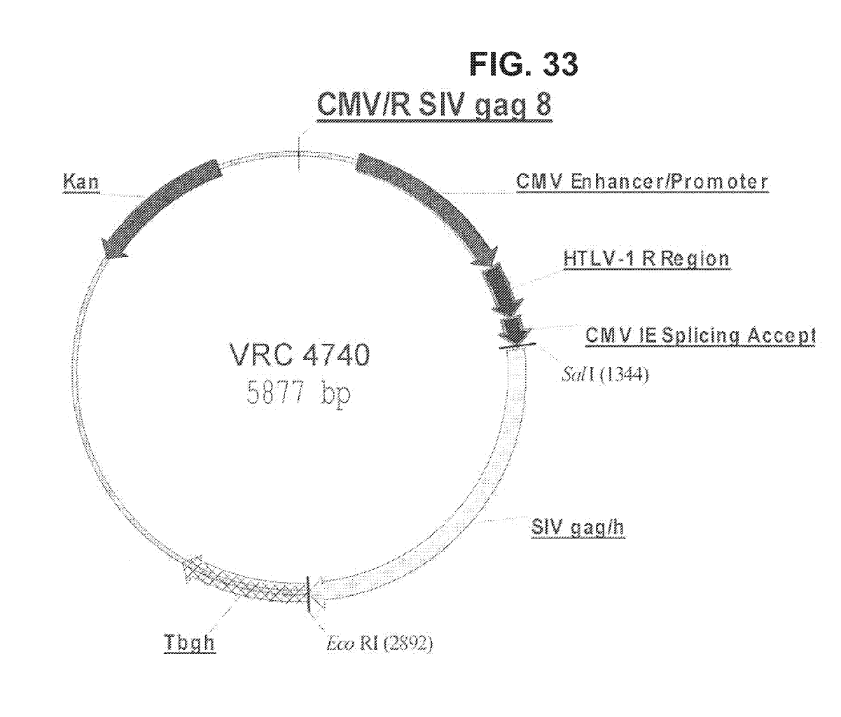

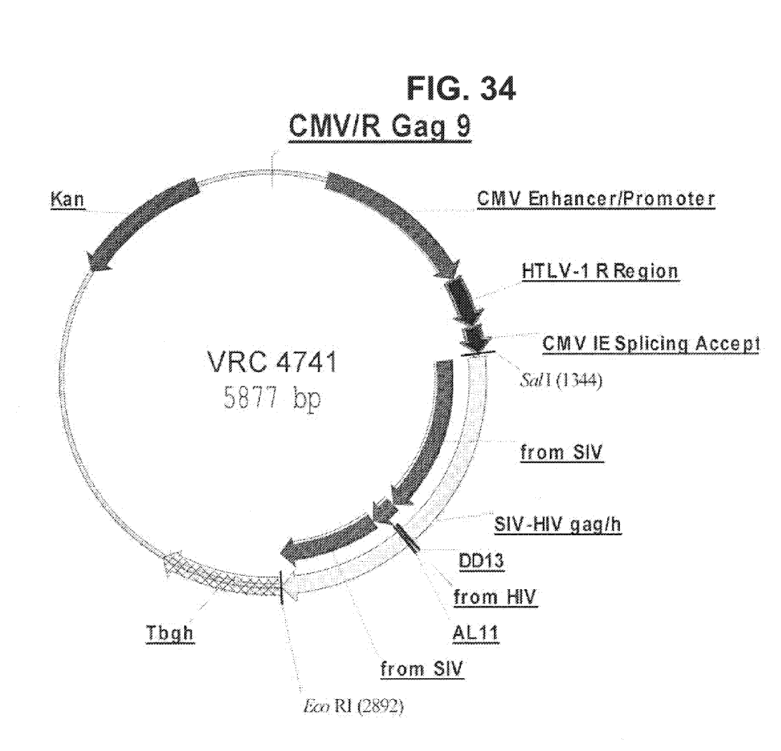

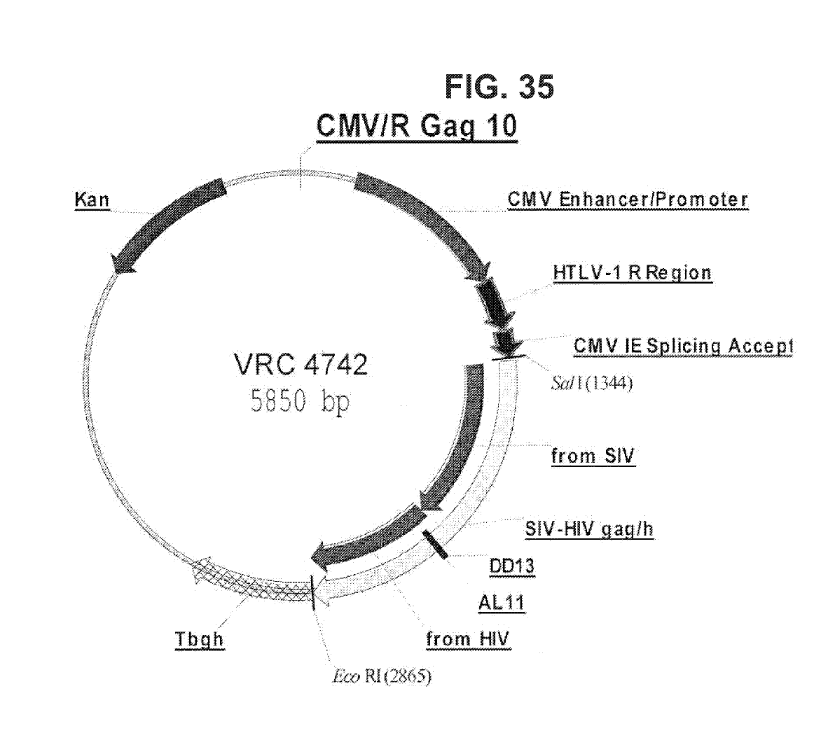

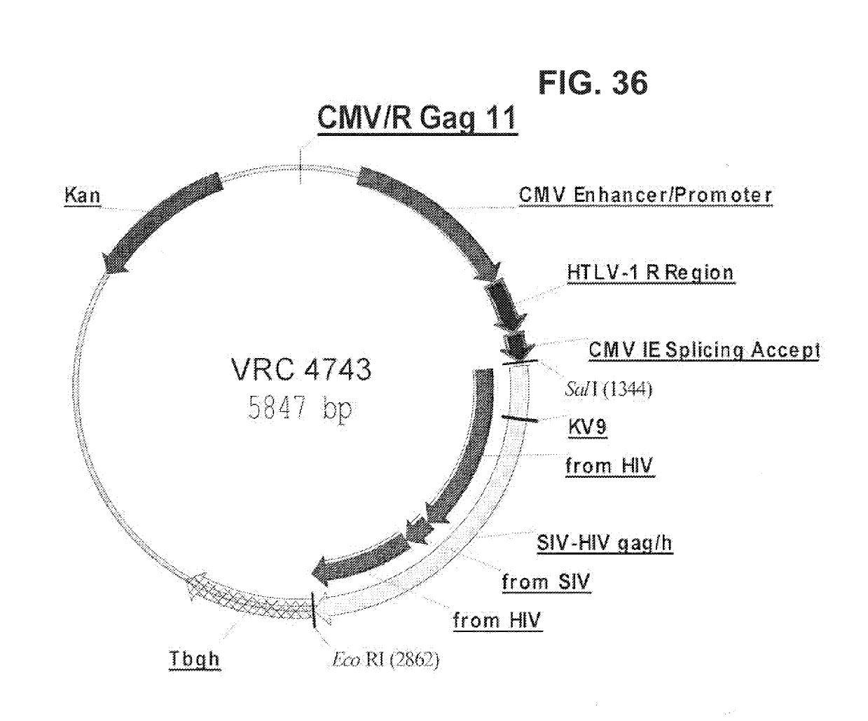

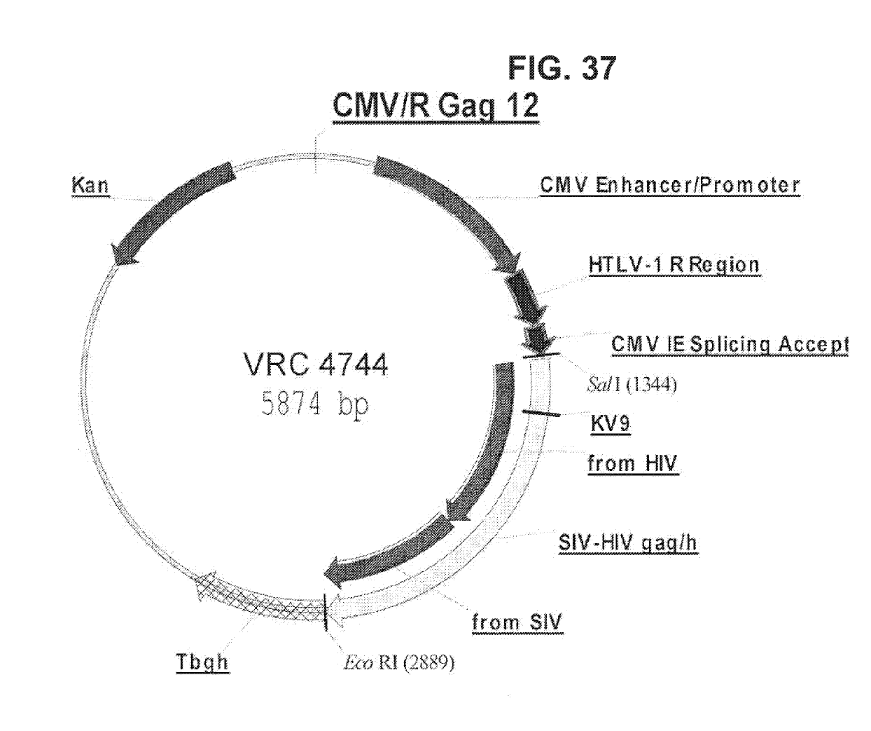

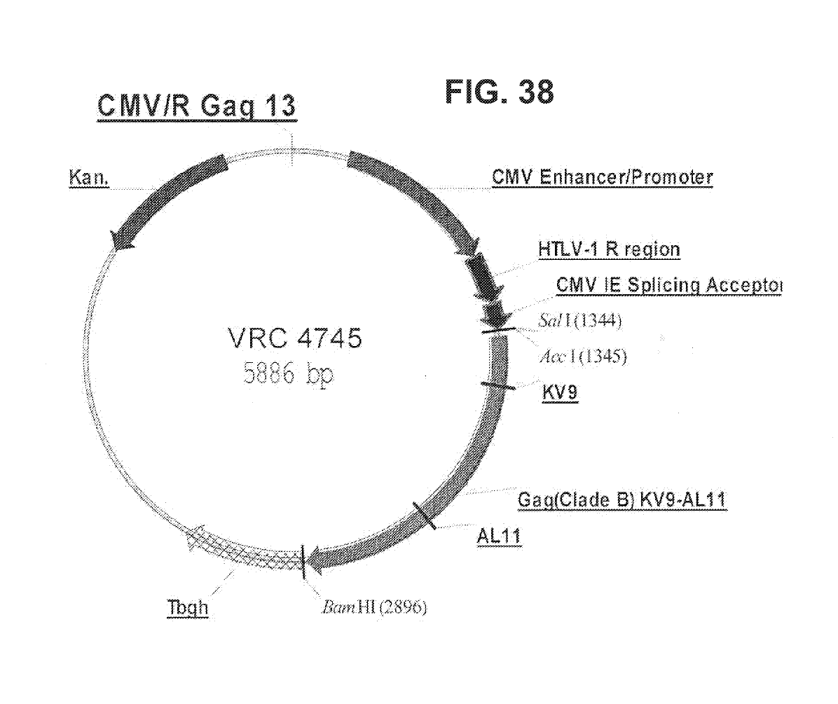

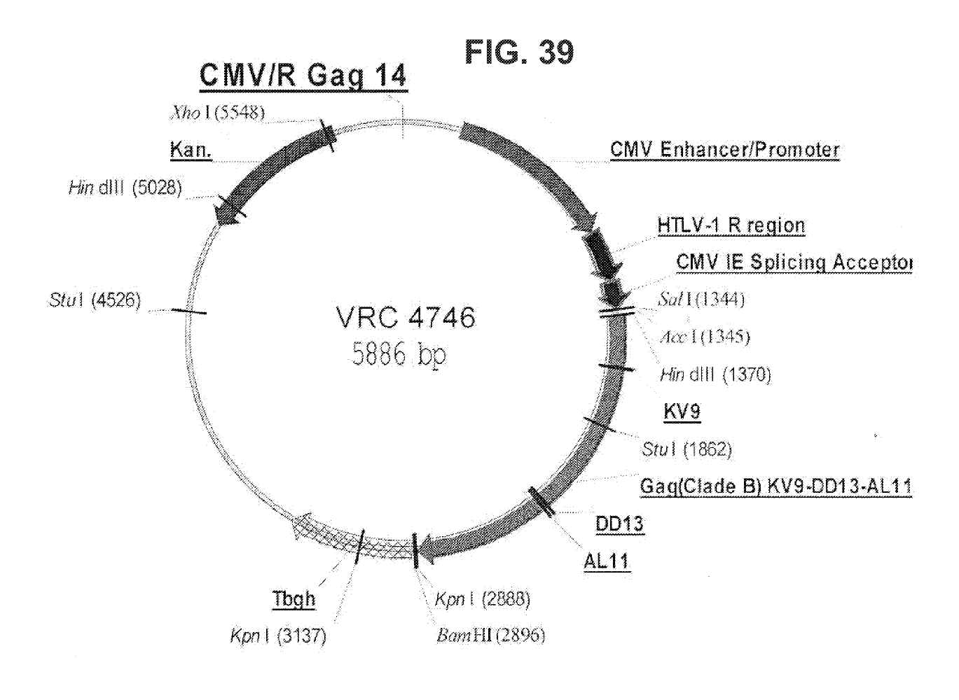

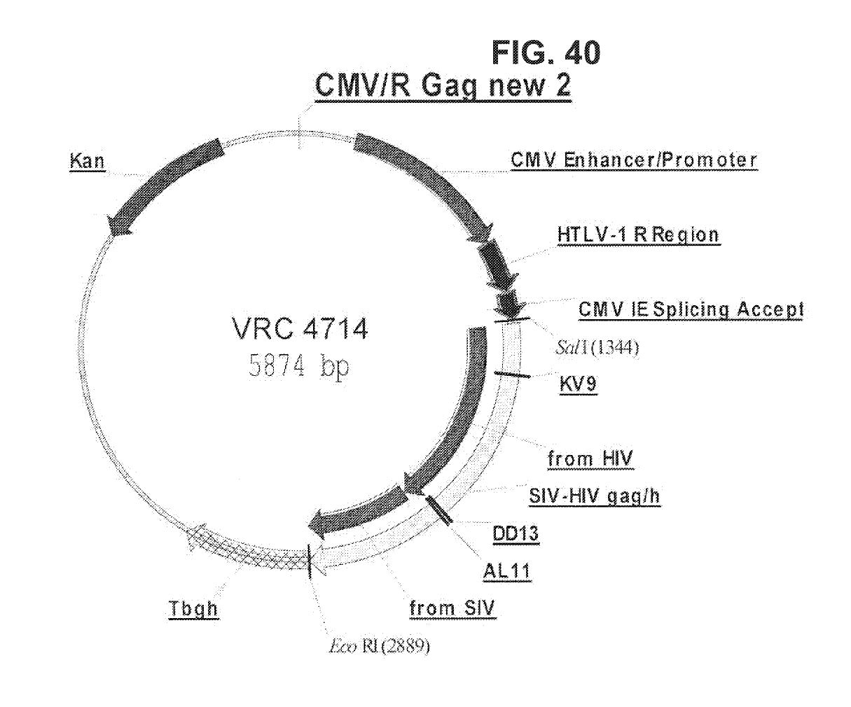

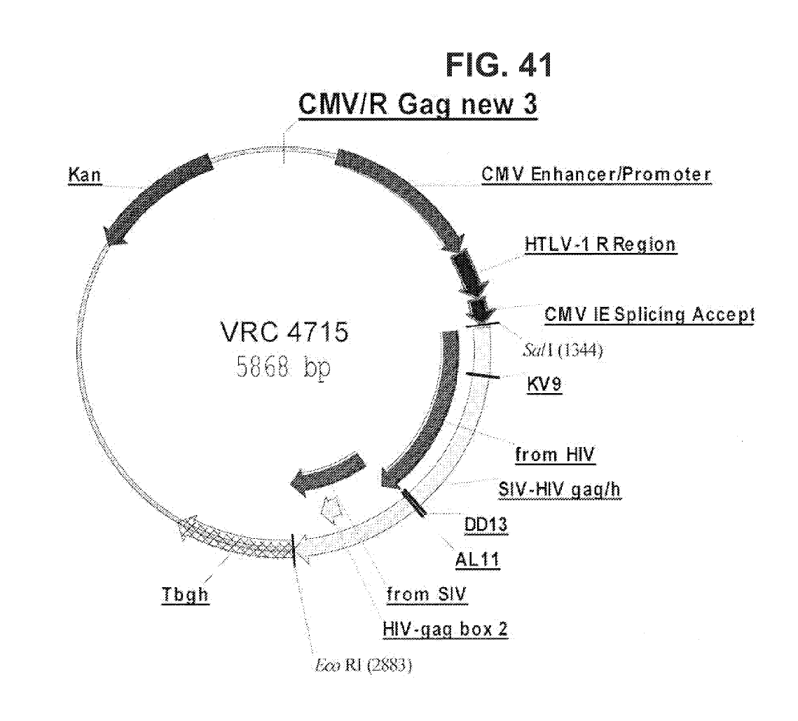

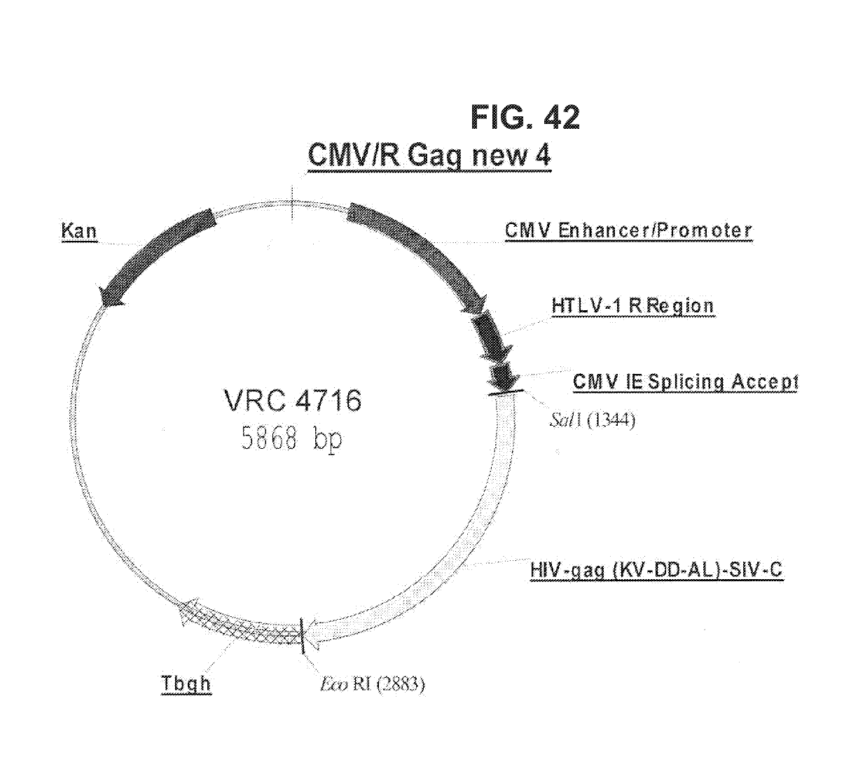

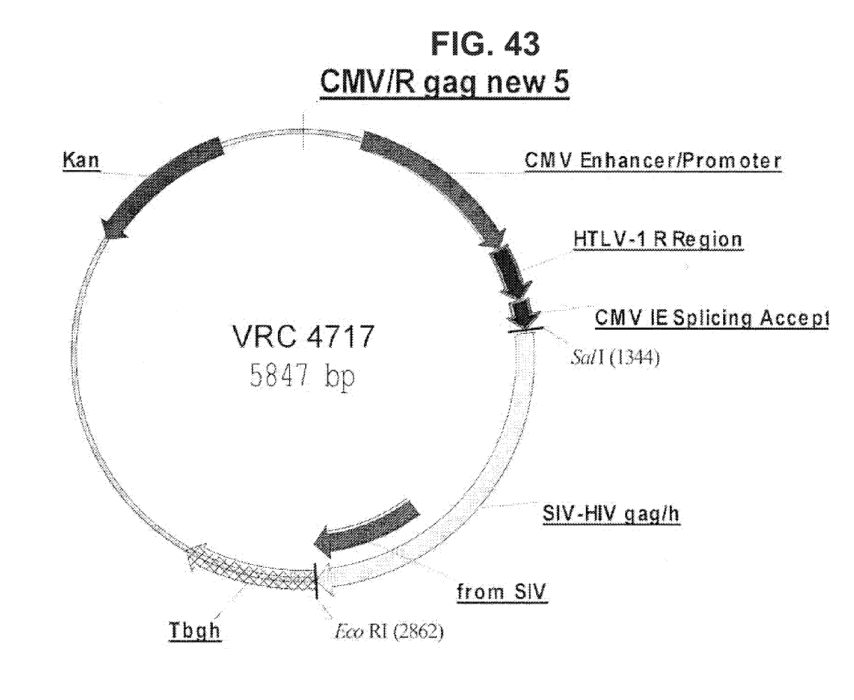

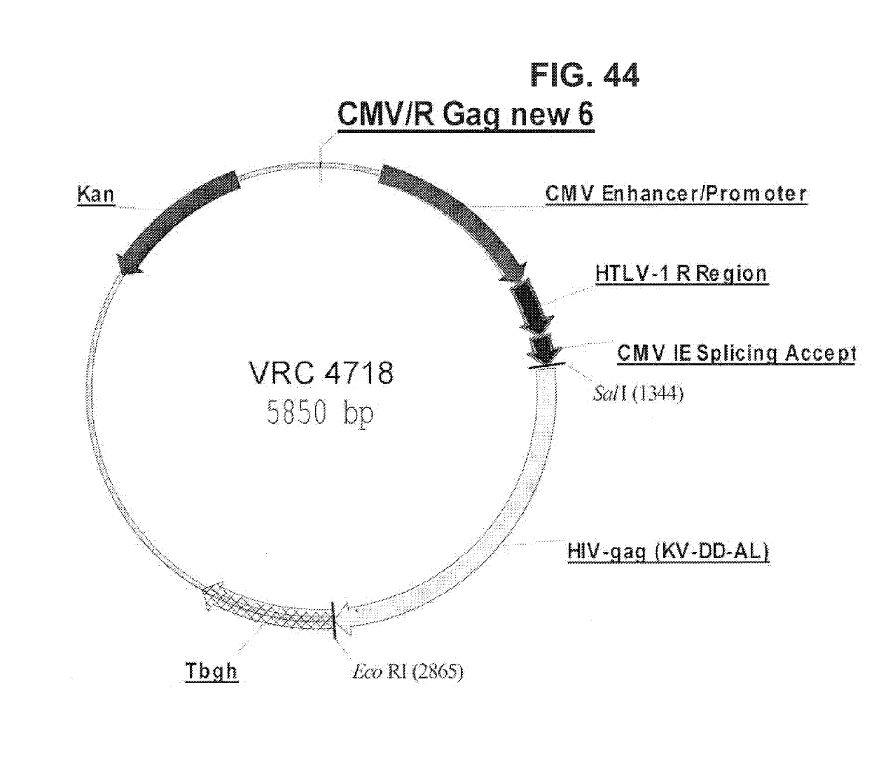

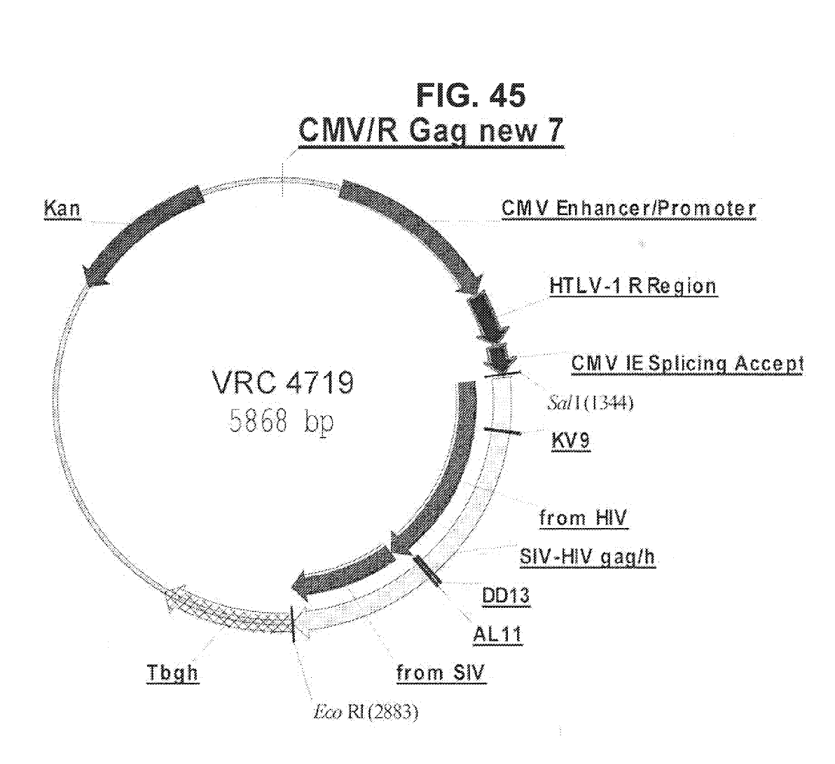

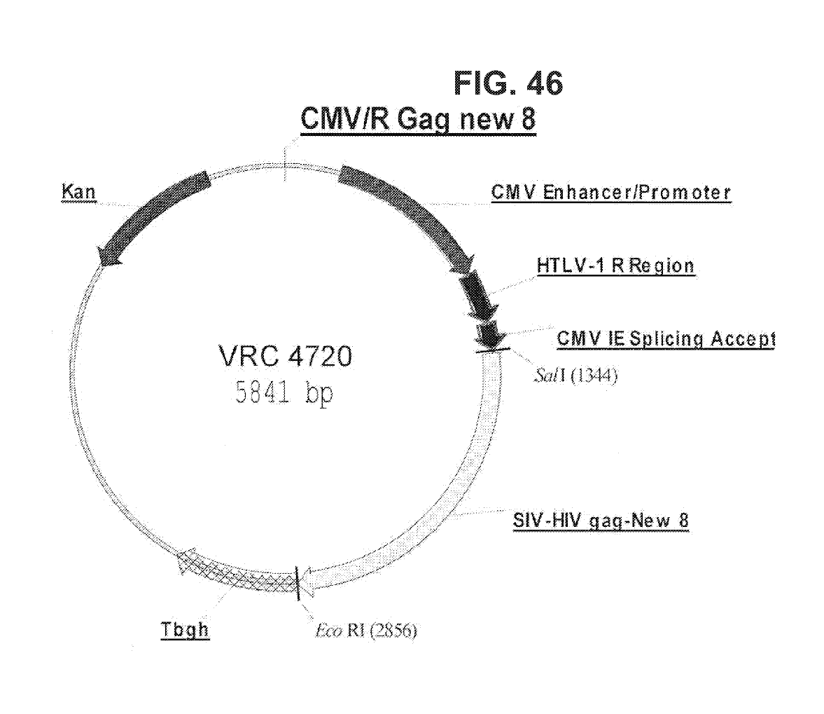

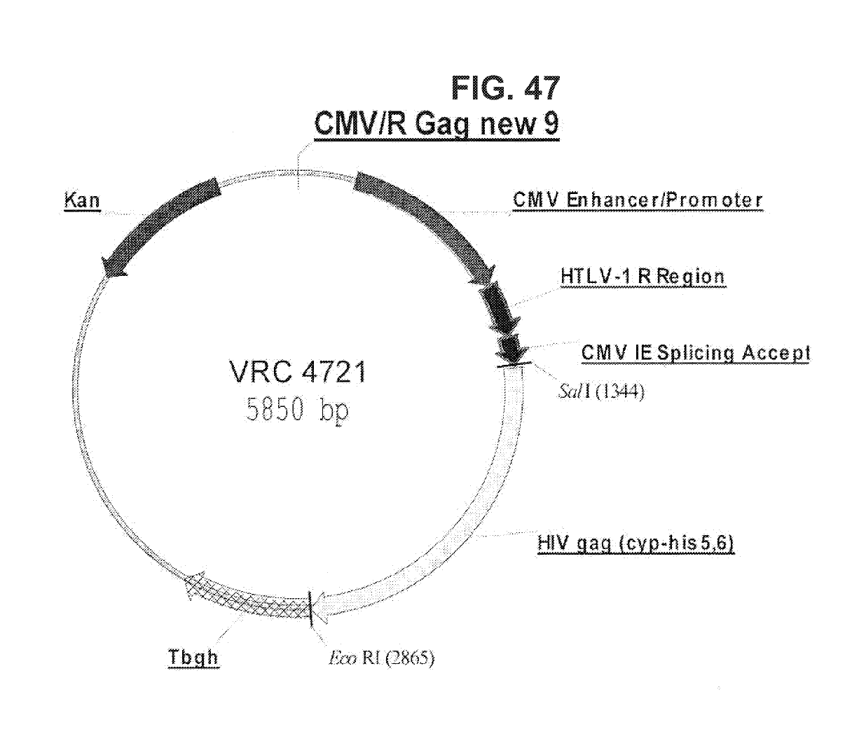

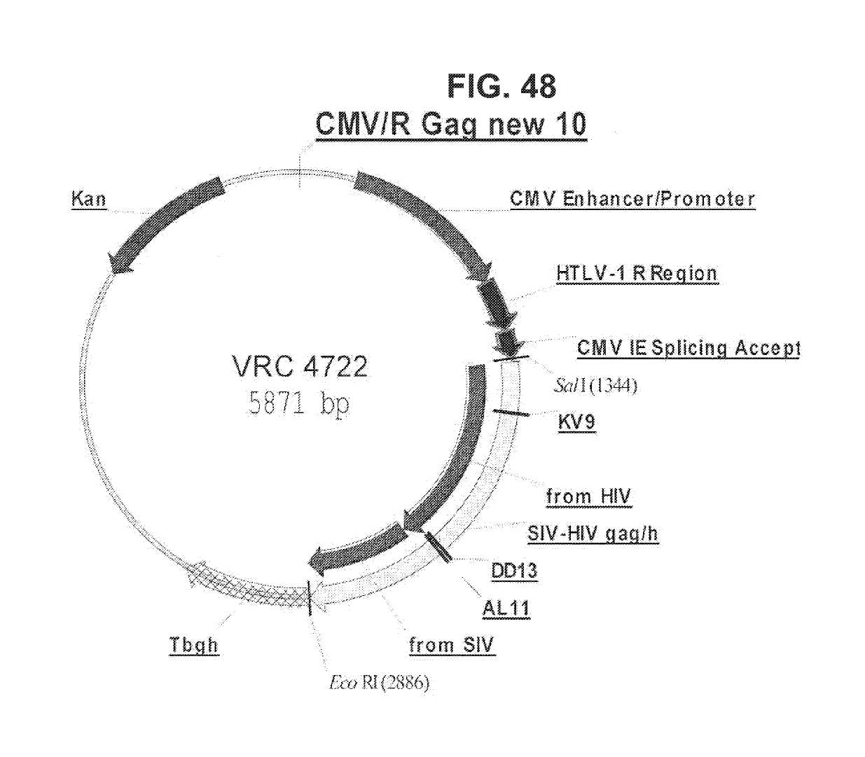

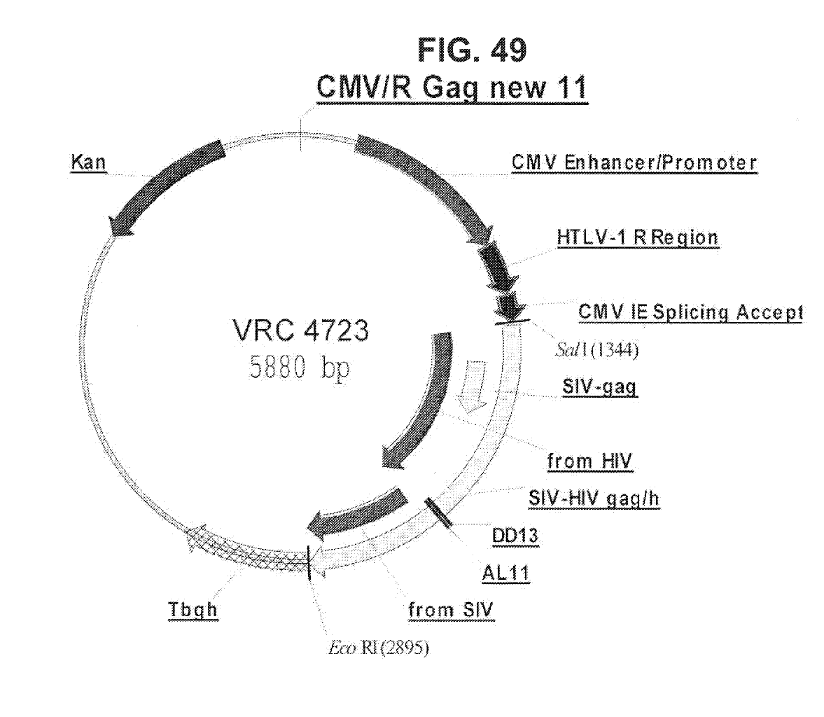

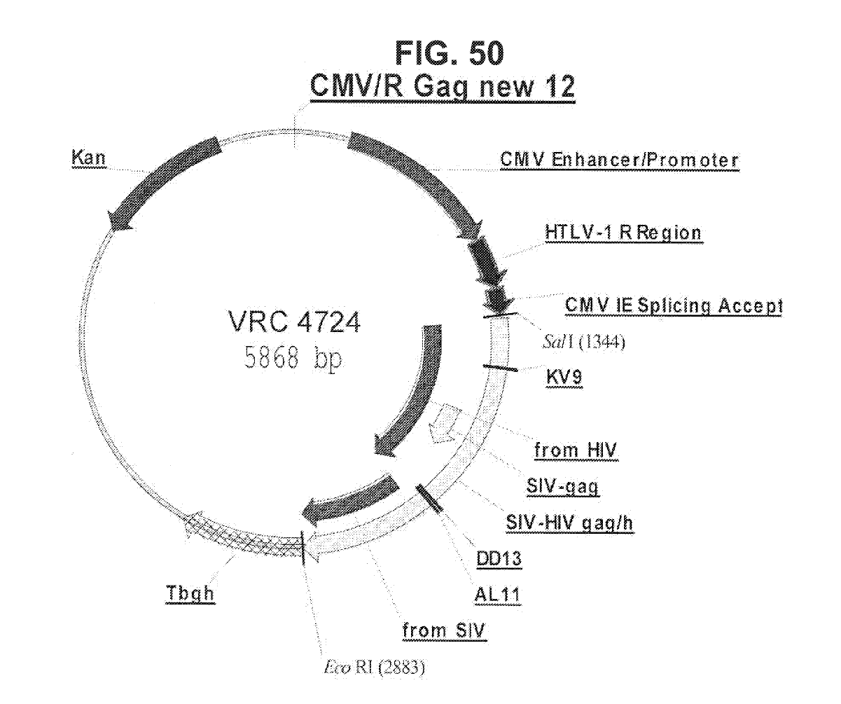

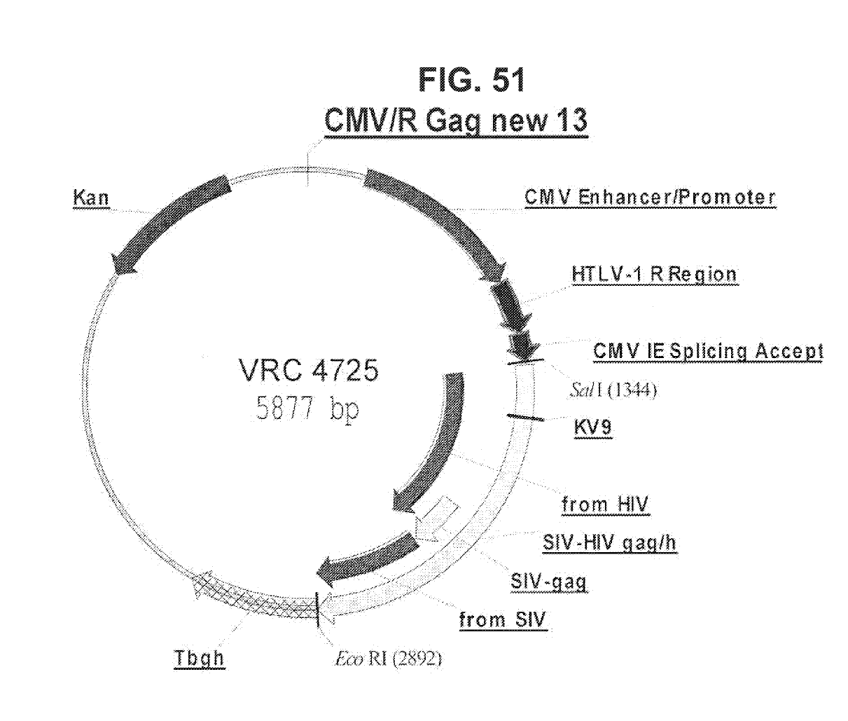

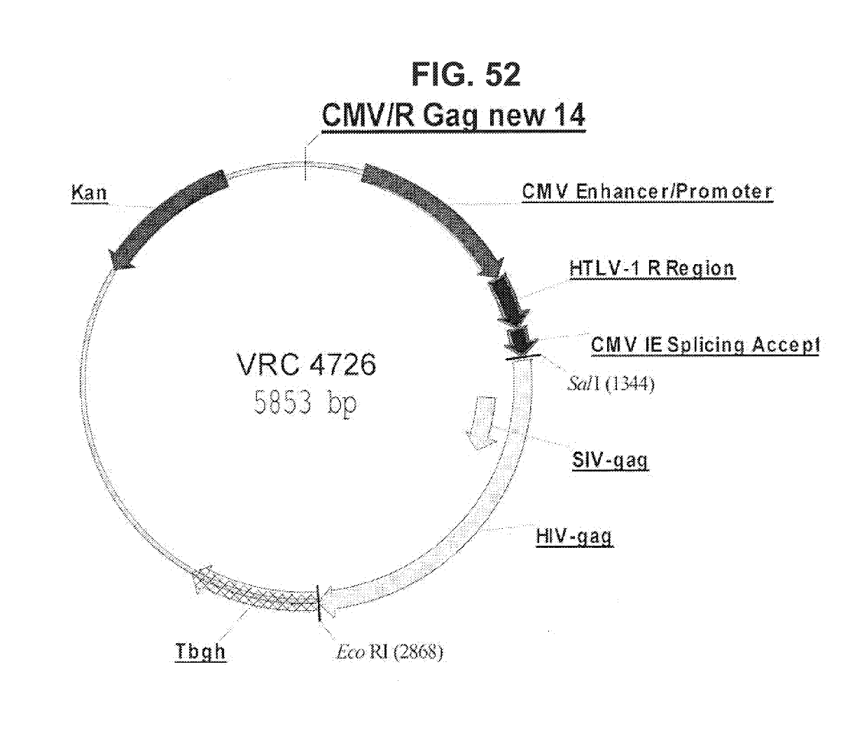

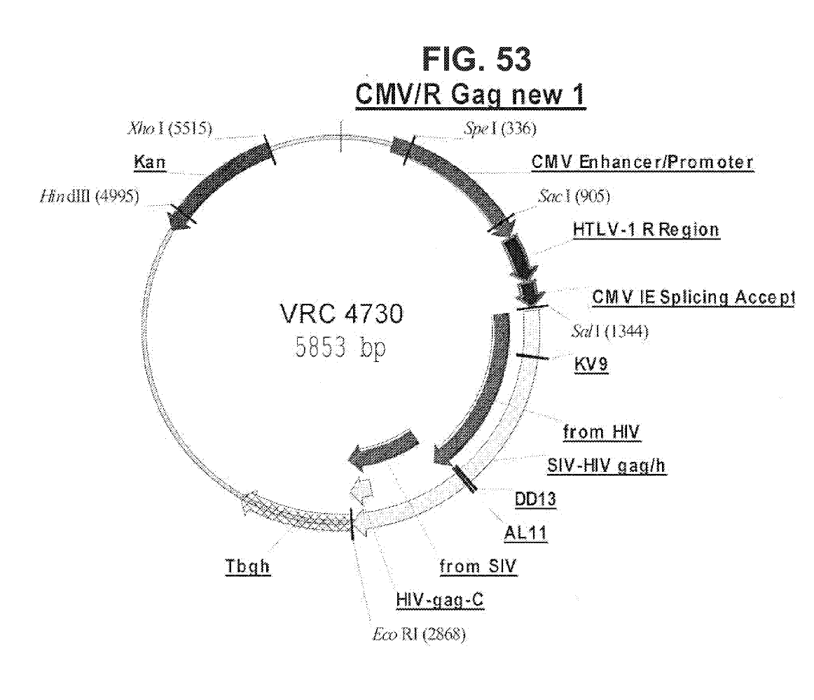

The wild-type mosaic Gag and the N5-modified mosaic Gag adenoviral vector constructs were further compared by ICS after only a single immunization. The CD4 responses elicited by the mosaic Gag and N5-modified mosaic Gag adenoviral vector constructs were limited (FIG. 5A). The peptide 57-298 was the common CD4 epitope, and only N5-modified mosaic Gag adenoviral vector construct elicited one additional CD4 epitope with a significant high response, which exhibited a very weak response in the wild-type mosaic Gag adenoviral vector construct, at aa 259 in Gag PTE #7 (FIG. 5A and FIG. 6A). However, this 7-259 peptide was also a common subdominant epitope in the plasmid construct prime/adenoviral vector construct boost immunization regimen of wild-type Gag and the mosaic Gag N5 described above (FIG. 3A). These results suggest that a single immunization with an adenoviral vector construct may not be strong enough to elicit this common CD4 epitope. In contrast, the CD8 responses elicited one common dominant epitope by the mosaic Gag and N5-modified mosaic Gag adenoviral vector constructs at aa 194 of Gag PTE #51, and the same position epitope was found in the plasmid construct prime/adenoviral vector construct boost immunization regimen (FIG. 3B). However, the N5-modified mosaic Gag elicited two additional epitopes not found in the wild-type, at amino acid residue (aa) 348 of Gag PTE#45 and at aa 354 of Gag PTE #76 (FIG. 5B, FIG. 6B). TNF-α response analysis confirmed the results of the IFN-γ responses (FIGS. 12A and 12B).

The results of this example demonstrates that the N5 modification of HIV mosaic Gag proteins elicits T cell responses to additional epitopes as compared to the wild-type mosaic Gag in B6D2F1 mice after different vaccination regimens.

### Example 4

This example describes the immunogenicity of a mosaic Gag protein in combination with a mosaic Env protein delivered to mice via recombinant adenoviral vector constructs.

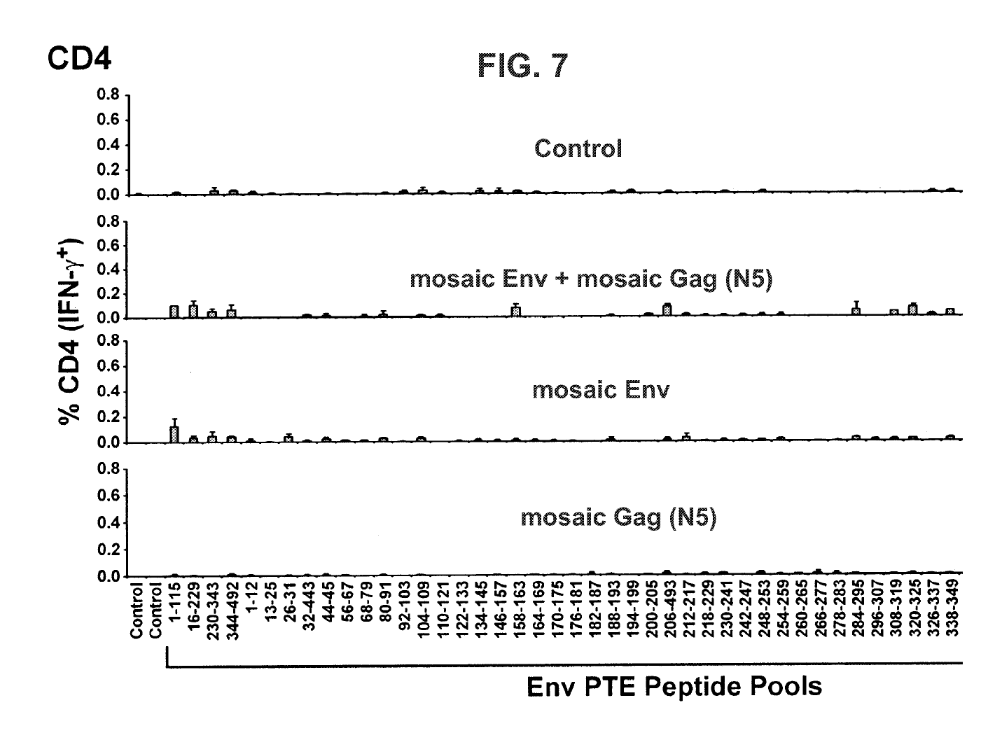

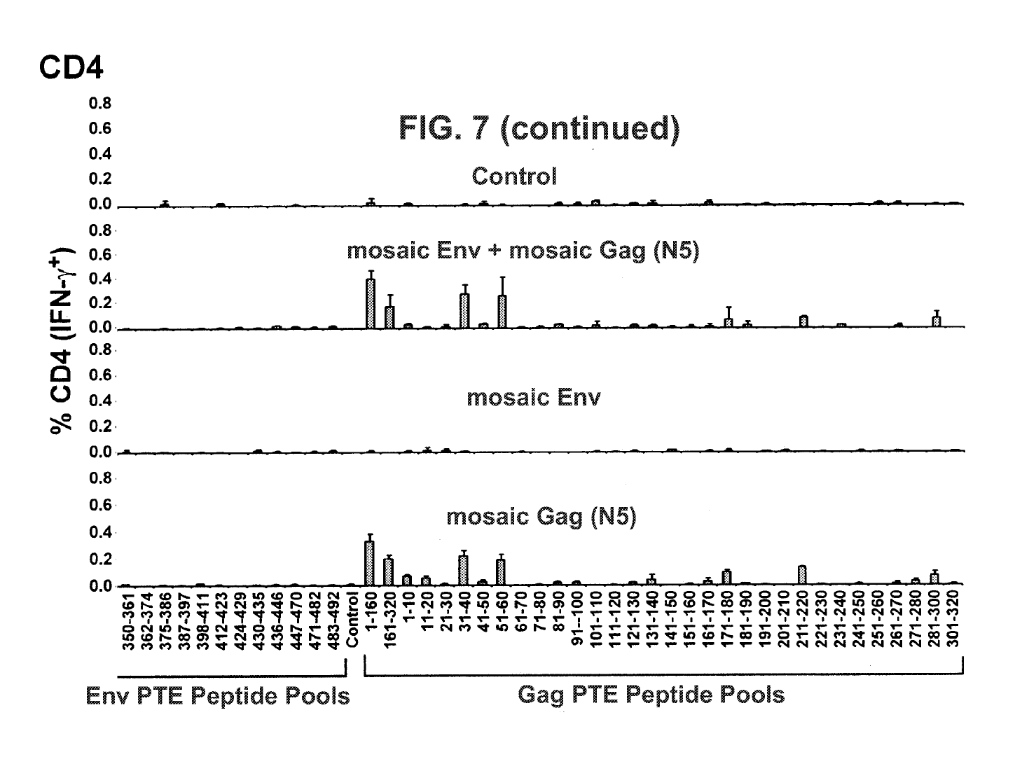

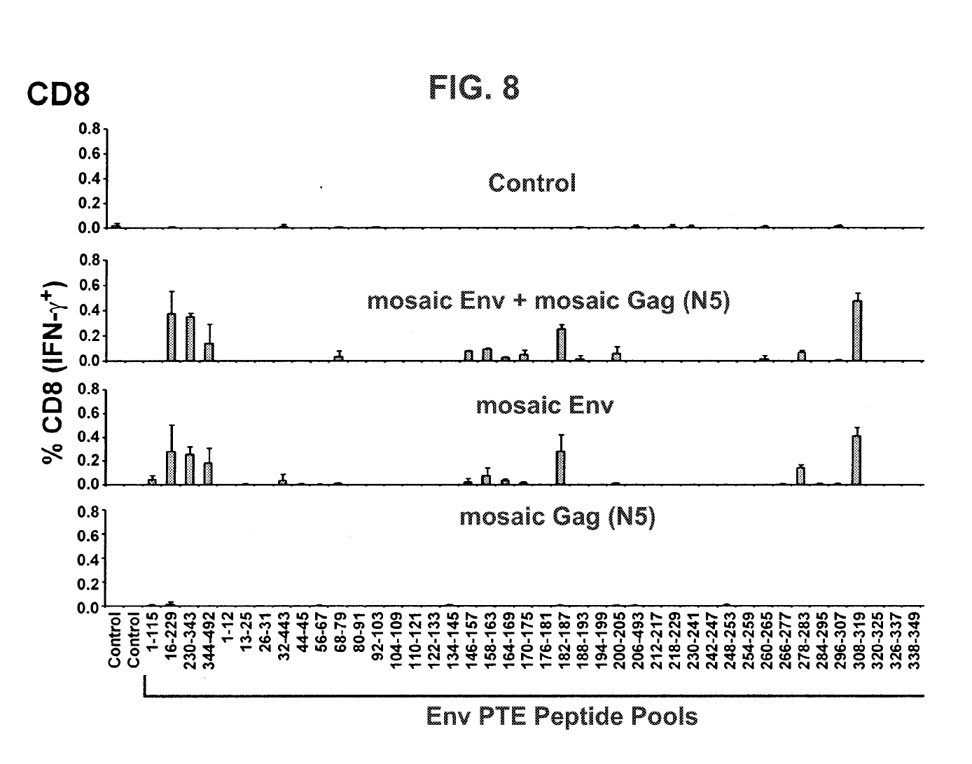

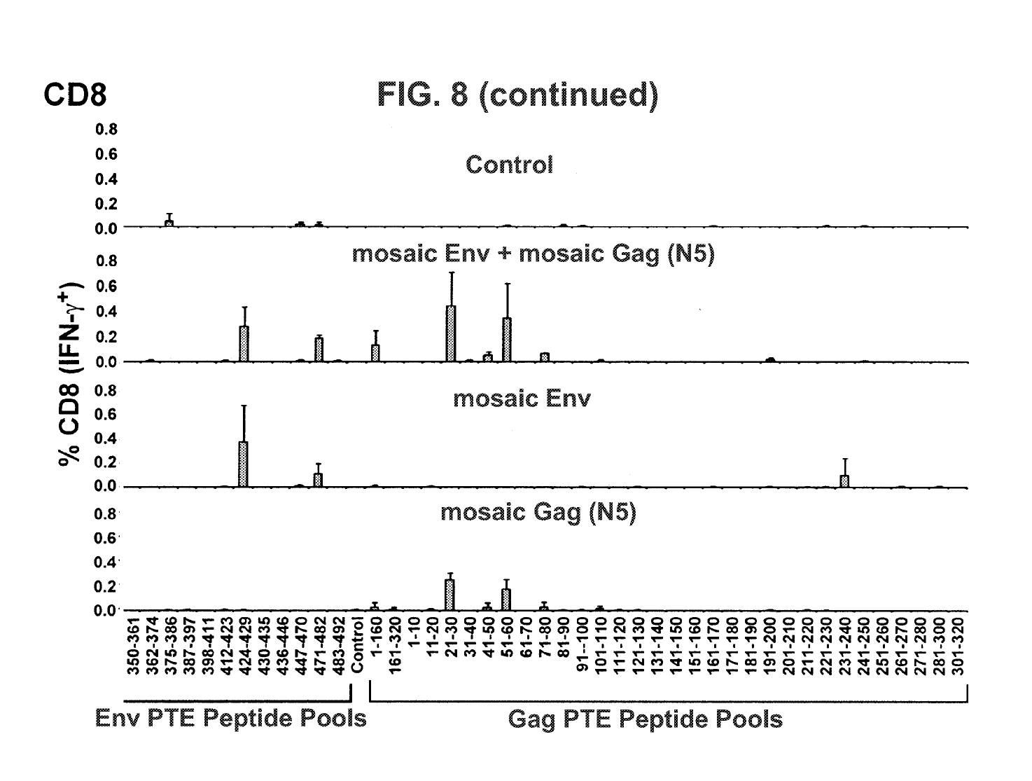

B6D2F1 mice were injected one time with a recombinant adenoviral vector construct encoding (i) a mosaic Gag N5 polypeptide (i.e., SEQ ID NO: 18 or SEQ ID NO: 19), or (ii) a mosaic Env polypeptide (SEQ ID NO: 98 or SEQ ID NO: 100), separately and in various combinations. T cell responses were determined three weeks after immunization. In particular, 320 Gag PTE peptides were grouped into 32 pools of 10 PTE peptides and the 492 Env PTE peptides were grouped into 82 pools of 6 peptides, and all pools were analyzed by ICS as previously described. The combination of the two mosaic Gag N5 polypeptides and the two mosaic Env polypeptides elicited CD4 and CD8 responses similar to administration of the adenoviral vector constructs encoding the two mosaic Env polypeptides alone or the adenoviral vector constructs encoding the two mosaic Gag N5 polypeptides alone (FIG. 7 and FIG. 8).

The results of this example demonstrate that expression of two mosaic Env polypeptides in combination with two mosaic Gag N5 polypeptides in mice does not affect the magnitude and breadth of the induced T cell response, as compared to expression of either antigens alone.

### Example 5

This example demonstrates that the N5-modification enhances expression of a nucleic acid sequence encoding a Gag polypeptide.

293 cells transfected with nucleic acid sequences encoding an N5-modified non-mosaic Gag polypeptide and an N5-modified mosaic Gag polypeptide (described above) were studied by electron microscopy to determine any differences in appearance between their Gag-forming particles. There was no significant difference in the appearance of Gag-forming particles from N5-modified Gag compared to that of N5-modified non-mosaic Gag.

To further understand the mechanism by which the N5-modification enhances the T cell response, potential expression differences between a wild-type Gag polypeptide (mosaic and non-mosaic) and an N5-modified Gag polypeptide (mosaic and non-mosaic) were analyzed in various cells, including human CD4+ T cells and mouse myoblast cell line C2C12. Human CD4 T cells were isolated as previously described (Cheng et al., PLoS Pathog., 3(2): e25 (2007)). Human buffy coat cells were obtained from the National Institutes of Health Clinical Center Blood Bank. Human CD4 T cells were isolated by magnetic cell sorting with CD4+ T Cell Isolation Kit II (Miltenyi Biotec, Gladbach, Germany). Mice myoblast cell line C2C12 was obtained from ATCC (Manassas, Va.) and cultured as recommended. Plasmid constructs containing the following sequences were generated: (i) SIV wild-type Gag, (ii) wild-type clade B HIV Gag (SEQ ID NO: 13), (iii) wild-type mosaic Gag (SEQ ID NO: 16), and (iv) N5-modified mosaic Gag (SEQ ID NO: 18). The plasmid constructs were transfected into human CD4 T cells by Amaxa Human T cell Nucleofector Kit (Lonza, Basel, Switzerland) as recommended by the manufacturer. C2C12 cells were transfected with the same plasmid constructs by Lipofectamine 2000 (Invitrogen, Carlsbad, Calif.) as recommended by the manufacturer. Gag proteins in cell lysates and supernatants were collected 48 hours post-infection and were quantified by an HIV-1 P24 ELISA kit (PerkinElmer, Waltham, Mass.).

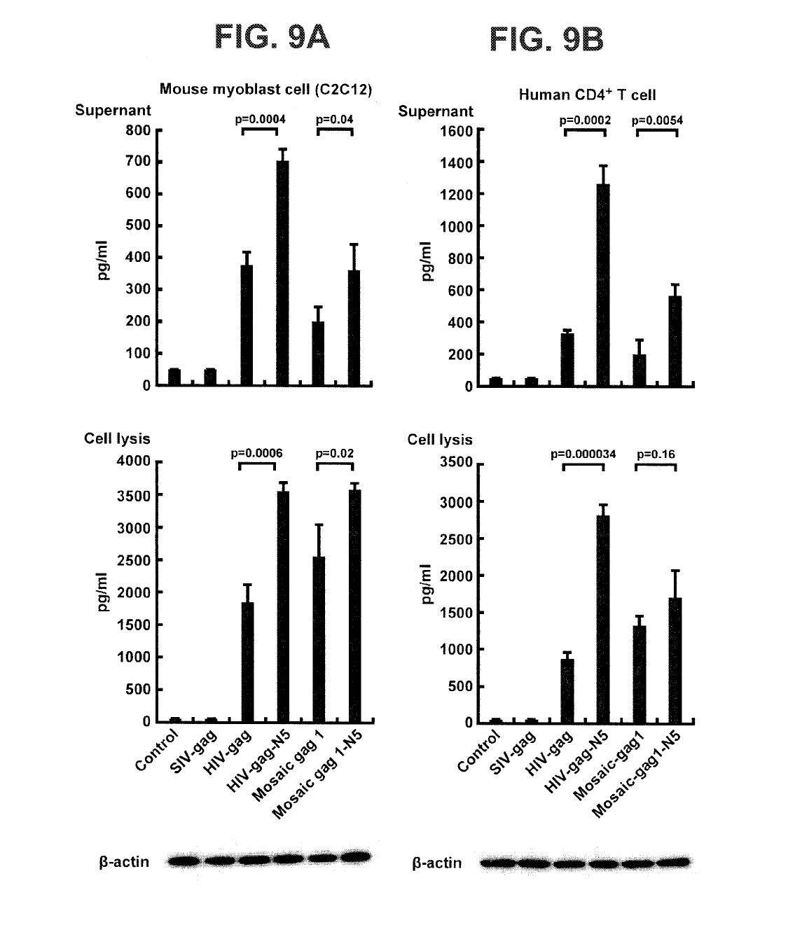

In C2C12 cells, an N5-modified non-mosaic Gag plasmid construct expressed 30% more Gag protein in cell lysates (FIG. 9A) and expressed 70% more Gag protein in supernatants (FIG. 9A), as compared to the plasmid construct enocoding a non-mosaic wild-type Gag polypeptide. In addition, the plasmid construct encoding the N5-modified mosaic Gag protein expressed 40% more Gag protein in cell lysates (FIG. 9A) and expressed 70% more Gag protein in supernatants (FIG. 9A), as compared to the plasmid construct encoding a wild-type mosaic Gag protein.

In human CD4+ T cells from one donor, the plasmid construct expressing a non-mosaic N5-modified Gag protein expressed 230% more Gag protein in cell lysates and expressed 270% more Gag protein in supernatants, as compared to the plasmid construct encoding a non-mosaic wild-type Gag protein (FIG. 9B). In addition, the plasmid construct encoding an N5-modified mosaic Gag protein expressed 20% more Gag protein in cell lysates (FIG. 9B) and expressed 200% more Gag protein in supernatants (FIG. 9B), as compared to the plasmid construct encoding a wild-type mosaic Gag protein.

The results of this example demonstrate that both non-mosaic and mosaic N5-modified Gag proteins are expressed more efficiently than wild-type Gag proteins, which may contribute to the observed differences in immunogenicity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified nucleic acid molecule comprising the nucleic acid sequence of SEQ ID NO: 1, SEQ ID NO: 2, SEQ ID NO: 3, or SEQ ID NO: 4.

2. (canceled)

3. A polypeptide encoded by a nucleic acid molecule of claim 1.

4. An isolated or purified nucleic acid molecule comprising a nucleic acid sequence that encodes the polypeptide of claim 3.

5. A construct comprising the nucleic acid molecule of claim 1, wherein the construct is suitable for expressing an HIV-1 polypeptide.

6. The construct of claim 5, wherein the construct is a plasmid vector.

7. The construct of claim 6, wherein the construct comprises SEQ ID NO: 8, SEQ ID NO: 9, SEQ ID NO: 11, or SEQ ID NO: 12.

8. The construct of claim 5, wherein the construct is a viral vector construct.

9. The construct of claim 8, wherein the viral vector construct is an adenovirus vector construct.

10. The construct of claim 9, wherein the adenovirus vector construct is selected from the group consisting of a human adenovirus vector construct, a simian adenovirus vector construct, and a chimpanzee adenovirus vector construct.

11. An isolated host cell comprising the construct of claim 5, wherein the host cell is suitable for expressing an HIV-1 polypeptide.

12. A composition capable of eliciting an immune response against HIV-1 comprising (a) the nucleic acid molecule of claim 1 and (b) a pharmaceutically acceptable carrier.

13. A syringe comprising the composition of claim 12.

14. A needleless delivery device comprising the composition of claim 12.

15. A method of inducing an immune response against HIV-1 in a mammal, which method comprises administering the nucleic acid molecule of claim 1 to a mammal, whereupon an immune response against HIV-1 is induced in the mammal.

16. A method of inducing an immune response against HIV-1 in a mammal, which method comprises administering the polypeptide of claim 3 to a mammal, whereupon an immune response against HIV-1 is induced in the mammal.

17. A method of inducing an immune response against HIV-1 in a mammal, which method comprises administering the composition of claim 12 to a mammal, whereupon an immune response against HIV-1 is induced in the mammal.

18. A composition capable of eliciting an immune response against HIV-1 comprising (a) the construct of claim 5 and (b) a pharmaceutically acceptable carrier.
